SEQUENCE LISTING (18 sequences)

SEQ ID NO: 1 - DNA, 22 bp, Artificial Sequence
  synthetic construct
aaccacacaacctactacctca

SEQ ID NO: 2 - DNA, 22 bp, Artificial Sequence
  synthetic construct
aaccacacaacctactacctca

SEQ ID NO: 3 - DNA, 22 bp, Artificial Sequence
  synthetic construct
tgaggtagtaggttgtgtggtt

SEQ ID NO: 4 - RNA, 22 nt, Artificial Sequence
  synthetic construct
ugagguaguagguugugugguu

SEQ ID NO: 5 - DNA, 22 bp, Artificial Sequence
  synthetic construct
tagcttatcagactgatgttga

SEQ ID NO: 6 - RNA, 22 nt, Artificial Sequence
  synthetic construct
uagcuuaucagacugauguuga

SEQ ID NO: 7 - DNA, 40 bp, Artificial Sequence
  synthetic construct
  modified_base (7)..(7): I
  modified_base (31)..(31): I
  modified_base (35)..(35): I
  modified_base (39)..(39): I
tgaaagagttttaataatagaattttaaaaaactagtaaa

SEQ ID NO: 8 - DNA, 47 bp, Artificial Sequence
  synthetic sequence
ccttatttctaccagtcttttaaaattctattattaaaaccctttca

SEQ ID NO: 9 - DNA, 19 bp, Artificial Sequence
  synthetic construct
tgaggtagtaggttgtgtg

SEQ ID NO: 10 - DNA, 18 bp, Artificial Sequence
  synthetic construct
tgactaaaatgtccccgg

SEQ ID NO: 11 - DNA, 22 bp, Artificial Sequence
  synthetic construct
cgttccataatgttgtaaccag

SEQ ID NO: 12 - DNA, 39 bp, Artificial Sequence
  synthetic construct
gaagctggctaccgagactcccaaaagcaacatgaccga

SEQ ID NO: 13 - DNA, 38 bp, Artificial Sequence
  synthetic construct
gcgatctctgaacggcgattcctgcaactgtgacgaag

SEQ ID NO: 14 - DNA, 24 bp, Artificial Sequence
  synthetic construct
gccgcataatttaatgccttgtca

SEQ ID NO: 15 - DNA, 19 bp, Artificial Sequence
  synthetic construct
acgcgaaagataccgctct

SEQ ID NO: 16 - DNA, 39 bp, Artificial Sequence; identical to SEQ ID NO: 12
  synthetic construct

SEQ ID NO: 17 - DNA, 60 bp, Artificial Sequence
  synthetic construct
cgccaagctttttaacagtggccttattaaatgacttctctaaaggatcccgggtaccga

SEQ ID NO: 18 - DNA, 20 bp, Artificial Sequence
  synthetic construct
gaccccaaaatcagcgaaat

(19) United States
(12) Patent Application Publication
(10) Pub. No.: US 2023/0080531 A1
(43) Pub. Date: Mar. 16, 2023

(54) ULTRASENSITIVE BIOSENSOR USING BENT AND CURVED FIELD EFFECT TRANSISTOR BY DEBYE LENGTH MODULATION

(71) Applicant: The Board of Trustees of the University of lllinois, Urbana, IL (US)

(72) Inventors: Michael HWANG, Urbana, IL (US); Rashid BASHIR, Urbana, IL (US); Mohammad HEIRANIAN, Urbana, IL (US); Sungwoo NAM, Urbana, IL (US); Narayan ALURU, Urbana, IL (US); Arend VAN DER ZANDE, Urbana, IL (US); Catherine J. MURPHY, Urbana, IL (US); Jonghyun CHOI, Urbana, IL (US); Yerim KIM, Urbana, IL (US)

(21) Appl. No.: 17/800,461

(22) PCT Filed: Feb. 26, 2021
(86) PCT No.: PCT/US2021/020006

§ 371 Date: Aug. 17, 2022

Related U.S. Application Data
(60) Provisional application No. 62/982,801, filed on Feb. 28, 2020.
(60) Provisional application No. 63/029,136, filed on May 22, 2020.
(60) Provisional application No. 63/054,039, filed on Jul. 20, 2020.

(51) Int. Cl. H01L 29/772; G01N 27/414; C12Q 1/6816; C12Q 1/6876

(52) U.S. Cl. CPC H01L 29/772; G01N 27/4146; G01N 27/4145; C12Q 1/6816; C12Q 1/6876

(57) ABSTRACT

Provided are biosensors, systems and related methods of using the biosensors and systems. The biosensor comprises a field-effect transistor (FET) having a crumpled geometry to effectively increase the detection sensitivity of a target molecule in an ionic solution. A FET having a crumpled semiconductor material channel can form a π-π interaction with single stranded DNA (ssDNA) for amplification detection applications. Increasing amount of ssDNA in an amplification reaction solution is incorporated into an amplified double stranded DNA, with increasing amplification, resulting in a lower amount of ssDNA primers. The FET is contacted with the amplified solution to electrically detect an amount of ssDNA primer in the amplified solution, thereby detecting amplification based on a decreased amount of ssDNA bound to the FET. Also provided are biosensors that can detect biomolecules more generally, such as protein, polypeptides, polynucleotides, or small molecules.

### CROSS-REFERENCE TO RELATED APPLICATIONS

This application claims the benefit of and priority to U.S. provisional patent application Nos. 62/982,801, filed Feb. 28, 2020; 63/029,136 filed May 22, 2020; and 63/054,039 filed Jul. 20, 2020, each of which is incorporated by reference herein in its entirety, except to the extent inconsistent herewith.

### STATEMENT REGARDING FEDERALLY SPONSORED RESEARCH OR DEVELOPMENT

This invention was made with government support under Award Number DMR-1720633 awarded by the National Science Foundation. The government has certain rights in the invention.

### REFERENCE TO A SEQUENCE LISTING

A sequence listing containing SEQ ID NOs: 1-18 is provided herewith in a computer-readable .txt file and is specifically incorporated by reference.

### BACKGROUND OF INVENTION

Provided herein are sensors and related methods for electrical detection of material in an ionic solution using a field-effect transistor (FET) having a bent and curved (“crumpled”) channel layer positioned between source and drain electrodes. The invention is particularly useful for increasing electric detection of biomolecules and can be used in a variety of applications, including as a research and/or diagnostic tool. One specific use is for detecting molecules such as biomolecules such as proteins, small molecules and nucleic acid, including from an unprocessed biological sample.

Many techniques are available for detecting biological materials such as nucleic acids, including optical, electrical and mechanical. An issue with many of those techniques, however, relates to sensitivity, with a need for substantial pre-processing to increase the concentration of the material in the solution. Electrically-based techniques are particularly promising in that they can achieve high sensitivity. See, e.g., U.S. Pat. No. 9,376,713 (“Label free detection of nucleic acid amplification”), U.S. Pat. No. 10,527,579 (“Label free analyte detection by electronic desalting and field effect transistors”), and U.S. Pat. No. 10,175,195 (“Nanopore Sensors for Biomolecular Characterization”); U.S. Pub. Nos. 2017/0022546 (“Detection and Qualification of Methylation in DNA”), 2019/0011349 (“Label-Free Characterization Of Particles Suspended In A Fluid”).

A fundamental issue associated with electronically detecting molecules in a charged solution relates to Debye screening from counter ions in solution, also referred to as “Debye shielding” or “Debye length”. The charge of molecules in a solution can be masked by the charge of ions in solution, so that molecules can only be reliably electrically detected if they are close to the sensor surface. Outside the Debye length, charges are electrically screened. Accordingly, an increase in Debye length can provide a reduced screening effect and a corresponding more sensitive electrical detection of charged target materials, including biomolecules. The invention provided herein addresses this by effectively increasing the

Debye length for a FET used in a sensor to electrically sense a target molecule in a solution, thereby increasing sensor sensitivity.

There is also a need in the art to sensitively and reliably detect DNA amplification products. Enzymatic DNA amplification-based approaches involving intercalating DNA-binding fluorescent dyes and expensive optical detectors are the gold standard for nucleic acid detection. As components of a simplified and miniaturized system, conventional silicon-based ion sensitive field effect transistors (ISFETs) that measure decrease in pH due to generation of pyrophosphates during DNA amplification have been previously reported. Provided herein are methods and systems for selective adsorption of only single stranded DNA molecules on a FET channel surface, such as a graphene surface, to detect enzymatic DNA amplification by sensing the consumption of single stranded DNA primers, including in an isothermal amplification reaction.

### SUMMARY OF THE INVENTION

Provided herein are sensors and related methods for sensitively and accurately detecting target molecules in an ionic solution, specifically including biosensors for detecting a target biomolecule. The sensors and methods use a specially configured FET having a crumpled channel layer. The crumpled geometry can effectively increase the Debye length extending from the channel surface for biomolecules in a physiological (e.g., ionic) solution. This provides a platform for detecting extremely small amounts of biomolecules, such as DNA, RNA, microRNA, proteins, small molecules and the like in an ionic or charge-containing solution, such as unprocessed biological fluid or biologically-relevant solutions that maintain biomolecule integrity (e.g., buffers).

The biosensor comprises a field effect transistor (FET). The FET has a source electrode and a drain electrode, wherein the source and drain electrodes are separated from each other by an electrode separation distance. The biosensor is compatible with a range of electrode separation distances. A channel layer is positioned between the source electrode and the drain electrode, wherein the channel layer has a crumpled geometry. The crumpled geometry is particularly relevant for increasing the Debye length at the channel layer surface, thereby increasing detection efficiency of the system by better accommodating charge shielding associated with detection in an ionic sample. A sample reservoir is in fluidic contact with the channel layer, wherein the sample reservoir is configured to hold a sample solution. The sample reservoir may be as simple as corresponding to the volume formed between the crumpled channel and the adjacent source and drain electrodes, particularly if microfluidics are used to precisely deliver fluid sample to the crumpled channel. For a larger volume sample, the sample reservoir may be formed from reservoir walls, such as by a polymer or non-polymer material, so long as there is not an adverse impact on the crumpled channel electronic measurement. A gate electrode is configured to electrically contact the sample solution in the sample reservoir. The crumpled geometry of the channel increases a detection limit of the biosensor to charged molecules in an ionic solution.

The channel layer may be formed of a two-dimensional layer of material selected from the group consisting of: graphene, doped silicon, silicene, ultra-thin metal, germanane, MoS₂, and dichalcogenides. Preferably, the channel layer is a layer of graphene.

A support substrate layer may support the electrodes and channel layer. For example, the channel layer and electrodes may be deposited on the support substrate layer, and the support substrate layer shrinkably deformed to generate crumples in the overlying channel layer. “Support” is used broadly to refer to a material having continuous or discontinuous contact and/or continuous or discontinuous bonding with the supported component(s).

To further increase sensitivity, the biosensor may further comprise a probe anchored to the channel by a linker molecule configured to selectively bind a target molecule. For example, the probe can be selected from the group consisting of: a polynucleotide, or a peptide nucleic acid (PNA) probe, an aptamer, a protein, an antibody, and a capture agent, wherein the probe has a sequence selected to specifically bind a target molecule. For a nucleotide sequence, the probe may have a complementary sequence to at least a portion of the target molecule target sequence. Depending on the desired specificity, the length and sequence complementary homology are accordingly selected as known in the art. In this manner, the sensors and methods provided herein are compatible with any number of target sequences.

The biosensors provided herein are so sensitive that they can be configured to reliably detect a single target molecule, such as having a limit of detection that corresponds to about 1 to 1000 total nucleic acids in a sample solution that is introduced to the biosensor, including in a sample solution that is between about 1 μL and 1 mL. The sensitivity may also be described in terms of a concentration of a target molecule, with a sensitivity as low as atto- to zepto-molar range, such as a limit of detection of a biomolecule concentration of between 0.1 aM to 100 aM (corresponding to about 100-1000 DNA molecules in a 50 μL sample volume). As described herein, this high sensitivity is achieved by the crumpled geometry of the channel layer between source and drain electrodes, including from parameters associated with the channel layer, such as the material, amplitude and periodicity.

The crumpled geometry configuration, including periodicity and amplitude of bending, may be selected to provide a bandgap for an exponential change in a source drain current from a small number of charges.

The channel may have a thickness of between 1 nm to 2 mm and a surface area of between 1 mm² to 100 mm². Again, the particular geometry is selected depending on various parameters associated with the application of interest. For example, expected low concentration of target molecule in the sample may inform a smaller separation distance between the electrodes. Higher concentrations may have a larger surface area channel. The sample solution, including ionic strength, also informs selection, as well as how the sample is fluidically delivered to the channel. In other words, the invention tolerates a range of channel geometry, with a range of thicknesses, surface area, length and width.

During use with the sample solution, a Debye length at the surface of the crumpled geometry is greater than a Debye length of an equivalent channel having a flat geometry. Because the Debye length is solution-dependent, the increase may be described in terms of a percent increase. For example, the crumpled geometry may be described as providing an at least 10%, 20%, 50% or 100% increase in Debye length compared to an equivalent uncrumpled or flat geometry for an equivalent sample.

The biosensor is compatible with any fluid sample. For example, the sample may comprise a biomolecule selected from the group consisting of a protein, a DNA sequence, an RNA sequence, and any fragments thereof, and the biological solution is unprocessed whole blood, plasma, saliva or sputum. “Unprocessed” refers to there is not any active steps to concentrate the target molecule.

The crumpled geometry may correspond to a multi-axial deformation or a uniaxial deformation of the channel layer. For example, an underlying support substrate may be corresponding deformed to shrink in a preferred direction, thereby resulting in spatially aligned peaks in the channel. Alternatively, an underlying support substrate may be corresponding deformed to shrink in all directions, thereby resulting in non-spatially aligned peaks in the channel.

The crumpled geometry may have an average periodicity ranging from between 1 nm and 100 nm and an average amplitude ranging from between 1 nm and 100 nm.

The channel layer may be in continuous contact or discontinuous contact with a support substrate layer.

The biosensor electrode separation distance may be between 1 μm and 5 cm.

The support substrate can be formed of a material capable of undergoing a shrinkage transformation to thereby crumple the channel layer that is supported by the support substrate. The shrinkage may be by an applied force that is subsequently removed, to cause a strained support layer to decrease in size and relax to an unstrained dimension, thereby crumpling the channel bonded to the support layer, including discretely bonded. The shrinkage may be induced by a temperature change.

Also provided herein are methods of using any of the biosensors described herein. For example, the method may be for detecting a biomolecule by the steps of providing the biosensor and introducing the sample solution to the sample reservoir channel layer. A gate voltage is applied to the sample solution. A FET electrical parameter is monitored, wherein a change in the FET electrical parameter corresponds to presence of the biomolecule in the sample solution. Examples of FET electrical parameters include, capacitance, electric potential, resistivity, current and the like.

The crumpled geometry can be selected to provide an increase in sensitivity is by a factor of at least 1000× compared to a conventional sensitivity for an equivalent planar channel geometry. The increase in sensitivity can be further achieved by providing a microfluidic system to precisely position the sample at the channel surface, and also by use of probes that selectively bind the target molecule. Accordingly, any of the methods may further comprise the step of anchoring a probe to the channel, wherein the probe has a capture sequence configured to selectively bind a target biomolecule of interest.

In an embodiment, the Debye length for a flat FET channel may be less than the thickness of the source and drain electrodes. The geometry of the crumple may be selected to extend the Debye length that is greater than the source and drain electrode thickness, thereby effectively increasing the distance at which a charged molecule can be reliably detected more into the bulk solution volume.

The method may be configured for micro RNA (miRNA) detection, cell-free DNA (cfDNA) detection, protein detection using an antibody capture agent, or small molecule detecting using an aptamer capture molecule. The method is compatible for use with a sample solution corresponding to an unamplified sample.

The method may be configured for detecting DNA, RNA, including miRNA, protein, or a small molecule circulating in plasma or whole blood.

Also provided are specially configured FETs and related methods useful in a range of applications, including for determining presence or absence of a target polynucleotide in a sample. In particular, the FET channel is specially configured so that ssDNA preferably interacts with the channel surface relative to dsDNA. In this manner, an amplification solution plus sample that may contain an amplifiable target polynucleotide of an amplified polynucleotide sequence, is introduced to the FET, and whether or not a target polynucleotide is present in the solution is determined by monitoring a FET parameter whose output is dependent on the amount of primer in the amplification solution. As target polynucleotide is amplified, the ssDNA primer is incorporated into an amplified dsDNA target, and so less ssDNA primer is available to interact with the FET surface. This is reflected by a change in a FET parameter from the original “baseline” value where all ssDNA primer is available to interact with the FET. As more ssDNA primer is “incorporated” into the amplified target, there is a greater deviation or difference from the initial baseline FET parameter. In this manner, the amount of target polynucleotide in the original sample can be determined. In contrast, if there is no polynucleotide target in the sample, the FET parameter is unchanged as the amount of ssDNA primer in the amplification solution is unchanged. In a particularly useful embodiment, the FET's described herein may be incorporated into the biosensors described herein, including for a FET having a crumpled channel material, such as a semiconductor material.

The FET preferably has a crumpled channel layer that is formed of a semiconductor material, including graphene. See, e.g., U.S. Pat. App. No. 62/982,801 filed Feb. 28, 2020 titled “ULTRASENSITIVE BIOSENSOR USING BENT AND CURVED FIELD EFFECT TRANSISTOR BY DEBYE LENGTH MODULATION” (Atty Ref. 338264: 7-20P US), which is specifically incorporated by reference for the devices disclosed therein, including crumpled or bent and curved FET, and related methods.

Any semiconductor material that can interact with the aromatic rings of a nucleotide molecule via pi-pi (π-π) interaction is suitable. One example is graphene, where the hexagonal cells of graphene and an aromatic ring of the ssDNA form noncovalent (e.g., “stacking”) π-π interaction that is significantly stronger than for a corresponding dsDNA. Other examples include, but are not limited to, MoS₂; dichalcogenides and silicene.

In an aspect, provided herein is a method of detecting amplification of a target polynucleotide. The method may comprise the steps of: providing a field effect transistor (FET) having a crumpled semiconductor material channel that is configured to form a π-π interaction with single stranded DNA; conducting an amplification reaction in an amplification solution comprising single stranded DNA (ssDNA) primers to obtain an amplified solution; contacting the FET with the amplified solution; and electrically detecting with the FET an amount of ssDNA primer in the amplified solution, thereby detecting amplification.

The semiconductor material is a two-dimensional layer selected from the group consisting of graphene; MoS₂; dichalcogenides and silicene. The two-dimensional aspect refers to a layer configuration to provide adequate receiving surface area for interactions with ssDNA. The material in the FET for interacting with ssDNA is preferably crumpled graphene, referred herein as a gFET.

The step of conducting the amplification reaction may occur prior to the contacting step or may occur simultaneously with the contacting step.

The method may further comprise the steps of: detecting a FET electrical parameter prior to the step of conducting the amplification reaction to obtain a baseline FET electrical parameter value; detecting the FET electrical parameter after the step of conducting the amplification reaction to obtain a post-amplification FET electrical parameter value; comparing the baseline and the post-amplification FET electrical parameter values; and identifying presence of the target polynucleotide for a statistically significant difference between the baseline and the post-amplification FET electrical parameter values. The concept of “statistical significance” is a recognition that there is some inherent variability in experimental systems, including in the biological context, including arising from noise or random perturbance in the system, including degradation of ssDNA in the sample for reasons unrelated to amplification. Depending on the application of interest, including starting materials, the amount of change in the FET electrical parameter may be set to a value that will encompass these variations, such as greater than a 5%, greater than 10% or greater than 25% difference, corresponding to an at least 5%, at least 10% or at least 25% decrease in ssDNA primer.

The FET electrical parameter may be a change in current at a fixed voltage or a Dirac point shift voltage.

The electrically detecting step may occur periodically or continuously during the step of conducting the amplification reaction.

The method is compatible with any of a range of initial starting concentration of the target polynucleotide, including as low as 8×10⁻²¹ M in the amplification solution. The actual detection sensitivity is affected by a number of parameters, including the primer sequence (e.g., homology to target sequence), length of time of amplification, solution composition or purity (e.g., amount of ssDNA that is not the ssDNA primer), and the like.

The negative and positive target polynucleotide amplification solutions can be distinguished from each other at a target polynucleotide detection limit of between 4×10⁻²¹ and 1×10⁻¹⁸ M.

As described hereinbelow, the ssDNA primer binds to a surface of the crumpled material (e g , graphene) by noncovalent π-π interaction between hexagonal cells of a crumpled graphene and an aromatic ring of the ssDNA and amplified dsDNA does not bind to the crumpled graphene as strongly as ssDNA due to π-π stacking of aromatic rings in the dsDNA.

The ssDNA primers may be provided at a concentration so that after 60-90 minutes of amplification, at least 90% of all available ssDNA primers have been incorporated into amplified dsDNA.

The methods provided herein are compatible without labels and/or without surface-functionalization. This makes the methods, and related systems that implement the method, robust, reliable, cost-effective and particularly well suited with point-of-care devices. Unlike conventional amplification assays, where amplified target is visualized, such as with a fluorescent probe, the instant amplified target is electronically detected. This avoids need for labels and associated optical components.

The methods provided herein are compatible with an amplification reaction that is a loop mediated isothermal amplification (LAMP or RT-LAMP) reaction or a polymerase chain reaction (PCR). A LAMP reaction is preferred as it avoids the need for continuous thermal cycling over given number of amplification cycles.

The target polynucleotide may be present in the amplification reaction so that ssDNA primers are incorporated into amplified double stranded DNA (dsDNA) amplification product and identification of target polynucleotide comprises identifying a change in a FET electrical parameter measured during the electrically detecting step, including a decrease in a Dirac point shift, including a change in FET electrical parameter that is statistically significant from a baseline value.

Similarly, the target polynucleotide may be absent from the amplification reaction so that ssDNA primers are not incorporated into an amplified double stranded DNA (dsDNA) amplification product and identification of no target polynucleotide comprises identifying a no change condition in a FET electrical parameter measured during the electrically detecting step, including a no change in a Dirac point shift, including a not statistically significant change in the FET electrical parameter.

The methods and systems provided herein are compatible with a range of applications. For example, the target polynucleotide may be associated with: a pathogen; a disease condition; a genetic mutation; or a gene of interest. The pathogen may be viral or bacterial.

The methods, being label free, robust and sensitive, may be incorporated in a point-of-care device for rapid target polynucleotide identification in less than 1 hour, including as fast as 10 minutes or less.

Also provided herein are systems that implement any of the methods described herein.

Provided are systems for detecting a target polynucleotide in a sample solution comprising: a FET having: a source electrode; a drain electrode, wherein the source and drain electrodes are separated from each other by an electrode separation distance; a channel layer between the source electrode and the drain electrode, wherein the channel layer comprises a two-dimensional crumpled semiconductor material; a channel layer receiving surface that forms part of a sample reservoir, wherein the sample reservoir is configured to hold an amplifiable sample solution; an electrical detector electrically connected to the FET. See, e.g., U.S. Pat. App. No. 62/982,801 filed Feb. 28, 2020 for various FET configurations having a crumpled material therein, which is specifically incorporated by reference herein. The amplifiable sample solution comprises ssDNA primers and amplification reagents to amplify the target polynucleotide, wherein during use the amplifiable sample solution contacts the channel layer receiving surface of the sample reservoir. The ssDNA primers during use bind to the channel layer receiving surface by a noncovalent π-π interaction between the crumpled semiconductor material and an aromatic ring of the ssDNA at a higher affinity than dsDNA, and the electrical detector is configured to detect a level of ssDNA primer by detection of a change in a FET electrical parameter.

The crumpled semiconductor material is selected from the group consisting of: graphene; MoS₂; dichalcogenides and silicene.

The FET electrical parameter may be a Dirac point shift voltage or a change in current at a fixed voltage, wherein the magnitude of the change varies with amount of ssDNA probe in the amplifiable sample solution.

The devices (biosensors) and methods provided herein are compatible with a range of to-be-detected targets or analytes, including charged or uncharged targets or analytes. For example, for a device that is a biosensor, the biosensor may detect a biomolecule, such as a polynucleotide, protein, antibody, polypeptide, and fragments thereof, as well as small molecules. The target may be a biomarker, including a biomarker associated with an infectious agent, thereby providing a diagnostic platform. For example, COVID-19 viral proteins are detectable, including capsid and spike protein, indicating the biosensors provided herein are useful for diagnosing infection with an infectious agent, including a virus or a bacteria. Uncharged molecules may also be detected, as illustrated by the experimental results for dopamine detection. Proteins, in general, are detected, as evidenced by the experimental results for IL-6 Accordingly, the biosensors provided herein are a useful platform for detecting a wide range of biomolecules.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1G: Scheme and characterization of flat and crumpled graphene FET biosensor. FIG. 1A. Cross-sectional scheme of the flat (left) and crumpled (right) graphene FET DNA sensor. Probe (black) and target (red) DNA strands are immobilized on the surface of graphene. The blue dot lines represent Debye length in the ionic solution and the length is increased at the convex region of the crumpled graphene, thus more area DNA is inside the Debye length, which makes the crumpled graphene more electrically susceptible to the negative charge of DNA. The inset boxes represent qualitative energy diagram in K-space. Graphene does not have intrinsic bandgap. However, crumpled graphene may open bandgap, which is discussed below and in FIG. 28. FIG. 1B. Fabrication of FETs and experimental process flow. Graphene on pre-strained PS substrate was annealed at 110° C. to shrink the substrate and crumple the graphene. Then source and drain electrodes were applied and solution-top gate was used. In case of flat graphene FET, the annealing process was omitted. FIG. 1C. SEM images of crumpled graphene. The scale bar is 5 μm (left) and 500 nm (right). FIG. 1D. Raman spectroscopy of crumpled graphene and PS substrate. FIG. 1E. Charge transfer characteristics of the fabricated crumpled graphene FET. V_{gs} versus I_{ds} (bottom) with the variation of V_{ds} graphs showed shift in the Dirac point. FIG. 1F. Dirac point shifts of the FET sensor plotted as a function of pH values. n=5, mean±std. FIG. 1G. Current-voltage plots for various gate voltages.

FIGS. 2A-2I. Nucleic acids absorption and hybridization test on flat and crumpled FET. FIG. 2A. Lateral image of the flat (top) and crumpled (bottom) graphene FET DNA sensors. DNA (red strand) is absorbed on the graphene surface by a-t stacking. FIG. 2B. I-V relationship of the flat (top) and crumpled (bottom) graphene FET sensors for the DNA absorption. DNA absorption shifted the I-V curve according to the indicated concentrations. The I-V curves shift of crumpled graphene is significantly larger than the flat device. FIG. 2C. Dirac voltage shift of the FET sensor. The Dirac voltage shift is plotted as a function of the added target DNA concentration. FIG. 2D. Lateral image of the flat (top) and crumpled (bottom) graphene FET DNA sensors. DNA (red strand) is hybridized with probe DNA (black strand) on the graphene surface. FIG. 2E. I-V relationship of the flat (top) and crumpled (bottom) graphene FET sensors for the DNA hybridization. DNA hybridization shifted the I-V curve according to the indicated concentrations. The I-V curves shift of crumpled graphene is significantly larger than the flat device. FIG. 2F. Dirac voltage shift of the FET sensor with detection of hybridization using DNA probe. NC is non-complementary control sequences used in the experiments. FIG. 2G. Sips model fitting results Y-axis is absolute values of Dirac point shift. FIG. 2H. Dirac voltage shift of the FET sensor with detection of hybridization using PNA probe. FIG. 2I. Dirac voltage shift of the FET sensor with miRNA detection of hybridization. Target RNA spiked in human serum was treated on the FET sensor. Human serum is complex mixture of biological components. The DNA and RNA sequence used in the experiments is shown in Table 1. All the data points are obtained from three different devices. mean±std. *P<0.05.

FIGS. 3A-3K. The schematic of the simulations for equilibrated DNA on (FIG. 3A) flat graphene; (FIG. 3B) concave surface of crumpled graphene; (FIG. 3C) convex surface of crumpled graphene; and (FIG. 3D) across the graphene crumples. Graphene is shown in blue, ions are presented as cyan and yellow spheres and the DNA bases are shown in different colors. Water molecules are not shown for better presentation. The molar concentration of ions (sodium and chloride) and the backbone of DNA strand along with the screening factor of ions are plotted as a function of the distance from the graphene surface for (FIG. 3E) flat; (FIG. 3F) concave; (FIG. 3G) convex; and (FIG. 3H) across configurations of DNA. The location where the ionic screening starts to take place is shown using an arrow. In the concave region, ions are excluded due to its confinement and most of the adsorbed DNA molecule remains unscreened electrostatically. Less screening increases N_{DNA}^{unscreend} and induces more charge density in graphene resulting in a larger Dirac point shift. FIG. 3I. The 2.45nm-diameter CNT that is used to model a narrow trench in crumpled graphene is shown with CNT and graphene carbon atoms in blue, ions in cyan and yellow, water molecules in red and DNA strand bases in different colors. The DNA adsorbs to the bottom of the trench and excludes ions near the surface (maximizing N_{DNA}^{unscreend}). The resulting giant electric potential modifies the carrier charge density of graphene. The potential is obtained from

$$V ( z ) = - \int {\int }_{{Z}_{0}}^{Z} \frac{q ( z )}{A {ε}_{0}} dz dz ,$$

whnere q(x), A and ε₀ are the net charge of the system (ions, DNA and water) in z, surface area of the bottom of the trench and vacuum dielectric constant, respectively. FIG. 3J. Electric potential as a function of distance from graphene for various geometry (flat, convex, concave). FIG. 3K. Impact of channel geometry on normalized electric potential.

FIGS. 4A-4H. Capacitance measurement and charge layer distance effect. FIGS. 4A-4D. The molar concentration map of ions (sodium and chloride) are plotted for flat and crumpled charged graphene sheet. The counter-ions are distributed over a longer distance away from the surface of graphene in the concave region of the crumpled graphene. FIG. 4E. EDL capacitance of flat and crumpled graphene. As EDL of the flat graphene is denser than the crumpled graphene, flat graphene had about 3 times larger capacitance than crumpled graphene. FIG. 4F. ELD structures of flat (left) and crumpled graphene (right). Loosely structured EDL of crumpled graphene leads to the smaller capacitance value. FIG. 4G. To determine if the crumpled graphene device has longer Debye length, distance of double strand (probe+target) DNA part was 3 nt further from the surface. FIG. 4H. The flat graphene device (blue line) is not able to measure the 19 nt (3nt short) target DNA while, the crumpled graphene (the orange line) showed left shift of IV curves. n=3, mean±std.

FIG. 5. The absolute value of the Dirac point shift dataset plotted as circular solid markers (top-most solid line for crumpled and bottom-most solid line for flat). The Sips fitting lines are shown as solid lines. The dotted line is the detection baseline that is estimated from the average value of the negative binding measurements shown in the main dataset figure.

FIG. 6. Bound concentration to graphene sensor as a function of incubation time, with the arrow indicated 1 hour incubation time and corresponding 16.5% target DNA concentration molecules bound to the surface.

FIGS. 7A-7E. Results of Raman spectroscopy analysis on VHB substrate.

FIGS. 8A-8C. Optical image of the crumpled graphene FET. FIG. 8A. Top view of the FET. S is source electrode and D is drain electrode made by silver paste. PS is polystyrene substrate. The liquid chamber was created using a silicone rubber. Graphene channel is indicated with red arrow. FIG. 8B. Lateral view of the device with 50 μl of PBS solution droplet. FIG. 8C. Lateral view of the device with 50 μl of PBS solution droplet after 1 hour in the probe station. ˜18 μl of water was dried thus the size of the droplet became smaller.

FIGS. 9A-9B. AFM images of flat and crumpled graphene transistor surface with and without the DNA immobilization. FIG. 9A. Left, AFM imaging and phase image of flat graphene surface in air showed mostly flat surface with some wrinkles. Right, flat graphene surface covered with DNA in air. The strands produce features polygonal structure. FIG. 9B. Left, AFM imaging and phase image of crumpled graphene surface in air. Increased crumple height/roughness is observed. Hierarchical wrinkling is clearly evident. Right, in AFM image, significant difference was not observed before and after DNA functionalization. However, phase imaging showed polygonal structure which was similar to the DNA features on flat graphene. For AFM imaging, 47 nt of partially double stranded DNA was used (Table 1). All images have a scan area of 1×1 μm².

FIGS. 10A-10F. Characterization of flat and crumpled graphene FET. FIGS. 10A-10B. The Raman D-to-G peak of the flat and crumpled graphene had similar intensity ratio. The background Raman spectrum of PS substrate is shown. FIGS. 10C-10F. Charge transfer characteristics of the fabricated FET using fluid gate. FIGS. 10C-10D. Both V_{ds} versus I_{ds} with the variation of V_{g} showed slightly curved relationship due to Dirac point. FIGS. 10E-10F. V_{g} versus I_{ds} with the variation of V_{ds} graphs also showed shift in the Dirac point.

FIG. 11. Sheet resistance measurement by Van der Pauw method. Using the equation from reference paper (Chin. Phys. B Vol. 26, No. 6 (2017) 066801). The sheet resistance is ˜450Ω, which is in concordance with known values.

FIGS. 12A-12B. Dirac voltage point stability test. The I-V measurements were repeated over time to evaluate the Dirac voltage point (the gate voltage at the minimum drain current point) was stable. FIG. 12A. The I-V curve of the probe functionalized device was measured in 1× PBS at the given time in the legend. FIG. 12B. The device was rinsed with fresh PBS buffer solution at points marked with red dot line. The Dirac voltage values were stable and did not showed shift. The increment of the gate voltage was 2 mV.

FIGS. 13A-13C. pH responses of flat and crumpled FETs. The measurements were performed in a PBS buffer from pH 3 up to 11 at constant drain-source voltage of 0.05V. The pH of PBS was adjusted by adding hydrochloric acid (HCl) or sodium hydroxide (NaOH). FIG. 13A. I-V relationship of the flat (top) and crumpled (bottom) graphene FET sensors at different pH values. FIG. 13B. Dirac point shifts of the FET sensor plotted as a function of pH values. The average values of Dirac point shift with pH were 12.2 mV/pH for flat FET and 27.2 mV/pH for crumpled FET. n=5, mean±std. FIG. 13C. The molar concentration difference of OH⁻ and H⁺ as a function of the distance from the positively charged graphene surface for flat, convex and concave cases. A much higher negative charge concentration is observed for the concave regions. This higher charge concentration induces a stronger change in charge carrier density of graphene resulting in a larger Dirac point shift in experiments.

FIG. 14. Scheme of probe DNA immobilization process. Probe DNA has a primary amino group positioned at the 5′-end with a standard (C6) spacer arm (top). The amino group is covalently reacted with one side of PASE molecules (middle). The other side of PASE is π-π stacked with graphene (bottom).

FIG. 15. P-value of Dirac point shifts between crumpled and flat graphene FET biosensor at 20 aM of target DNA hybridization. The graph shows clear difference between two data points with p-value of 0.0135.

FIG. 16. Schematic of the molecule with the DNA (SEQ ID NO:1) and PNA (SEQ ID NO:2) sequence and linker molecule structure. Left, the structure that was used for the DNA/DNA hybridization experiments. Right, the structure that was used for the PNA/DNA hybridization experiments. PNA has 7 carbon shorter distance between nucleic acids and graphene surface.

FIGS. 17A-17B. Quantification of DNA using radioactive labeling. Radioactive isotope phosphorus 32 (P32, yellow star) was labeled at the end of target (red) and probe (black) DNA. FIG. 17A. target DNA was absorbed on both flat and crumpled graphene by π-π stacking and quantified. FIG. 17B. probe DNA was immobilized on both flat and crumpled graphene. All the experiment conditions were same with FIGS. 2A-2I. The number represent the relative intensity normalized with respect to the third image (1.00). The results show that density of DNA on the flat and the crumpled graphene is in the same order.

FIG. 18. The interaction energy between graphene and DNA strand as a function of simulation time for the different configurations of DNA on the crumpled graphene as well as on flat graphene.

FIG. 19. The packing of DNA molecules adsorbed onto the graphene surface. The area per nucleotide is plotted along the y-axis if the entire DNA concentration were to be adsorbed as a single layer on the graphene surface. The area occupied by one DNA nucleotide is assumed to be ˜8 nm² based on DNA nucleotide size (the dashed line). Concentrations higher than 10⁻⁹ M results in extreme packing (area per nucleotide smaller than nucleotide size) which indicates saturation of a monolayer of DNA molecules on the graphene surface.

FIG. 20. AFM image of uniaxially crumpled graphene. The white arrows indicate fine crumples with a few nanometer sizes.

FIGS. 21A-21D. Percentage of DNA charge transfer to graphene to match the experimental Dirac point shift (FIG. 21A) without band gap contribution, and (FIG. 21B) with band gap contribution. Without band gap, the required charge transfer exceeds the maximum available limit indicating that charge transfer is not the only mechanism by which Dirac point shift occurs. FIG. 21C. Contribution of charge transfer and band gap (a constant Dirac shift due to band gap is assumed) to the total Dirac point shift. FIG. 21D. Three different regions are defined. <200 aM region where the band gap opening is dominant, >200 aM and <1 nM region where the charge transfer becomes significant in addition to the band gap opening and >1 nM region where the charge transfer is dominant while DNA adsorption saturation on graphene takes place.

FIGS. 22A-22E. The modelled I-V curves before and after adding 2 fM complementary DNA for (FIG. 22A) flat and (FIG. 22B) crumpled graphene. The I-V curves before and after adding 2 aM complementary DNA for (FIG. 22C) flat and (FIG. 22D) crumpled graphene. The potential shift in FIGS. 22A-22D is computed assuming 80% (for flat graphene) and 10% (for crumpled graphene) ionic screening using

$$Δ {V}_{D} = \frac{e {N}_{D N A}^{unscreened}}{{C}_{T}} .$$

The Dirac point shift due to a bandgap opening in 10⁻⁷% of the crumpled graphene after adding 2 aM complementary DNA is ˜5 mV and the corresponding I-V curve is plotted in FIG. 22E.

FIG. 23. Bandgap as a function of the size of graphene unit cell for a single DNA base. The bandgap is normalized by the bandgap of 1-unit cell. The width of all the cells is ˜12 Å and only the length is varied. The graphene bandgap in 4-unit cell is about 60% of that of 1-unit cell which is still a significant bandgap value. This shows that the effect of a single DNA base on the bandgap of graphene is long range.

FIGS. 24A-24C. The nucleobase orientations above the pristine graphene surface used in the DFT/GW simulations. FIG. 24A. Orientation 1, where the plane of the base ring is parallel to the a-b plane. FIG. 24B. Orientation 2, where the plane of the base ring is perpendicular to the a-b plane (or parallel to the a-c plane). FIG. 24C. Orientation 3 is similar to orientation 2 where the ring is rotated 180 around the a-axis.

FIGS. 25A-25C. The nucleobase orientations above the crumpled graphene surface used in the DFT/GW simulations. FIG. 25A. Orientation 1, where the plane of the base ring is parallel to the a-b plane. FIG. 25B. Orientation 2, where the plane of the base ring is perpendicular to the a-b plane (or parallel to the a-c plane). FIG. 25C. Orientation 3 is similar to orientation 2 where the ring is rotated 360° around the a-axis.

FIGS. 26A-26D. Interaction of the nucleobase with the crumpled graphene surface for different orientations: (FIG. 26A) interfacial charge density for parallel orientation (orientation 1) and (FIG. 26B) interfacial charge density for perpendicular orientation (orientation 2) (green and red colors represent negative and positive charge difference, respectively). Local potential versus the distance from the bottom of the crumpled graphene, (FIG. 26C) for orientation 1 and (FIG. 26D) for orientation 2 along the c-direction (red curve is the total electrostatic potential and blue curve is the Hartree contribution).

FIGS. 27A-27B. Partial density of states for: (FIG. 27A) pristine graphene and (FIG. 27B) crumpled graphene for different molecular orbitals. Total density of states (gray filled), s-orbital contribution (red), px-orbital contribution (blue), py-orbital contribution (green), and pz-orbital contribution (orange) are shown.

FIG. 28. The calculated band gap (in eV) of graphene in the presence of single DNA bases with three different orientations using DFT and GW. The GW values are reported in parenthesis. The crumpled graphene has a wavelength of 0.818 nm and an amplitude of 0.26 nm. See FIGS. 24A-24C and 25A-25C for the schematic of the orientations.

FIGS. 29A-29D. EDL capacitance of graphene. FIG. 29A. The capacitance measurement of flat graphene. FIG. 29B. The capacitance measurement of crumpled graphene. The capacitance of flat graphene was about 3 times larger than the capacitance of crumpled graphene. FIGS. 29C-29D. Capacitance measurement with atomic layer deposition (ALD). 5 nm of Al2O3 was deposited on both flat and crumpled graphene by (ALD) to reduce the leakage current. Flat graphene still showed about 4 times larger capacitance crumpled graphene. Both experiments showed that most of the capacitance change was attributed to the modulated screening length due to the nanoscale morphology.

FIG. 30. Dirac point shift with four different concentrations of PBS buffer solution. n=5, mean±std.

FIGS. 31A-31B. I-V curve of charge layer distance experiment. FIG. 31A. While flat graphene FET does not show significant Dirac point shift. FIG. 31B. Crumpled graphene FET showed left shift of IV curves. It supports the hypothesis of Debye length or EDL length change crumpling the graphene into nanoscale morphology.

FIG. 32. Schematic of the molecule with the DNA sequence and linker molecule structure. Left, the structure that was used for the hybridization experiments. Right, 3 nt was removed for the charge layer distance experiment. For both cases, DNA is linked to graphene with 12 carbon linkers.

FIG. 33A. FET Sensor with a biosensing unit cell. FIG. 33B is a cross-section of the biosensor of FIG. 33A. For clarity, the channel layer is illustrated as flat.

FIGS. 34A-34B. Electrical sensing on flat (FIG. 34A) and curved (FIG. 34B) sensors.

FIG. 35. Fabrication and functionalization of the crumpled FETs.

FIG. 36: Detecting enzymatic DNA amplification. Process overview of detecting target DNA using loop mediated isothermal amplification (LAMP) followed by detection of primer (ssDNA) on gFET sensors. Schematic of crumpled graphene FET DNA sensor and its adsorption of ssDNA and not dsDNA. LAMP was used to amplify target DNA causing consumption and decrease of primer concentration. Reduced primer concentration post amplification gives a lower Dirac voltage shift on the sensor.

FIG. 37A-37D: Characterization of the gFET and DNA adsorption studies. FIG. 37A: Microscopic optical image of the device. S and D indicate the source and drain contacts, respectively. Graphene channel is visible as shaded region in the middle. FIG. 37B: Phase image of the crumpled graphene clearly shows the nanoscale crumpling features on the graphene surface. Wrinkles as small as a few hundred nanometers can be seen. FIG. 37C: Dirac voltage shift of the FET sensor for ssDNA adsorption on flat and crumpled graphene (n=3). FIG. 37D: Dirac voltage shift of the crumpled graphene FET sensor for dsDNA adsorption test. The Dirac point shift even for the highest concentrations of dsDNA (˜0 mV for 2 uM dsDNA) is small in comparison to even very low concentrations of ssDNA (˜6 mV even for 2 aM ssDNA) (n=3).

FIG. 38A-38D: AFM images of flat graphene surface showing primer adsorption pre and post amplification. FIG. 38A: AFM image and phase image of bare graphene surface. FIGS. 38B-38C: AFM image and phase image of pre-amplified DNA (FIG. 38B) and post amplified DNA (FIG. 38C) on flat graphene surface. The phase images show a rough surface with black structures indicating pre-amplification ssDNA (primers) adsorption to the graphene surface. Post amplification, concentration of dsDNA is greater, which cannot be adsorbed on the graphene surface. Phase images show smoother surface with less black structures. FIG. 38D: AFM image and phase image of 1 uM dsDNA on flat graphene surface, which show that dsDNA cannot be highly adsorbed on the graphene surface.

FIGS. 39A-39E: Attomolar E. coli DNA detection using crumpled graphene FET biosensors. FIG. 39A-39B: Raw fluorescence data and amplification thresholds for different concentrations of E coli DNA (n=3). DNA concentrations from 4 aM to 40 fM were amplified over a 60 min LAMP reaction with 1× primer concentration per reaction (0.15 μM of F3 and B3, 1.17 μM FIP and BIP, and 0.59 μM of LF and LB primers. Only 1 out 3 repeats for 4 aM DNA concentration amplified. Both amplified and non-amplified DNA was measured for Dirac Point Shift. FIG. 39C: Absolute value Dirac Point Shifts for primer (negative control) and amplified dsDNA samples on three different FET devices. 1:100 dilution in 1× PBS of all post-reactions samples was made for Dirac voltage measurements. The shift for 5 amplified samples is lesser (8.4-10 mV) than that for 5 non-amplified samples (negative control and positive time zero samples). FIG. 39D: Normalized Dirac Point Shifts (n=3) for each sample of the three devices in FIG. 39C. FIG. 39E: Box and whisker plot for Normalized Dirac Point Shift for amplified (40 fM-4 aM) and non-amplified (Negative and Positive controls t=0, Negative t=60, and unamplified 4aM) samples of the three devices in FIG. 39C. The error bars show the range of the normalized data.

FIGS. 40A-40D: Electrical detection of amplified ZeptoMolar concentrations of DNA on crumpled graphene FET sensors. FIG. 40A: Process flow of zeptomolar LAMP reactions and detection on crumpled graphene. In 1 mL of diluted sample containing zeptomolar concentrations of target DNA, lamp reagents such as primers and polymerase were added and the reaction was conducted at 65 C. Amplified sample was loaded and incubated on crumpled graphene, after which Dirac point measurements were taken. FIG. 40B: Absolute value Dirac point shift for primer (negative control) and amplified dsDNA samples on three different FET devices. Starting DNA concentrations from 8 zM to 400 zM were amplified over 60 min reaction with 1× primer concentration per reaction. Post reaction, 1:100 dilution in 1X PBS of all samples was made for Dirac voltage measurements. The shift for the 3 amplified samples is lesser (5.4-12.7 mV) than that for non-amplified samples (2 measurements of negative control). FIG. 40C: Normalized Dirac Point Shifts (n=3) for each sample of the three devices in FIG. 40B. FIG. 40D: Box and whisker plot for the Normalized Dirac Point Shift for amplified (40 fM-4 aM) and non-amplified (Negative and Positive controls t=0, Negative t=60, and unamplified 4 aM) samples of the three devices in FIG. 40C. The error bars show the range of the normalized data.

FIGS. 41A-41D: Primer and amplified DNA adsorption test on crumpled FET (1e5 dilution of sample for Dirac Point measurement). FIGS. 41A-41BD: Raw fluorescence data and amplification thresholds for different concentrations of E coli DNA (n=3). Starting DNA concentrations from 4 aM to 40 fM were amplified over a 60 min LAMP reaction with 1× primer concentration per reaction. Only 1 out 3 repeats for 4 aM DNA concentration amplified. Both amplified and non-amplified DNA samples were measured for Dirac Point Shift. FIG. 41C: Absolute value Dirac Point Shifts for primer (negative control) and amplified dsDNA samples on two different FET devices. 1:1e5 dilution in 1× PBS of all post-reactions samples was made for Dirac voltage measurements. Device 1 40 fM, 40 aM, and amplified 4 aM samples did not show a shift. The shift for 5 amplified samples is less (6.8-8.4 mV) than that for 5 non-amplified samples (negative control and positive time zero samples). The shift of non-amplified samples with 1:1e5 dilution is lesser (6.4 mV) than that for 1:100 dilution. FIG. 41D: Box and whisker plot of Normalized Dirac Point Shift for amplified (40 fM-4 aM) and non-amplified (Negative and Positive controls t=0, Negative t=60, and unamplified 4 aM) samples of the two devices in FIG. 41C. The error bars show the range of the normalized data.

FIGS. 42A-42D: Primer and amplified DNA adsorption test on crumpled FET (1e4 dilution of sample for Dirac Point measurement). FIGS. 42A-42B: Raw fluorescence data and amplification thresholds for different concentrations of E coli DNA (n=3). Starting DNA concentrations from 4 aM to 40 fM were amplified over a 60 min LAMP reaction with 1× primer concentration per reaction. Only 1 out 3 repeats for 4aM DNA concentration amplified. Both amplified and non-amplified DNA samples were measured for Dirac Point Shift. FIG. 42C: Absolute value Dirac Point Shifts for primer (negative control) and amplified dsDNA samples. 1:1E4 dilution in 1× PBS of all post-reactions samples was made for Dirac voltage measurements. The shift for 5 amplified samples is less (6.8 mV) than that for 5 non-amplified samples (negative control and positive time zero samples). The shift of non-amplified samples with 1:1E4 dilution is less (4.6 mV) than that for 1:100 dilution. FIG. 42D: Box and whisker plot of Normalized Dirac Point Shift for amplified (40 fM-4 aM) and non-amplified (Negative and Positive controls t=0, Negative t=60, and unamplified 4aM) samples of the device in FIG. 42C. The error bars show the range of the normalized data.

FIGS. 43A-43D: Primer and amplified DNA adsorption test on crumpled FET (0.1× primer concentration). FIGS. 43A-43B: Raw fluorescence data and amplification thresholds for different concentrations of E coli DNA (n=3). Starting DNA concentrations from 4 aM to 40 fM were amplified over a 60 min LAMP reaction with 0.1× primer concentration per reaction. Only 2 out 3 repeats for 4aM DNA concentration amplified. Both amplified and non-amplified DNA samples were measured for Dirac Point Shift. FIG. 43C: Absolute value Dirac Point Shifts for primer (negative control) and amplified dsDNA samples on two different FET devices. 1:100 dilution in 1× PBS of all post-reactions samples was made for Dirac voltage measurements. The shift for 5 amplified samples is lesser (9.2-11.2 mV) than that for 5 non-amplified samples (negative control and positive time zero samples). FIG. 43D: Box and whisker plot of Normalized Dirac Point Shift for amplified (40 fM-4 aM) and non-amplified (Negative and Positive controls t=0, Negative t=60, and unamplified 4 aM) samples of the two devices in FIG. 43C. The error bars show the range of the normalized data.

FIGS. 44A-44C: Electrical detection for post-reaction amplified Zeptomolar concentrations of DNA on crumpled graphene FET sensors. FIG. 44A: Absolute value Dirac Point Shifts for primer (negative control) and amplified dsDNA samples on three different FET devices. Starting DNA concentrations from 8 zM to 400 zM were amplified over 60 min reaction with 0.1× primer concentration per reaction. Post reaction, 1:100 dilution in 1× PBS of all samples was made for Dirac voltage measurements. The shift for 3 amplified samples is less (4-7.7 mV) than that for 2 non-amplified samples (negative control and positive time zero samples). FIG. 44B: Normalized Dirac Point Shifts (n=3) for each sample of the three devices in a. FIG. 44C: Box and whisker plot of Normalized Dirac Point Shift for amplified (40 fM-4 aM) and non-amplified (Negative and Positive controls t=0, Negative t=60, and unamplified 4 aM) samples of the two devices in FIG. 44A. The error bars show the range of the normalized data.

FIGS. 45A-45B: AFM images of bare flat graphene surface, dsDNA and ssDNA adsorption on graphene surface. FIG. 45A: AFM image (left) and phase image (right) of crumpled graphene surface in imaged air. FIG. 45B:AFM image (left) and phase image (right) of 1 uM ssDNA on flat graphene surface. Flat graphene surface covered with ssDNA (1 uM) in air. The strands produce some features on the graphene surface showing the adsorption of ssDNA on the graphene surface.

FIG. 46: Schematics and characterization of crumpled graphene FET biosensor for targeted biomarkers. The top panels illustrate: (i) Scheme of crumpled graphene FET biosensor ‘S’ is source, ‘G’ is liquid gate and ‘D’ is drain electrodes. (ii) Dopamine detection with aptamer probe and (iii) Various proteins detection with specific antibodies on the crumpled graphene channels. (iv) Scheme and the lateral section of COVID-19 virus which show targeted viral proteins for the proposed antigen test with crumpled graphene FET. The middle and bottom panels are SEM images of flat and different crumpling ratios of graphene. SEM images of (i) flat graphene and (ii˜iv) with 10%˜60% of crumpling ratios. (v˜viii) SEM images of same samples of (i˜iv) with a larger scale. Scale bars and crumpling ratios are indicated on images.

FIG. 47: The molar concentration of ions (sodium and chloride) and the backbone of COVID-19 RNA strand segment along with the screening factor of ions are plotted as a function of the distance from the graphene surface for flat (top left panel), 10% (top right), 30% (middle left), 50% (middle right) and 70% (bottom left) crumpled graphene. The configurations of different graphene are schematically illustrated in the respective plots. In concave regions of crumpled graphene, ions are excluded due to its confinement and most of the adsorbed COVID-19 RNA molecule remains unscreened electrostatically. As the degree of crumpling increases, more of the RNA molecules is exposed to the graphene surface without being electrostatically screened by ions resulting in enhanced RNA detection. The bottom right panel is a summary table of the simulations.

FIGS. 48A-48D: Characterization of crumpling ratio effects. FIG. 48A EDL capacitance of the graphene samples with crumpling ratio of 10%, 30%, 40% and 60%. FIG. 48B Strain analysis via confocal Raman spectroscopy. Top left panel: Raw Raman spectrum of graphene with various crumpling ratios. Top right panel: 2D peak position map of graphene with various crumpling ratios. Scale bars: 1 μm. Bottom left panel: 2D Raw Raman spectrum. Bottom right panel: 2D peak position graph. FIG. 48C: AFM image of (i) 40% and (ii) 60% crumpled graphene. 3D AFM image of (iii) 40% and (iv) 60% crumpled graphene. FIG. 48D Dirac point shift by ssDNA absorption on graphene FET sensors with various crumpling ratios.

FIGS. 49A-49E: Dopamine detection with graphene FET biosensors. FIG. 49A Schematic of dopamine capturing with aptamer on the crumpled graphene channel. FIG. 49B I-V relationship of the crumpled and FIG. 49C flat graphene FET sensors for the dopamine detection using aptamer probe. FIG. 49D Dirac voltage shift of the FET sensor with detection of dopamine in PBS and FIG. 49E aCSF. NC is negative control samples which are non-specific biomolecules to the aptamer.

FIGS. 50-50D: Various protein detections on graphene FET biosensors. FIG. 50A Schematic of proteins captured with specific antibodies on the crumpled graphene channel. FIG. 50B Dirac voltage shift of the FET sensor with detection of IL-6 protein, FIG. 50C COVID-19 N-protein and FIG. 50D S-protein.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

In general, the terms and phrases used herein have their art-recognized meaning, which can be found by reference to standard texts, journal references and contexts known to those skilled in the art. The following definitions are provided to clarify their specific use in the context of the invention.

“Field effect transistor” (FET) refers herein to a transistor having a sensor that detects changes in an electric field in and around the sensor. One unique feature of the instant FETs is that the channel is formed of a layer (e.g., 2-D) that is in a bent and curved configuration, having peaks and troughs with a periodicity between peaks, that provides an increase in sensitivity. FETs are also referred herein generally as ion-sensitive FETs (ISFETs) to emphasize the FET is sensitive to ions in the sample. Provided herein are devices and methods that effectively extend the Debye length to minimize adverse impact of ions in the sample, so as to increase FET sensitivity and accuracy. Any of the FETs provided herein may correspond to an ISFET.

“FET electrical parameter” refers to an electrically-measured parameter such as current, voltage, impedance or a parameter calculated therefrom, that reflects a target molecule interaction with the FET sensor, including a ssDNA primer. The FET electrical parameter may be a Dirac point shift voltage.

“Physiological level” of salts refers to a solution that is isotonic relative to a biological material, so that the biological analyte does not adversely swell or shrink under osmotic pressure.

“Minimally processed” refers to a biological sample that may be provided to the devices herein without any complex processing steps and so may be suitable directly with a biological sample. One example of a process that is considered minimal is sample dilution by introduction of a physiologically-compatible fluid, such as a solution isotonic to biological materials suspended within the sample, such as PBS or equivalents thereof.

“Analyte” is used interchangeably with “target” and refers to a material suspended in a fluid to be detected by the FET sensor. In an aspect, the analyte is a biological material and so is suspended within a fluid having a relatively high ionic strength that is compatible with the biological material. As specifically exemplified herein the analyte or target may be a biomolecule, including a biomarker or a polynucleotide. The systems are exemplified herein for polynucleotides, proteins and small molecules. The biomolecule may be charged or uncharged. Particularly relevant biomolecules include those having a net charge. The devices and methods described herein, however, may be used to detect molecules that do not have a net charge. For example, any molecule that alters the electric potential underlying the bent channel. Crumpled biosensors can detect uncharged molecules using various underlying mechanisms, including: (1) An uncharged molecule binds to a charged receptor (protein, antibody, aptamer made of DNA, RNA, LNA) attached on the curved 2D sensor. The binding of the neutral molecule changes the conformation of the receptor molecule altering the charge state and the charged mirrored in the sensor layer. Due to the curved, bent and crumpled 2D layers, the sensitivity is enhanced as compared to a flat surface; (2) A molecule that has a net zero charge can have an internal dipole where the length of the dipole is on the order of the Debye length over the curved 2D sensor layer. In this case, the charge mirrored or imaged is perturbed and is detected in the sensor surface. In this manner, any net charged or even uncharged molecule such as a single nucleotide or amino acid, can be probed in the cavities within the crumpled, curved 2D sensor layer. These nanoscale cavities are ion free, and even water molecules can be excluded, so that even individual single bases or amino acids are detectable due to the different internal net charged state.

“Functionalized” is used broadly to refer to processing of the sensor or sensing surface to facilitate interaction or binding between a target and the sensing surface. The processing is dependent on the analyte being measured. For example, a capture agent, such as an antibody, a receptor, a polynucleotide, a polypeptide, or other target-specific material may be attached to the sensing surface to provide target-specific binding. The target-specific binding is determined by those of skill in the art based on binding kinetics between a capture agent and binding region of the target. For nucleic acid molecules, a sequence complementary to the binding region is used. For a protein, an antibody that specifically binds the protein may be used.

“Crumpled” refers to a spatially-varying height of the channel layer. In other words, the 2-D “flat” layer of material has a 3-D geometry provided by the bends and curves in the layer. The crumples may be formed as described in U.S. Pat. No. 9,908,285 (Three-Dimensional Texturing of Two-Dimensional Materials) by SungWoo Nam et al., which is specifically incorporated by reference herein, including for crumpled graphene/graphite.

A “π-π interaction” refers to the interaction between a ssDNA and a material of the FET and is also referred to as π-stacking or π-π stacking. It is a noncovalent interaction between aromatic rings. The interactions herein are much stronger for ssDNA than dsDNA, so that the electrical parameter from a FET having the material is used to assess how much ssDNA is present in the fluid. In this manner, determination of target polynucleotide is determined.

“Target polynucleotide” refers to a polynucleotide that is desired to be detected. As used herein, target polynucleotide is used broadly to refer to any polynucleotide sequence that is capable of being amplified by an amplification reaction using single stranded DNA (ssDNA) primers such that, in the presence of the target polynucleotide the ssDNA primers are incorporated into the amplified product, which is double stranded DNA (dsDNA). An example of a target polynucleotide is a molecule that is capable of being amplified, so that a target polynucleotide may be exponentially amplified by a technique such as PCR or an isothermal technique such as LAMP. “Template” refers to the nucleic acid that is to be amplified by PCR. The target may be DNA. In an aspect, the target to be amplified corresponds to a nucleic acid sequence that is uniquely identified with a specific organism. For example, to detect a bacterial pathogen, a target polynucleotide that is a contiguous nucleic acid sequence unique to that pathogen is selected. The polynucleotide sequence informs the selection of primers that provides specific amplification of the target polynucleotide. For example, the primer may target a portion of the polynucleotide sequence itself, or may target a flanking sequence to the target polynucleotide. Selection of primers based on the desired target sequence is known in the art, with primers generally about 15 to 30 nucleotides in length, having a high sequence “homology” to a target sequence (e.g., corresponding to G-C and A-T base pairing), with forward and reverse primers separated by suitable distance to ensure forward and reverse primers are incorporated into the amplified dsDNA amplification product.

“Amplification reaction” refers to the biological sample (in which a one or more target polynucleotide detection is desired) plus amplification solution. Accordingly, an “amplification solution” includes a buffer fluid comprising an amplification enzyme and primer(s) for nucleic acid amplification of a target polynucleotide sequence. The materials required may correspond to those necessary to perform a PCR as known in the art. Examples of such materials include primers, enzymes such as DNA polymerases (e.g., Taq. polymerase), reverse transcriptases, dNTP, nucleases, salts (MgCl₂) and PCR/LAMP buffers to facilitate effective PCR and LAMP. In an aspect, the amplification solution contains target polynucleotide, and more specifically a nucleic acid sequence that contains the target polynucleotide as well as flanking sequences. The solution may contain nucleic acid material from a biological cell, such as nucleic acid material from a lysed cell. In an aspect, the solution does not contain target polynucleotide, in which case the absence of target polynucleotide in the system will lead to no detection of target. The amplified product refers to the nucleic acid sequence that is produced as a result of the amplification reaction process, and corresponds to double stranded polynucleotides.

A solution or sample that “may contain target polynucleotide” refers to a sample that is introduced to the device in which it is desired to determine whether or not it contains target polynucleotide. For example, in point-of-care diagnostics or assays it is often desired to determine whether any of the DNA in the sample contains template. Similarly, in assays for the detection of a food borne pathogen, the target polynucleotide may correspond to a sequence that uniquely identifies the pathogen of interest. For rapid screening of multiple pathogens, the target polynucleotide may correspond to multiple target polynucleotides, with each target polynucleotide identifying a specific pathogen. Accordingly, the method may rapidly confirm there is no food borne pathogen, but if there is measureable detection, subsequent tests may be performed, if desired, to identify the specific pathogen.

“Isothermal amplification” refers to methods that can amplify nucleic acids exponentially, similarly to PCR amplification, but at a constant temperature, thereby avoiding need for thermocyclers. Examples include, but are not limited to, LAMP (loop-mediated isothermal amplification), HAD (helicase-dependent amplification), RCA (rolling circle amplification), MDA (multiple displacement amplification), WGA (whole gene amplification) and RPA (recombinase polymerase amplification).

The methods and devices provided are characterized as a platform-level technology in that the invention can be used with any target polynucleotide and, therefore, any primers, as known in the art of amplification reactions, such as polymerase chain reaction (PCR) and LAMP.

“Point-of-care” refers to tests performed on a sample obtained from a patient, wherein the diagnosis is provided at the time of the test. For example, tissue may be obtained directly from the patient, and introduced to any of the systems described herein, and a diagnostic result generated with the test result provided to the patient.

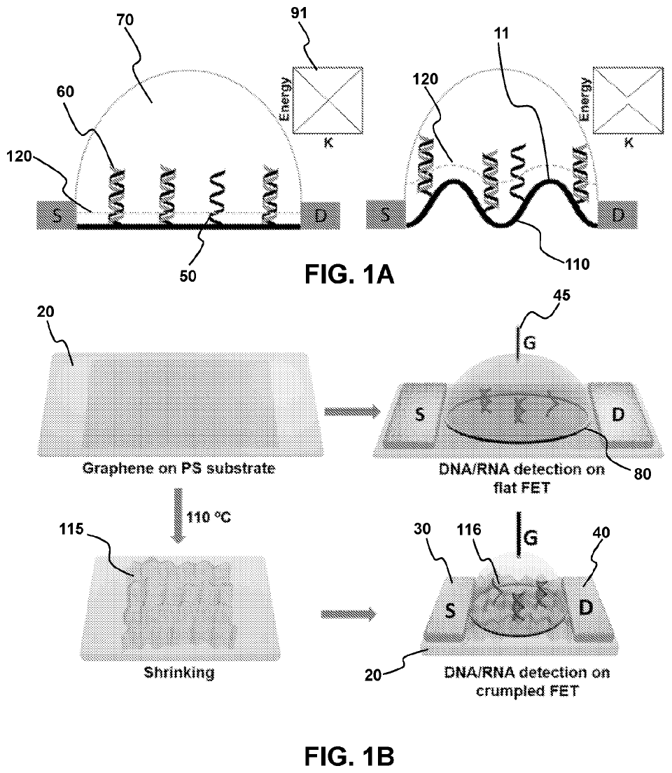

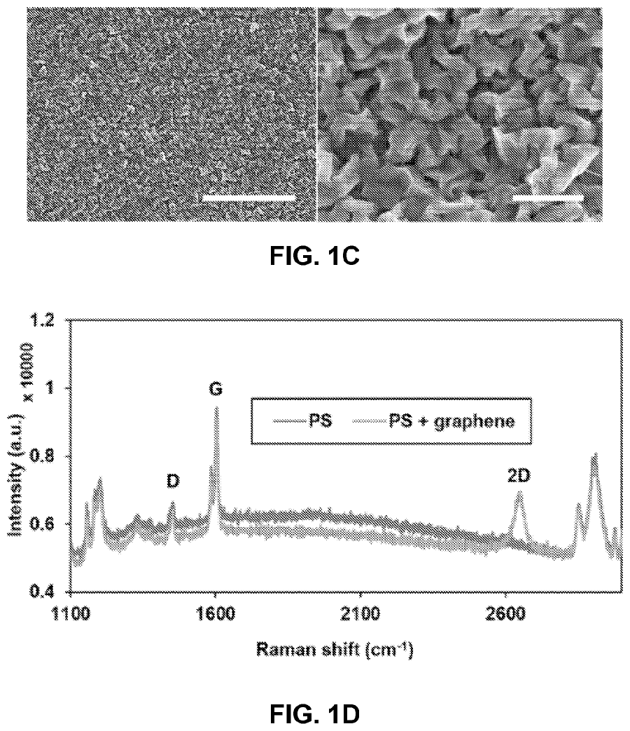

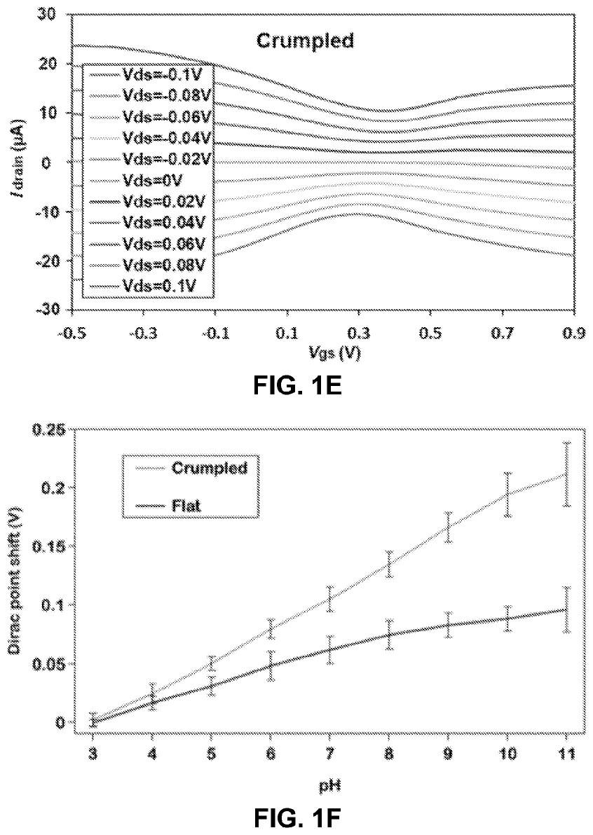

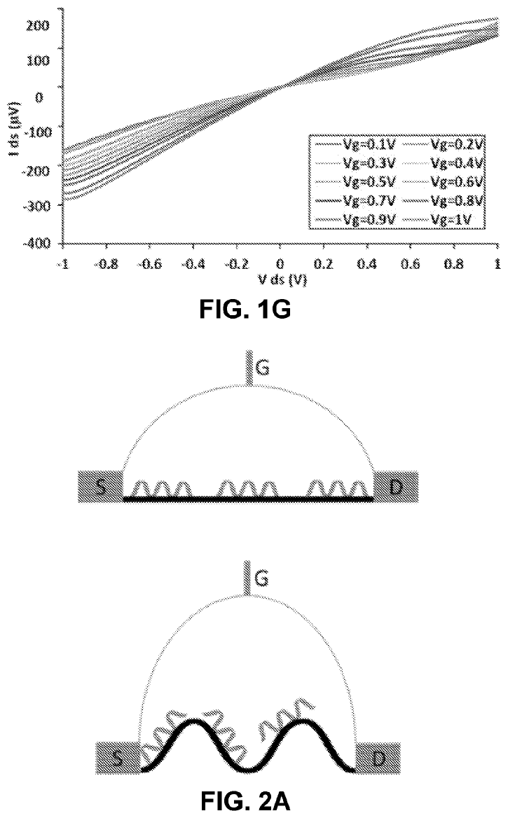

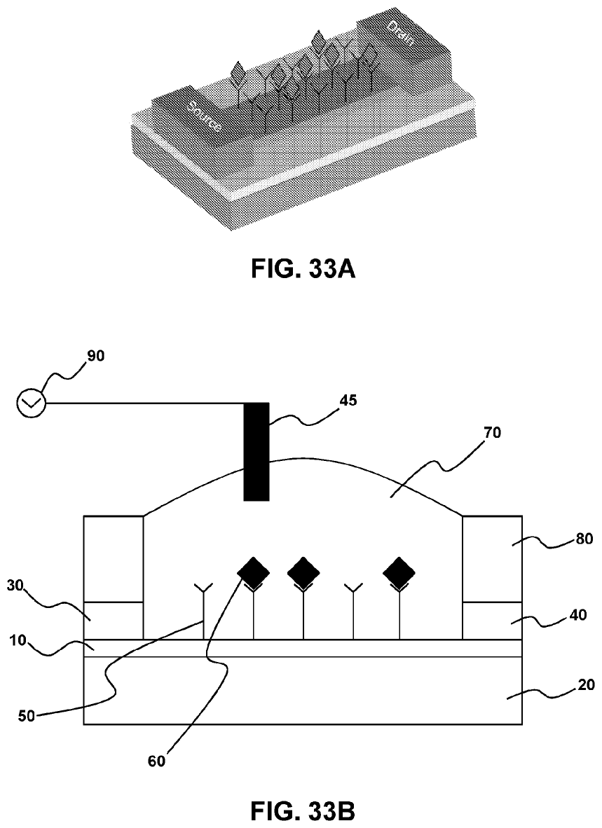

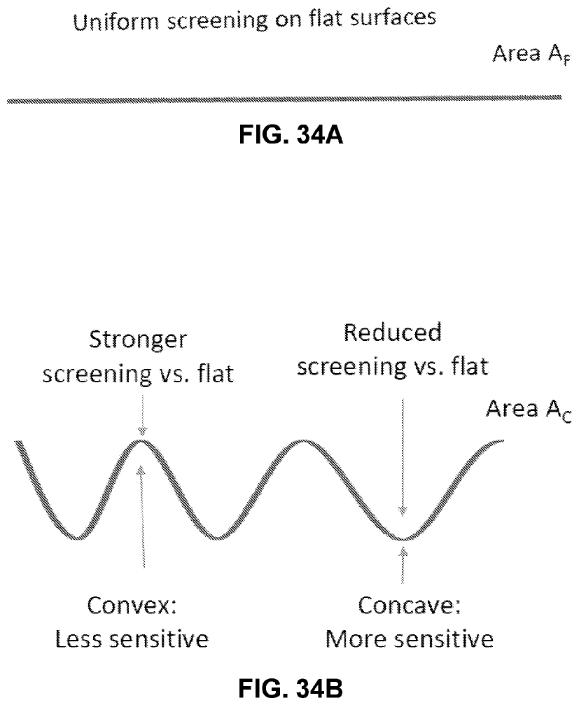

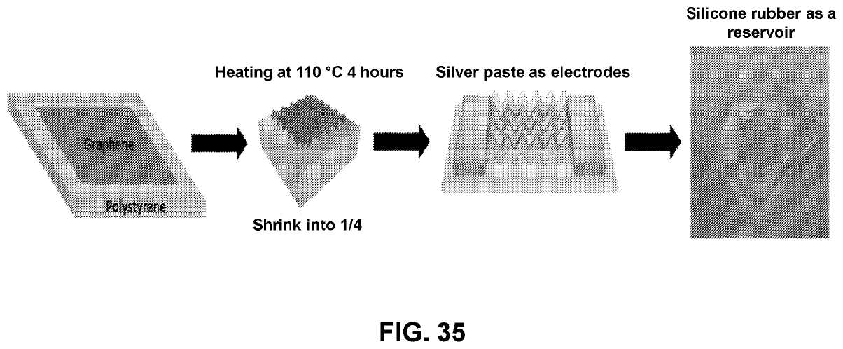

FIGS. 1A, 1B and 33B are schematic illustrations of a biosensor formed from a FET, with a channel layer 10 supported by a support substrate 20. Source 30 and drain 40 electrodes are separated from each other by an electrode separation distance. Gate electrode 45 electrically contacts the fluid sample solution 70 and connected to a power source 90. Channel layer 10 is positioned between the electrodes 30 and 40 and may have probes 50 extending from the channel layer surface. Probes 50 specifically bind a target molecule 60. Sample reservoir 80 is configured to hold sample solution 70. Referring to FIG. 1A, the right panel illustrates crumpled geometry 110, with the Debye length 120 that increases in the crumpled geometry relative to the conventional flat geometry. FIG. 1B illustrates support substrate 20 and a graphene layer having a crumpled geometry from a multi-axial 115 deformation of a substrate that undergoes a temperature-induced shrinkage transformation, thereby providing a channel with a multi-axial crumpled geometry 116 between source and drain electrodes 30 40.

The invention can be further understood by the following non-limiting examples.

### EXAMPLE 1

### Ultrasensitive Detection of Nucleic Acids Using Deformed Graphene Channel Field Effect Biosensors

Field-effect transistor (FET)-based biosensors allow label-free detection of biomolecules by measuring their intrinsic charges. The detection limit of these sensors is determined by the Debye screening of the charges from counter ions in solution. Here, we use FETs with a deformed monolayer graphene channel for the detection of nucleic acids. These devices with even millimeter scale channels show an ultra-high sensitivity detection in buffer and human serum sample down to 600 zM and 20 aM, respectively, which are ˜18 and ˜600 nucleic acid molecules. Computational simulations reveal that the nanoscale deformations can form ‘electrical hot spots’ in the sensing channel which reduce the charge screening at the concave regions. Moreover, the deformed graphene could exhibit a band-gap, allowing an exponential change in the source-drain current from small numbers of charges. Collectively, these phenomena allow for ultrasensitive electronic biomolecular detection in millimeter scale structures.

All-electrical detection of biomolecules and specifically nucleic acids are of great interest for gene-expression investigations¹, pharmacogenomics², drug discovery³, and molecular diagnostics⁴⁻⁶. These methods also offer considerable promise for forensics⁷, environmental monitoring⁸ and global personalized medicine⁹. In particular, field effect transistor (FET) based detection of nucleic acids has drawn great attention as label-free and highly sensitive biomolecular sensing platform which can be readily integrated with other electronic components such as data analyzers and signal transducers. 2D materials such as graphene are attractive due to their unique properties such as ambipolar field effect, high carrier mobility, biocompatibility, mechanical strength, and flexibility¹⁰. 2D materials intrinsically exhibit high sensitivity in detection of charged biomolecules due to their ultimate thinness and extremely high surface to volume ratio. Compatibility to the conventional CMOS fabrication process is another potential advantage of using 2D materials, which carbon nanotube, Si-nanowire, nanoparticles do not have. Especially, large area graphene, which is grown through chemical vapor deposition (CVD) method, has been utilized in electrical systems, such as FET device for bio-sensing⁵ including detection of pH¹¹, microorganisms¹², blood sugar¹², and more specifically, proteins at concentrations of 10 fM^{12,13}, and nucleic acids (DNA or RNA) at the 100 fM concentrations^{5,14}. There are a few reports that showed DNA and RNA detection at aM level, however, had significant level of background noise and lacked robust controls^{15,16}. Further sensitivity would be highly desirable for detection of very rare molecules such as micro RNA (miRNA) or cell-free DNA (cfDNA) from unamplified samples¹⁷.

It is important to detect DNA/RNA such as miRNA circulating in serum or plasma with high ionic strength. Such detection could enable liquid biopsy, which can replace invasive tumor-tissue biopsies in many diagnostic applications. The existing approaches to monitor cancer-related miRNA is based on the polymerase chain reaction (PCR)¹⁸. Unfortunately, PCR is susceptible to interference by the inhibitory factors in biological samples, therefore not suitable to analyze miRNA directly from blood or serum samples¹⁹. Moreover, the result can be misinterpreted by bias and artifact due to the amplification efficiency of different sequences²⁰. Therefore, there is an urgent need to develop amplification- and purification-free method to directly detect miRNA from biological samples such as serum.

One of the major hurdles to lower the detection limit of FET-based biosensor is shielding of the molecule charge by the counter ions in solution (termed Debye shielding)^{21,22}. Outside the Debye length, which is less than 1 nm in physiological solutions, the charges are electrically screened. An increase in the Debye length can result in reduced screening effect and allow for a more sensitive electrical detection of charged biomolecules. While methods have been proposed to overcome this intrinsic limitation of FET biosensors^{21,22}, these have focused on detecting biomolecules which are larger than the Debye length itself. None of the reports has tried to overcome the concentration limit of detection by modulating the Debye screening. Moreover, none of the works have modulated Debye screening in clinical solution such as serum or plasma^{21,22}.

Computational reports have also predicted that the curved morphology of sensing materials can affect the Debye length (or volume), which can increase in concave regions of a nanowire sensor²³. Thus, we hypothesized that if the surface of the sensing channel can be curved or bent at the micro- and nanometer scale, the Debye length could be modulated resulting in higher sensitivity. Previous works have shown that 3-dimensional ‘crumpled’ graphene can be created by deformations at the micro and nano-scale on pre-strained thermoplastics by relieving the stress and inducing buckle delamination of the graphene²⁴. This approach can be used to engineer curving and bending of 2D materials and thin films. Several applications have been investigated using the mechanically-tunable crumpled graphene such as stretchable photosensors²⁵, nanoplasmonic sensor²⁶ and strain gauges²⁷, and the in-plane strain can change the electronic properties of graphene, opening a band gap with a 1% stretch^{28,29}. Moreover, crested 2D materials FETs recently showed large increase in the mobility as compared to standard devices³⁰. It has also been separately shown that flat 2D semiconducting material such as MoS₂ can be 10 times more sensitive than flat semi-metallic 2D graphene for biosensing applications³¹. Hence, we hypothesize that nanoscale bending of 2D graphene in 3-dimensions could result in high sensitivity due to modulation of the Debye length (or volume), and possibly due to strain induced band-gap opening in the graphene channels. Such deformed graphene (curved or bent 2D) layers have not yet been used for biosensor applications.

Here, we report the use of these deformed and bent (crumpled) graphene FET based electrical biosensors for ultra-sensitive detection of DNA/RNA molecules down to 600 zM of limit of detection (LOD) on millimeter scale structures. To the best of our knowledge, this is the highest sensitivity reported so far using any electronic biosensor for detection of DNA. Because of the simple fabrication process, the presented approach has several benefits over structures such as nanoribbons or nanopores³². The process does not require electron beam lithography to fabricate the nano-confined devices. The realization of the bent and crumpled graphene is achieved by macroscopic manipulation of the 2D layer and the resulting ‘nano’-sized features exhibit the superior sensing performance, while allowing facile fabrication and reproducibility. We demonstrate detection of 22-mer single and double stranded molecules by adsorption and hybridization experiments, respectively. We also showed that the target miRNA (let-7b) and a DNA probe hybridization was detectable as low as 600 molecules in 50 μL of buffer and undiluted human serum directly without amplification. The performance of the sensor was further enhanced using peptide nucleic acid (PNA) probe, which showed 600 zM of LOD, ˜18 molecules in an hour of incubation time. We show via molecular dynamics simulations the formation of electrical ‘hot spots’ at the nanoscale crumpled graphene regions where the Debye length can increase and also result in a local high potential due to the charge of DNA/RNA. Furthermore, the bending of the graphene monolayer at these hot spots can result in opening of a bandgap and provide for an exponential increase in the conductivity in vicinity of the biomolecules. These effects combined can allow for a measurable current change in millimeter scale channels even with 600 zM concentration of the target molecules.

Results: Characterization of Deformed Graphene FET Biosensor

The scheme for the graphene FET DNA sensing is illustrated in FIGS. 1A-1G. Probe DNA will be anchored via a linker molecule on flat and crumpled graphene channel of FET sensors, and the target DNA will be hybridized. The concept of Debye length modulation along the flat and crumpled graphene is depicted in FIG. 1A. The dotted blue line represents the Debye length from the graphene surface. Flat graphene has a constant Debye length; however, the Debye length fluctuates at the peaks and the valleys of the crumpled graphene. The changes in Debye length on the crumpled graphene expose more of the DNA as compared to the flat graphene. Debye screening is weaker on the crumpled graphene, potentially enabling a higher sensitivity detection of DNA. The FET fabrication process is shown in FIG. 1B. The graphene channel (1×15 mm) was transferred onto a polystyrene substrate using an established method²⁴. To produce the crumpled FET device, the graphene on polystyrene substrate was annealed at 110° C. for 4 hours. This annealing process induces the shrinkage of the underlying pre-strained thermoplastic substrate which results in buckling of graphene²⁴. The optical image of the crumpled graphene device is in FIGS. 8A-8C. For the flat FET device, the annealing process was omitted. Then the source and drain metal electrodes were formed and a solution reservoir was created. When performing fluid measurements, buffer solution was placed in the reservoir in the device and a gate voltage was applied directly to the top of the buffer solution.

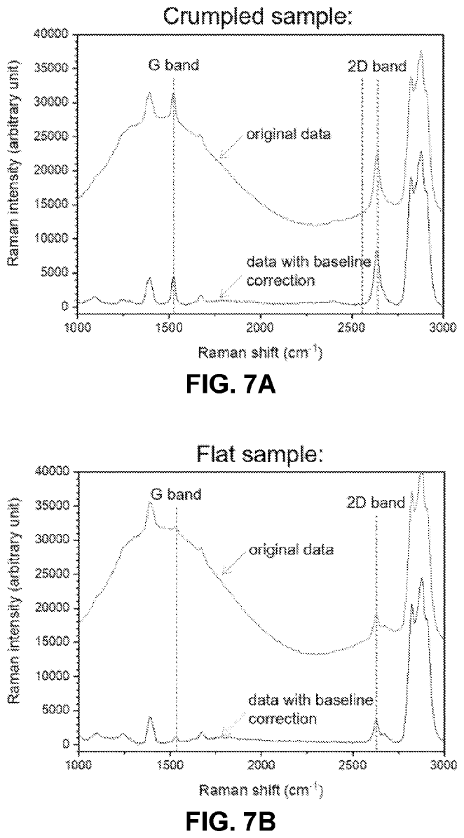

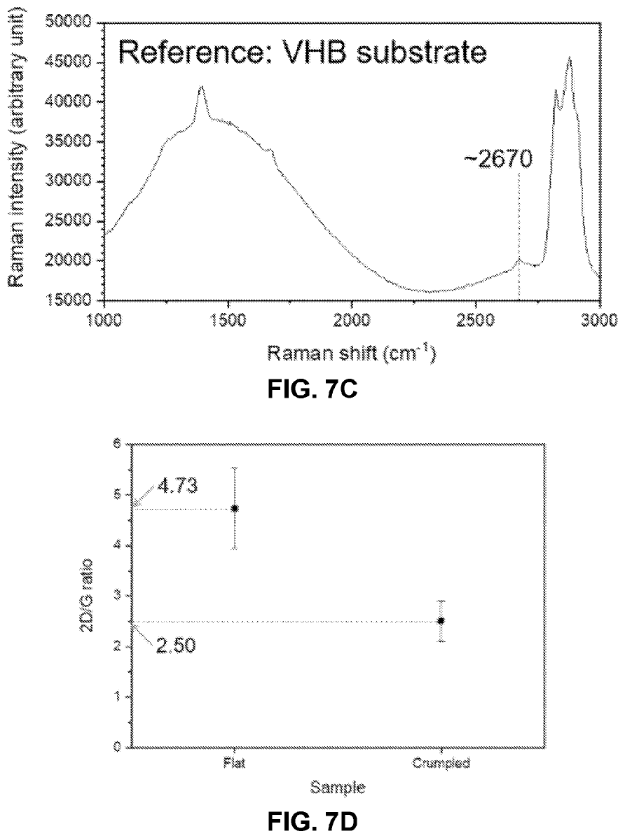

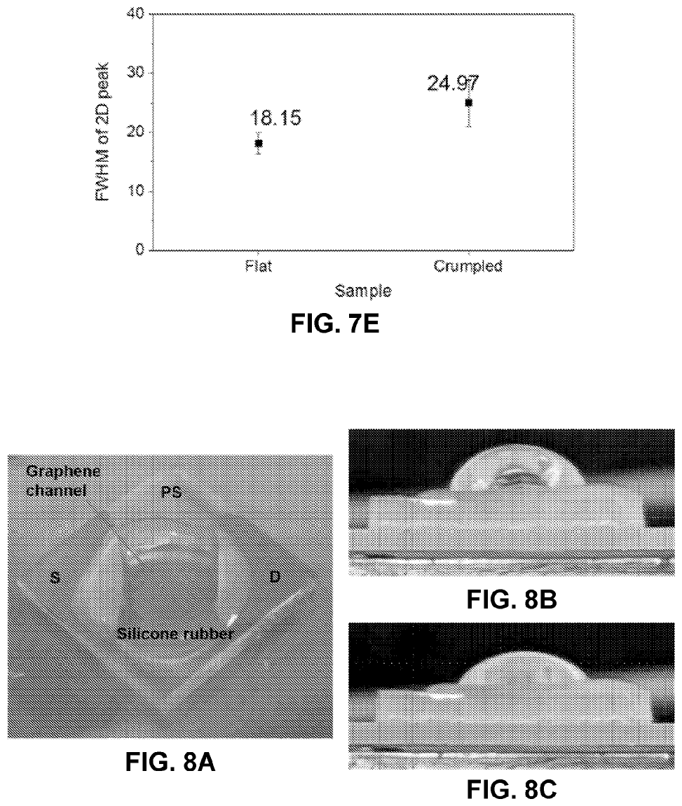

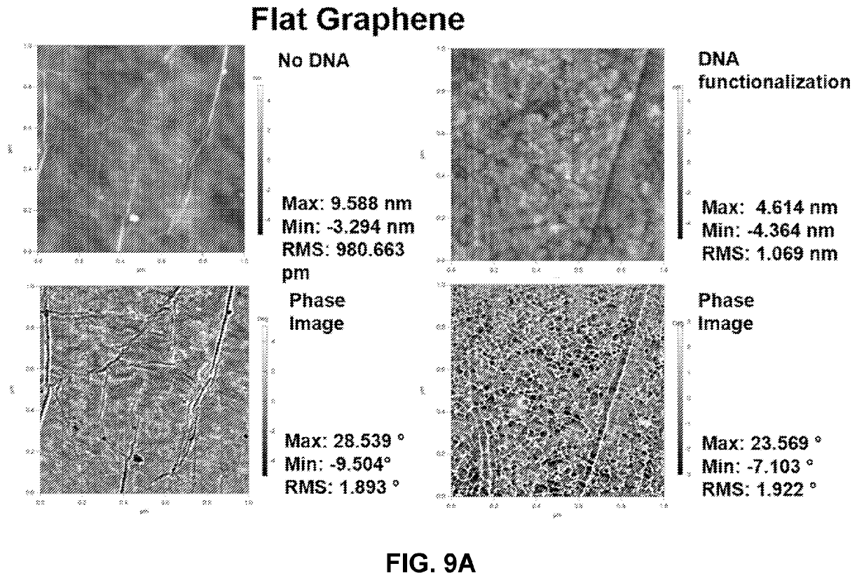

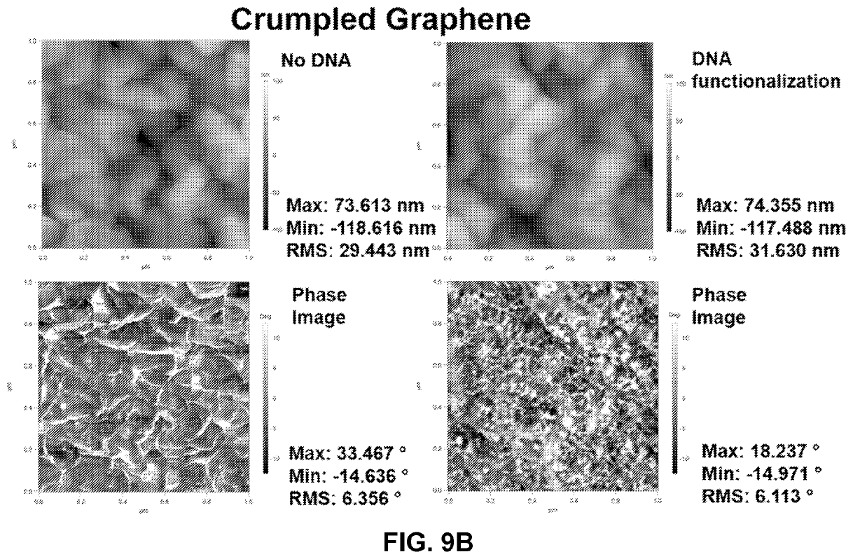

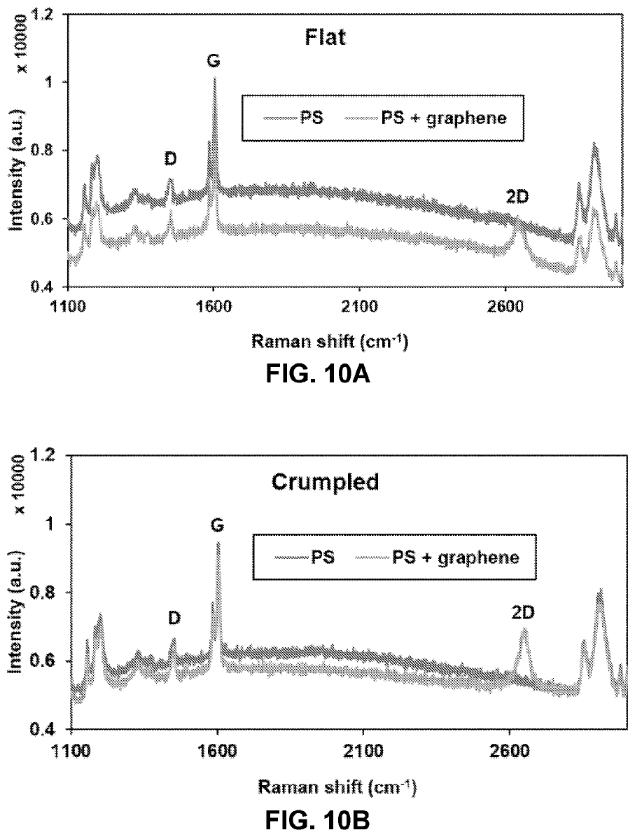

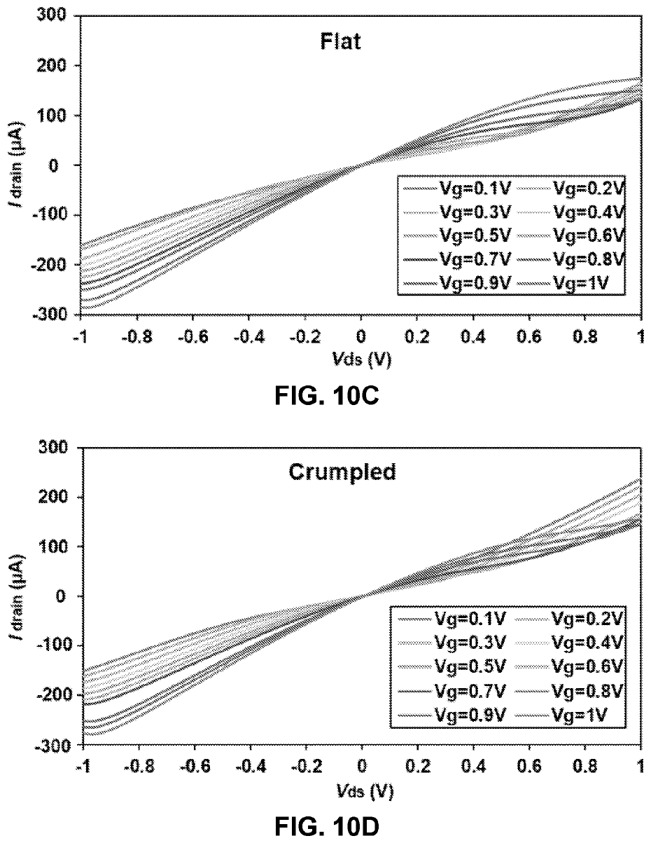

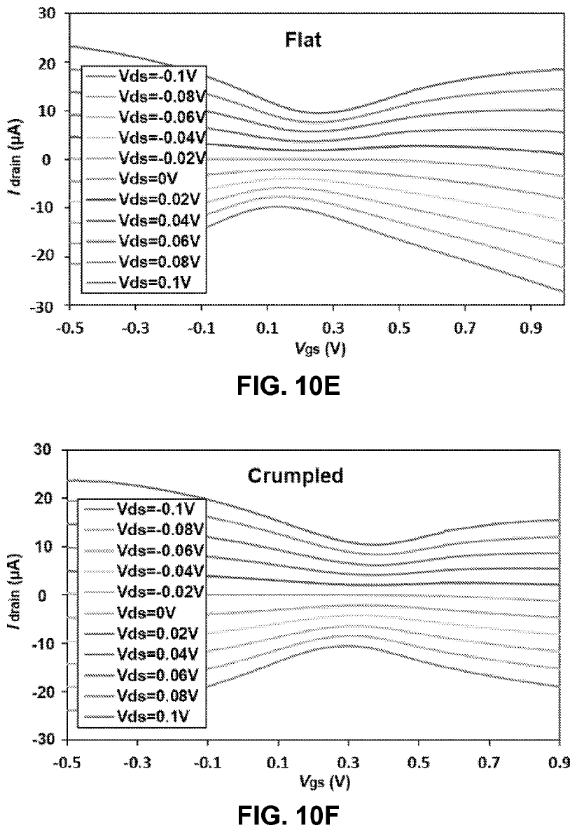

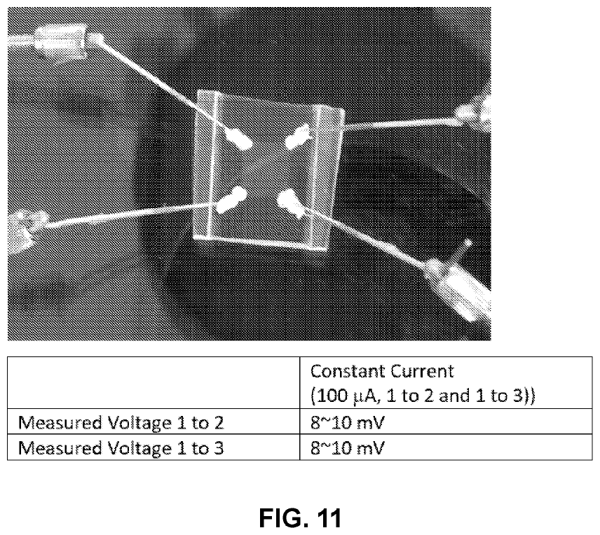

The morphology of the flat and crumpled graphene was characterized by scanning electron microscope (SEM) and atomic force microscope (AFM). Disorganized herringbone-like structures were observed (FIG. 1C) and the size of large wrinkles were a few microns, however, when further magnified, finer wrinkles as small as a few hundred nanometers were observed. Debye length modulation would be attributed to those small wrinkles²⁴. AFM derived topography data (FIG. 9B) shows increase in the surface roughness for the crumpled graphene. It should be noted that the measured topography and RMS roughness values are underestimated due to the resolution of the AFM tips being larger than the ultrafine nature of the crumples. Raman spectroscopy analysis shows that the quality of graphene was intact after the crumpling process (FIG. 1D). The Raman D-to-G peak of the flat and crumpled graphene had similar intensity ratio. The background Raman spectrum of polystyrene substrate only is also shown in FIG. 1D²⁴ and in FIGS. 10A-10B. To determine if the graphene was still monolayer after the crumpling process, VHB (3M, Very High Bond, 3M) substrate was used and analyzed (FIG. 7A-7E). To confirm that crumpled texturing will not adversely affect graphene's electrical properties, the source-drain current was measured, and the resistance was found to be between 8 to 12 kΩ over the measured devices. Sheet resistance of the graphene was measured by van der Pauw method and found to be ˜450Ω (FIG. 11)³³.

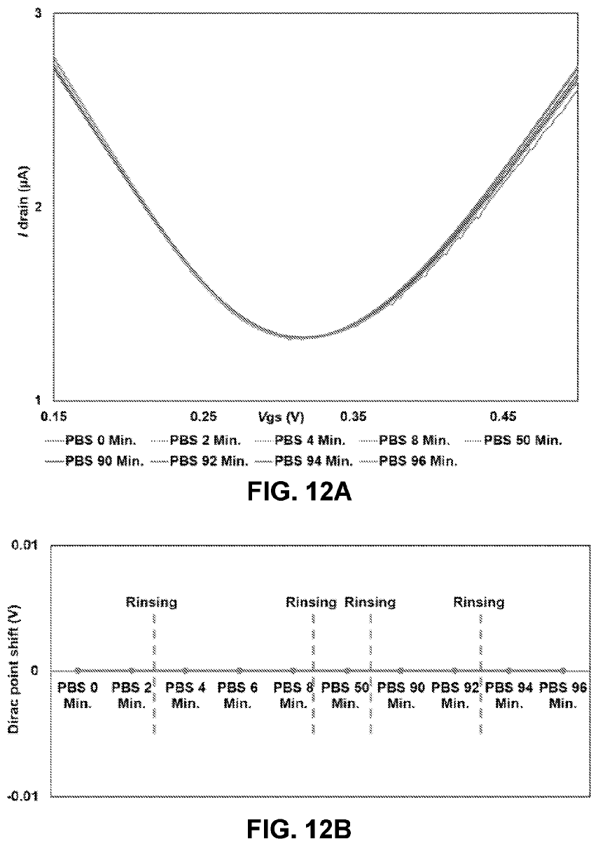

Then the flat and crumpled graphene FET sensor were characterized with liquid gate. Graphene FET generally shows intrinsic p-type behavior due to negatively charged impurities underneath the graphene sheet which are trapped during the transfer process³⁴. The conductance of the graphene channel was modulated by liquid-gate potential applied to the solution reservoir. The accumulated ions modulate the conductance of the graphene channel by either p- or n-doping effects because of the ambipolar characteristics of graphene. The ambipolar transport characteristics of the FETs are illustrated in FIG. 1E and FIGS. 10C-10F. In I_{d} vs. V_{gs} curves, the Dirac points shifted as V_{ds} changed. Dirac points were positioned between V_{gd} of 0 and 0.5 V, which is mainly related to the work function difference of Ag/AgCl gate electrode and the graphene. The measurements were repeated over time and confirmed that the Dirac voltage values were stable in PBS buffer solution before using the devices for measurements (FIGS. 12A-12B).

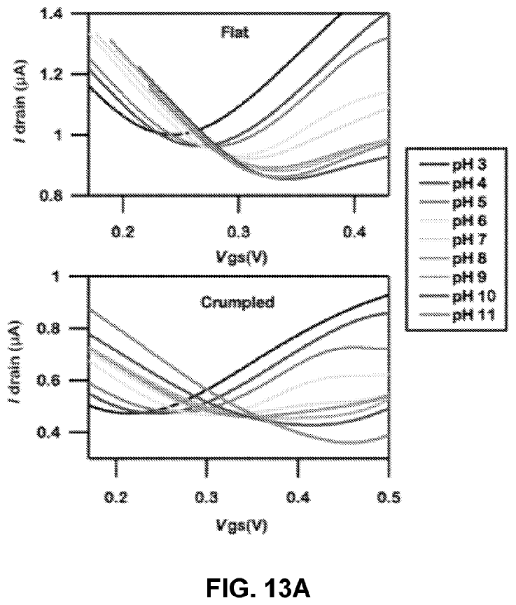

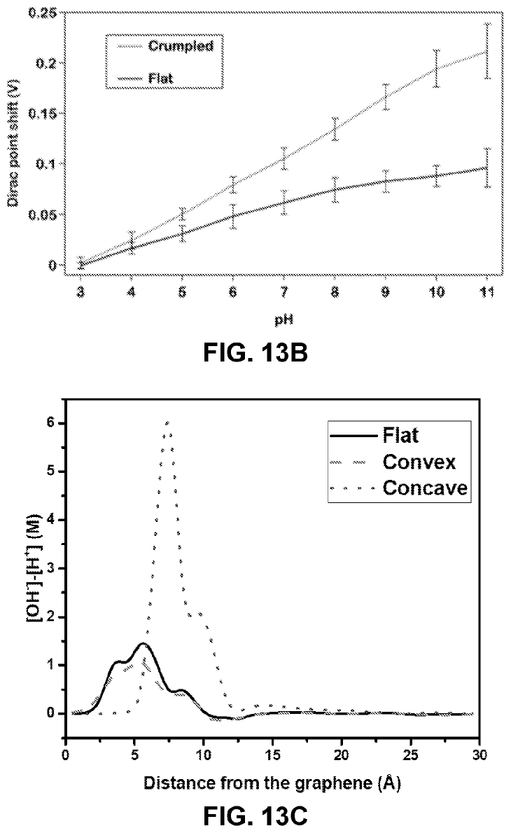

Performance comparison of crumpled and flat graphene devices: The devices were then used for pH sensing and when the H+ ion concentration changes, the current through the transistor will change accordingly (FIG. 1F). Interestingly, the crumpled graphene ISFET showed a larger shift in Dirac point from pH 3 to pH 11 as compared to the flat graphene ISFET (FIGS. 13A-13C). This might happen because the nanoscale morphology of the crumpled graphene perturbs the regular electrical double layer (EDL) and small ions could be trapped in the crumpled structure. This will be discussed later in the computational simulation section.

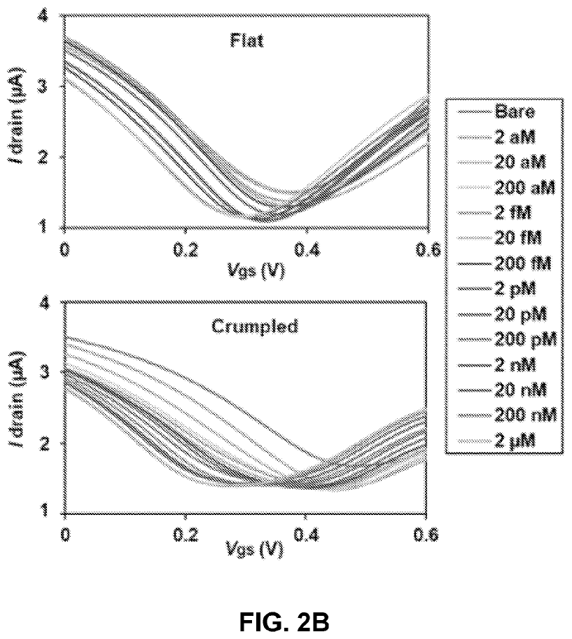

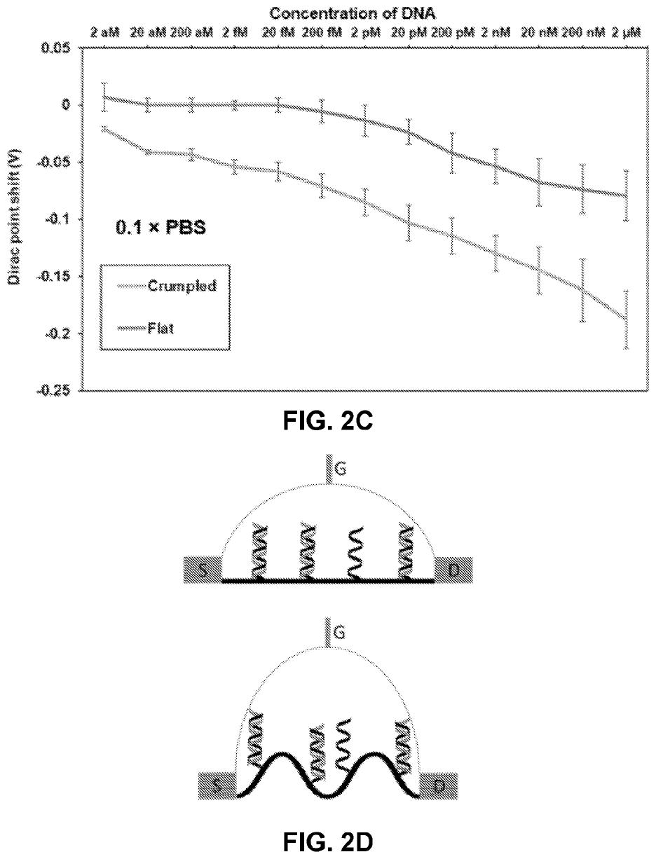

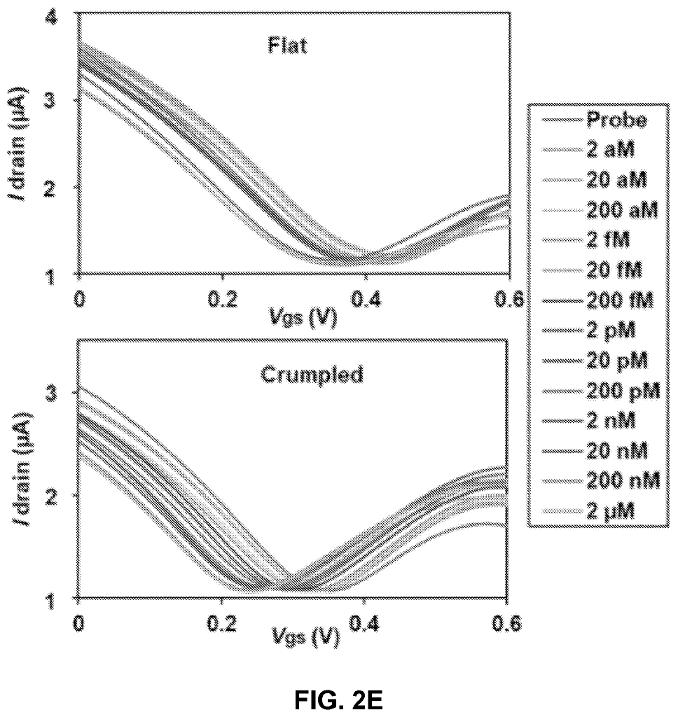

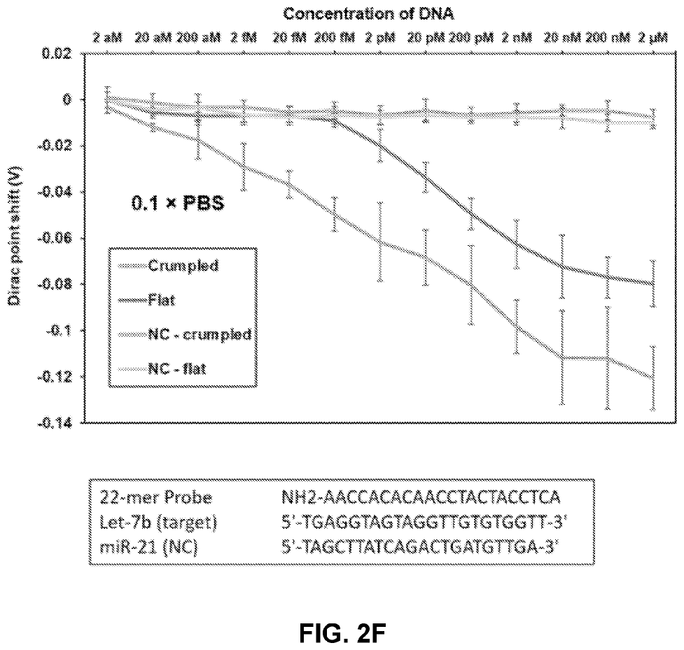

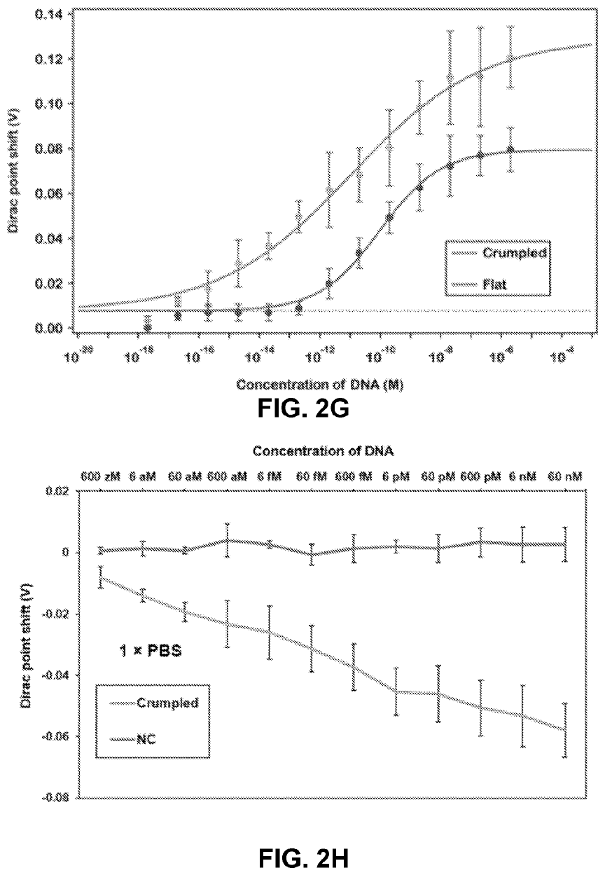

We then examined electrical sensing of DNA in fluid. Consistent with prior reports^{5,14}, we also observed that the physical contact of ssDNA strands (let-7b sequence, Table 1) imposed n-type doping effects on flat graphene, resulting in a negative shift of Dirac point as shown in FIGS. 2A-2C. This is attributed to the interaction between graphene and electron-rich nucleobases in DNA molecules³⁵. When the screening effect caused by ions in the buffer solution becomes strong, the charge impurity scattering caused by adsorbed DNA molecules is reduced³⁶. The electrical effect of DNA on graphene gets weaker, generating smaller signals. For the flat graphene FET device, no significant shifts were observed below 2 pM concentration, whereas for higher concentrations, the Dirac point shifts were clearly observed (FIGS. 2A-2C). As higher concentrated DNA molecules were introduced, the IV curves gradually shifted to the left with the overall shifts of up to 80 mV. The crumpled FET device showed much large differences in the Dirac shift and even 2 aM of DNA molecules in solution resulted in IV shift of up to 20 mV and the total shift was ˜180 mV (FIGS. 2D-2F). This corresponded to about 600 molecules in 50 μL solution added to the sensor.

TABLE 1
DNA, PNA and RNA sequences used in Example 1

| SEQ ID | | |
|---|---|---|
| No: | Description | Sequence |
| 1 | 22-mer (Probe) | NH2-AACCACACAACCTACTACCTCA-3′ (DNA) |
| 2 | | 5′-AACCACACAACCTACTACCTCA-OO (PNA) |
| 3 | Let-7b (Target) | 5′-TGAGGTAGTAGGTTGTGTGGTT-3′ (DNA) |
| 4 | | 5′-UGAGGUAGUAGGUUGUGUGGUU-3′ (RNA) |
| 5 | miR-21 (Negative | 5′-TAGCTTATCAGACTGATGTTGA-3′ (DNA) |
| 6 | Control) | 5′-UAGCUUAUCAGACUGAUGUUGA-3′ (RNA) |
| 7 | AFM imaging 1 | 5′-TGA AAG IGT TTT AAT AGA ATT TTA AAA |
| | | IAC TIG TAI A-3′ |
| 8 | AFM imaging 2 | NH2-CCT TAT TTC TAC CAG TCT TTT AAA |
| | | ATT CTA TTA AAA CCC TTT CA-3′ |
| 9 | 3 nt short target for | 5′-TGAGGTAGTAGGTTGTGTG-3 ′ |
| | charge layer distance | |
| | experiment | |

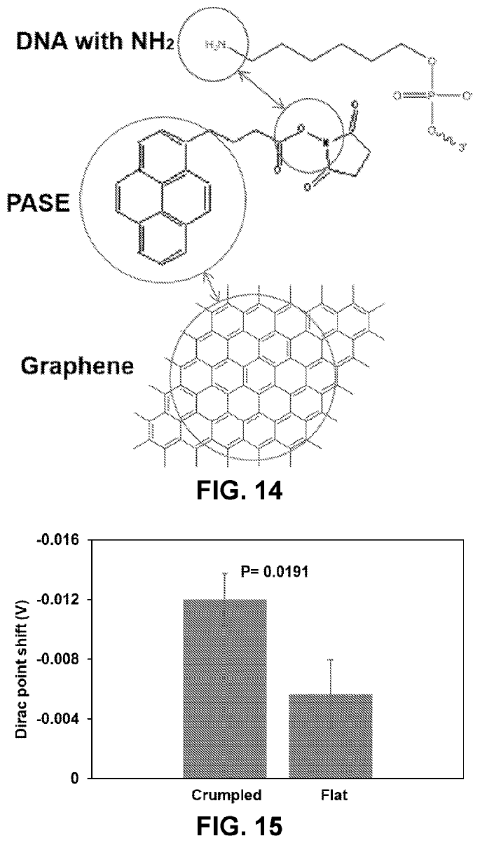

Next, we investigated DNA hybridization to measure the sensitivity of the FET biosensor. Probe DNA molecules were immobilized as reported previously (FIG. 14 and Table 1)^{5,14}. To verify that the DNA molecules were immobilized on the graphene, the surfaces of flat and crumpled graphene were probed using an AFM. The increased surface roughness was observed consistently for both flat and crumpled graphene (FIGS. 9A-9B), pointing to immobilization of the probe DNA. As shown in FIGS. 2D-2F, we were able to see that the Dirac point of the graphs shifted toward the left with increased concentration of the complementary DNA as low as 2 aM (let-7b, Table 1). For the flat graphene devices, negligible shifts were observed from 20 aM to 200 fM, and significant shifts of 2 pM or higher when the complementary DNA was introduced. The data fits well with Sips model for both crumpled and flat devices (FIG. 2G and Table 2)⁶. For the crumpled graphene device, 20 aM concentration of DNA showed a 12-mV shift. Some devices even showed a few mV of shift at 2 aM, however, these overlapped with standard deviation of negative controls. Therefore, it is reasonable to conclude that 20 aM was the limit of detection. The p-value of Dirac point shifts between crumpled and flat graphene FET biosensor at 20 aM of target DNA hybridization is shown in FIG. 15. This indicates that the crumpled graphene FET biosensor reported here exhibits the highest sensitivity reported to date (20 aM in 50 μL, ˜600 molecules) which is about 10,000 times more sensitive than prior reports from electrical biosensors³⁵.

TABLE 2
Fitting Parameters used in Sips model in this work.

| | crumpled | flat |
|---|---|---|
| A | 0.122 ± 0.007 V | 0.072 ± 0.002 V |
| a | 0.200 ± 0.021 | 0.436 ± 0 .053 |
| Kₐ | 1.12e−11 ± 9.44e−12M | 9.71e−11 ± 3.37e−11M |

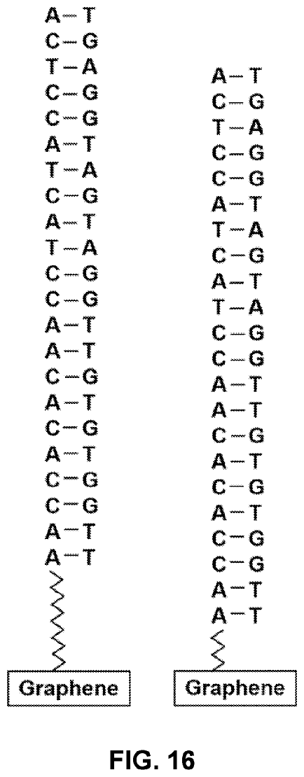

We also repeated the hybridization tests using PNA probe. It has been reported that PNA probe showed one order of higher sensitivity as PNA does not have the negative charges originated from phosphate backbone of DNA. Moreover, PNA does not need addition of NH2 functional group to react with the linker (Pyrenebutanoic acid succinimidyl ester) (FIG. 16), which reduces the distance between PNA probe and the graphene surface as compared to the DNA probe. Hence, a higher sensitivity is expected when using PNA probe³⁷. Surprisingly, the LOD was improved down to 600 zM, which is ˜18 molecules of DNA (FIG. 2H). Note that the total Dirac point shift is smaller than using DNA probe, as PNA test were measured in 1× PBS while 0.1× PBS was used for the DNA probe test.

Taking into account convection-diffusion-reaction considerations³⁸, evaporation induced convection and surface roughness effects on molecular absorption can facilitate the transport of nucleic acids to the graphene surface, reducing the diffusion-reaction time and result in high-sensitivity detection³⁹⁻⁴¹. While about 35% of initial volume was evaporated in lhr in our experimental set up (FIGS. 8B-8C, see below for detailed explanation), target molecules could be transported along convection flows to the vicinity of the sensing surface (see below). The surface roughness of crumpled graphene may also influence the molecular reaction process, compared to a flat graphene. As the crumpled graphene forms randomly oriented valleys-and-peaks, the molecular residence time can increase by 10²× as compared to the diffusion time scale^{42,43} (see below for detailed explanation).

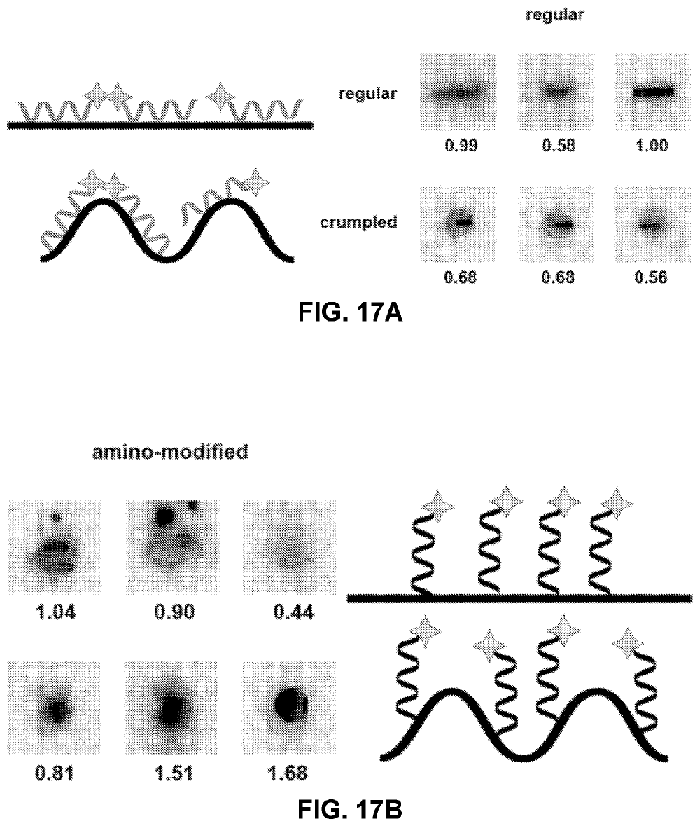

We also performed quantification of DNA attached on graphene with radioactive labeling to see if there was a difference in the density of attached DNA between the flat and the crumpled graphene surface. The relative signals from flat and crumpled graphene were similar to conclude that this high sensitivity of the crumpled graphene FET sensor was not from a difference in density of the attached molecules (FIGS. 17A-17B).

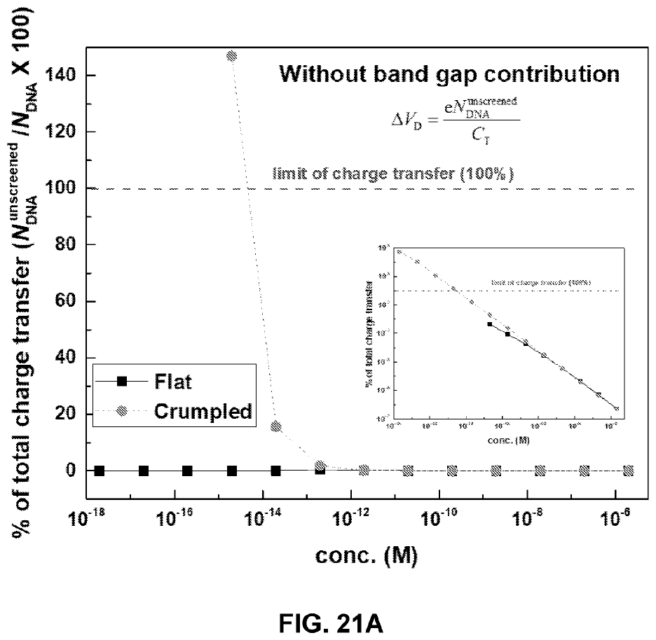

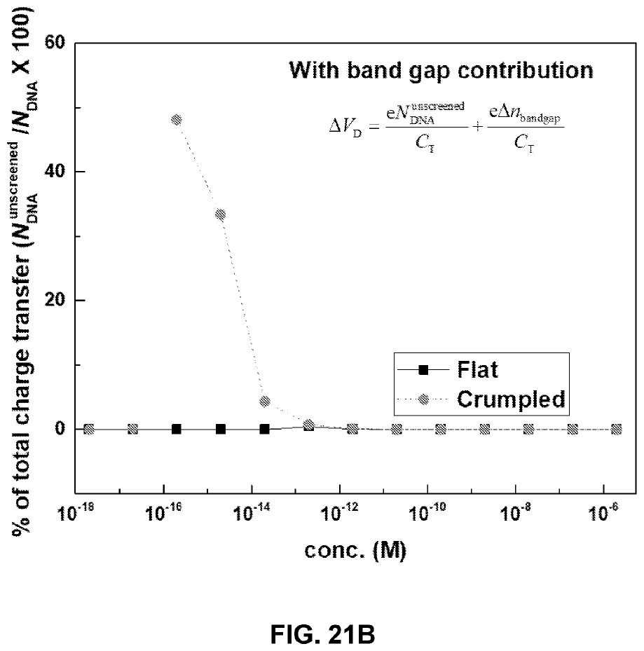

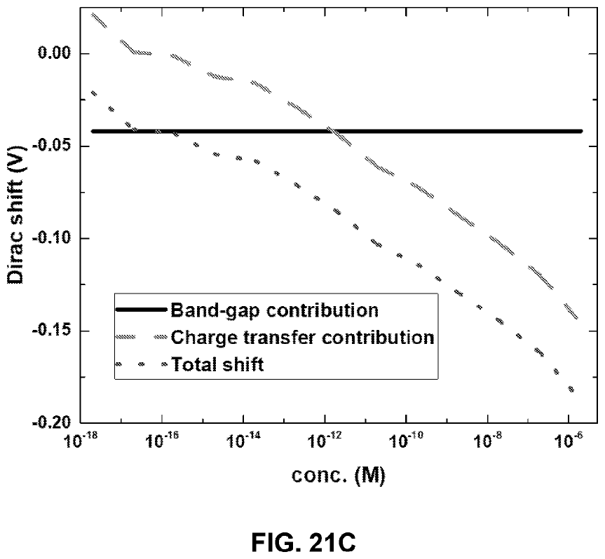

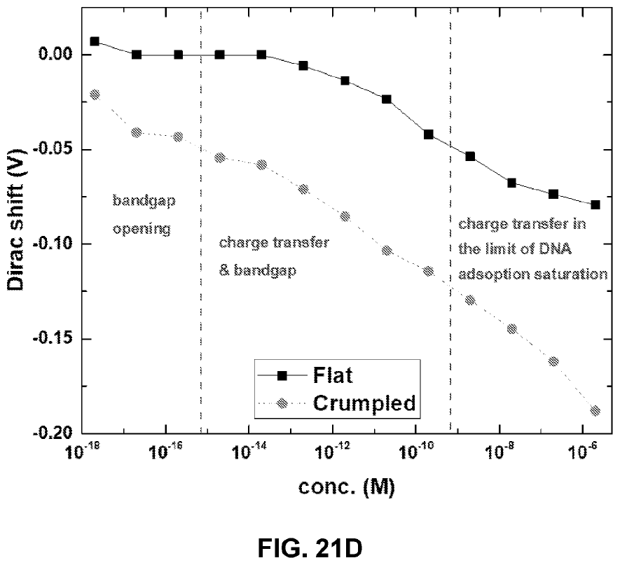

To demonstrate the capability of realistic applications of the platform, we performed miRNA detection spiked in undiluted human serum, which is not only highly ionic but also a complex mixture of biological components. In the same testing time (1 hour), we measured a clear Dirac point shift, but about half of the earlier measurements in PBS. We measured shifts at 20 aM compared to the negative control tests as shown in FIG. 21. The concentration level of let-7b in human blood is known to be in the fM range⁴⁴ and flat graphene FET sensor is not capable of detecting it at this range. Our results demonstrate that the crumpled graphene FET shows distinct signals in the aM to fM range using direct label free electrical detection of the miRNA molecules as an important application, and not require purification or extraction of the molecules as required by existing technologies.

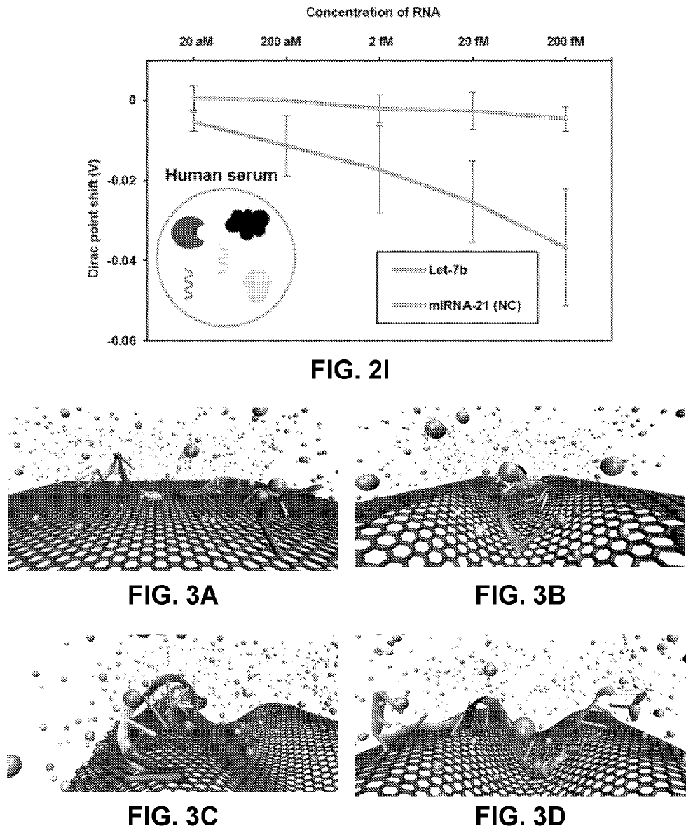

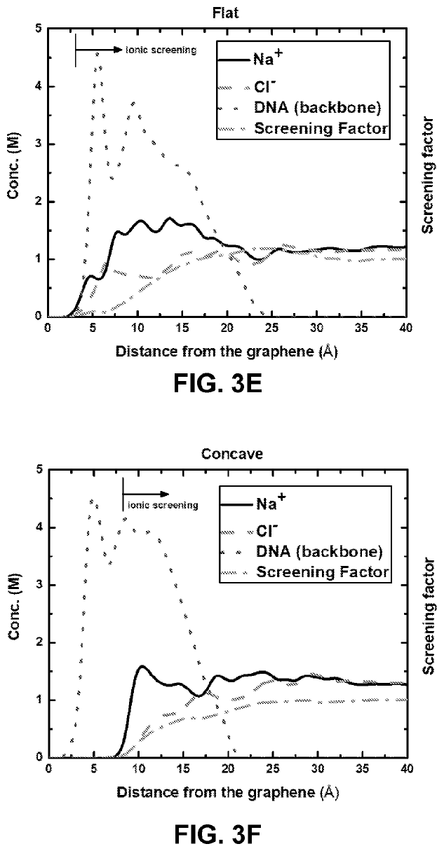

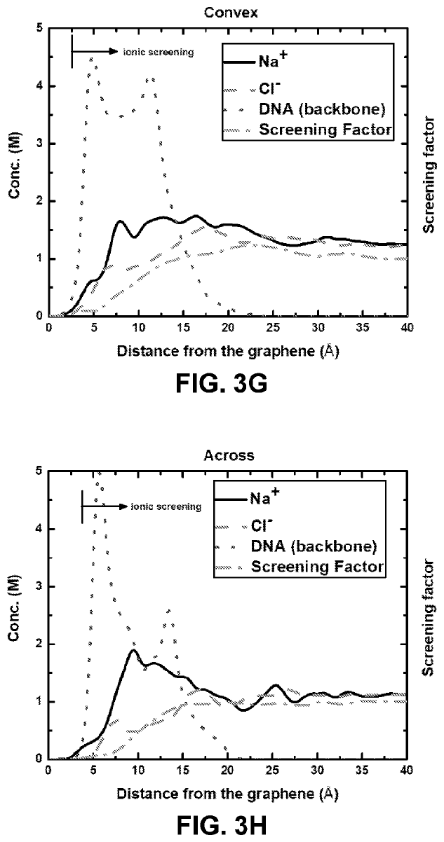

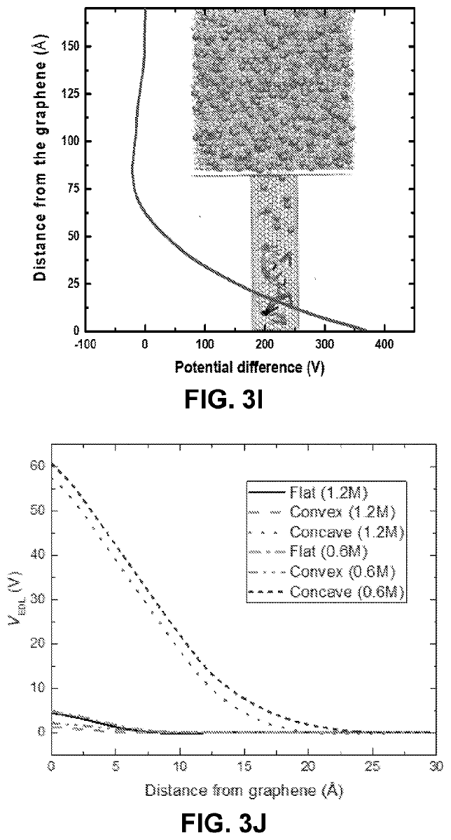

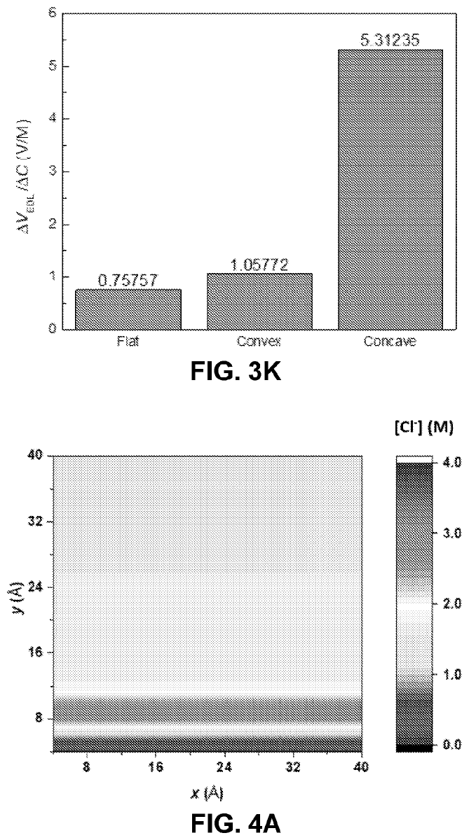

Origin of Dirac Shift: To investigate the phenomena underlying the experimental results and the effect of ionic screening of DNA molecules, we studied the electrostatics and charge distribution of DNA and ions near flat and deformed graphene surfaces using molecular dynamics (MD) simulations (see the methods section for simulation details). The presence of the unscreened charges (acting as dopants) carried by DNA molecules near the surface of the graphene produces long range electrostatic potential leading to a change in the charge carrier density (Δn) of graphene and therefore a shift in Dirac point (ΔV_{D}) given by⁴⁵

$$Δ {V}_{D} = \frac{e Δ n}{{C}_{T}}$$

where C_{T} is the total gate capacitance, and Δn is directly proportional to the charge density of the unscreened DNA molecules (N_{DNA}^{unscreend}) adsorbed on the graphene surface⁴⁵. The counter-ion screening of the DNA molecules lowers the net charge of adsorbed DNA and affects the detection sensitivity. As shown in FIGS. 3A-3D, four different configurations are considered. In the first simulation labelled as ‘flat’, a single-stranded DNA was equilibrated on an ideally flat graphene surface. In the three other configurations, the single-stranded DNA is adsorbed to the surface along the ‘concave’ and ‘convex’ regions of the crumpled graphene, and ‘across’ the deformed graphene, respectively.

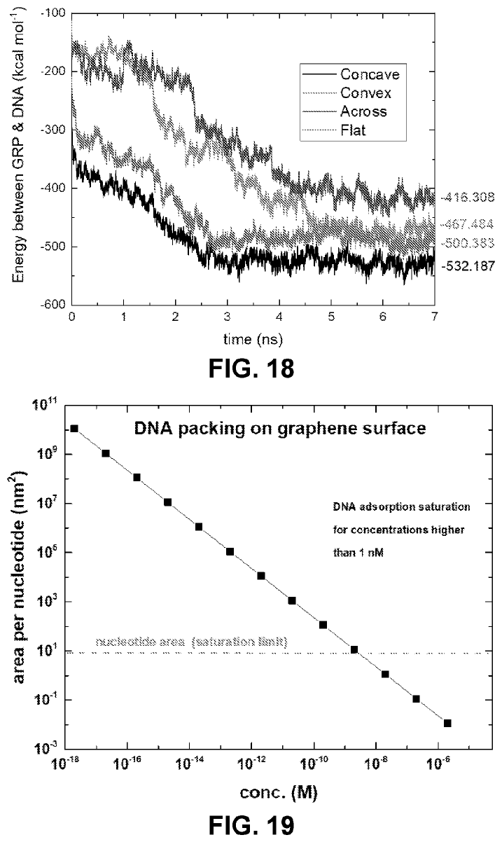

The interaction energies show that the adsorption of DNA to graphene in the concave region is the strongest. The calculated energies for the concave, convex, and across cases are −532.187 kcal mol⁻¹, −467.484 kcal mol⁻¹, and −416.308 kcal mol⁻¹, respectively (FIG. 18). We should note that due to the complexity of actual graphene structure in experiments, it is difficult in MD simulations to calculate the exact degree of adsorption based on the simulations for a single type of crumpling (the graphene surfaces considered in the MID simulations). Therefore, we only investigated the relative degree of adsorption on different graphene surfaces for a single-stranded DNA molecule. See FIG. 19 for more detail about DNA adsorption onto graphene surface.

The detection sensitivity of DNA molecules is defined by the degree to which the DNA molecules are screened by ions present in the solution²³. When the DNA molecules are isolated from the surface due to the ions present in the solution, the detection sensitivity can be significantly lower. We simulated the structure of ions and DNA relative to each other at the graphene interface for the four different configurations (FIGS. 3A-3D). The concentrations of ions (sodium and chloride) and the backbone of the DNA strand as well as the screening factor of ions are shown in FIGS. 3E-3H for the four configurations. The screening factor is then computed using the expression, where

$$S F ( z ) = {\int }_{0}^{z} F ( [ {Na}^{+} ] - [ {Cl}^{-} ] ) dz / \vert σ \vert$$

F is the Faraday constant, z is the normal distance from graphene surface (z=0 on graphene) and σ is the graphene surface charge density. As shown in FIGS. 3A-3H, because of the confined nature of the concave region, ions are excluded and are farther away from the concave graphene surface leading to increased exposure of the DNA to the graphene surface. Here, the relative position of DNA charges with respect to the ions matters. In other words, the screening by ions starts at a larger distance away from the graphene surface in the concave case leaving much of DNA charges next to the surface unscreened. This results in weaker ionic screening of DNA molecules (or higher N_{DNA}^{unscreend}) adsorbed in the concave regions. Higher N_{DNA}^{unscreend} induces more change in graphene carrier charge density (n) leading to a larger Dirac point shift. In the flat, convex and across configurations, because of weaker confinement, mobile ions are less restricted and freely present in the vicinity of the DNA molecule, screening its charge to a larger extent (lower N_{DNA}^{unscreend}).

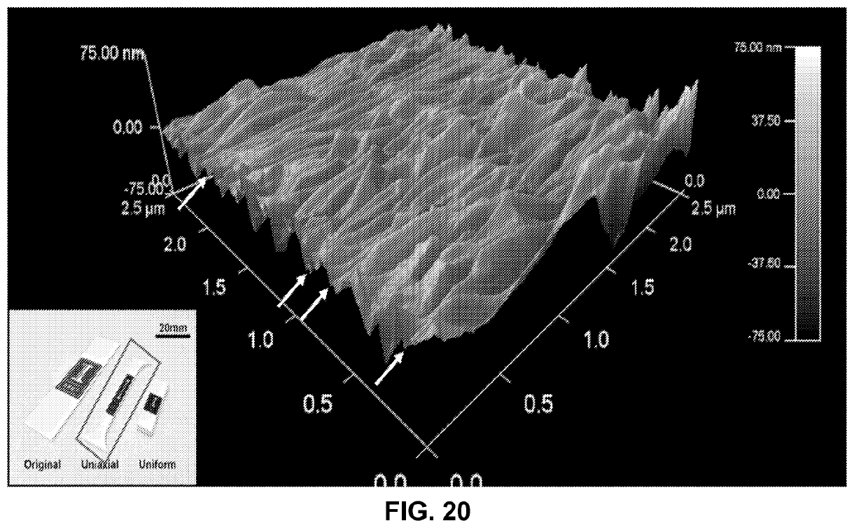

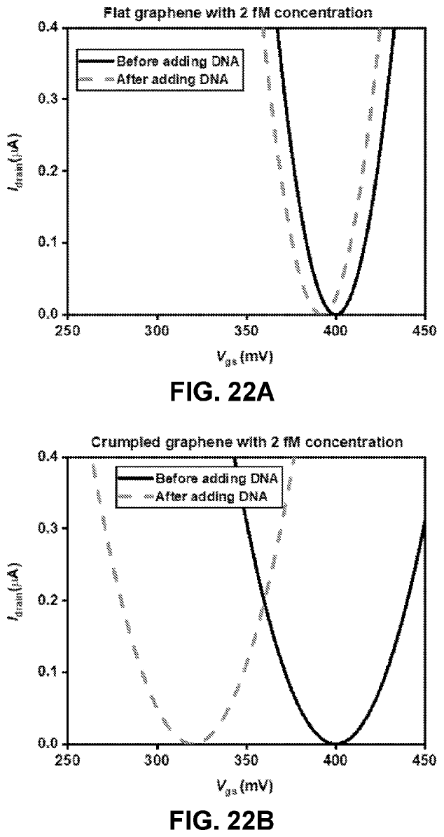

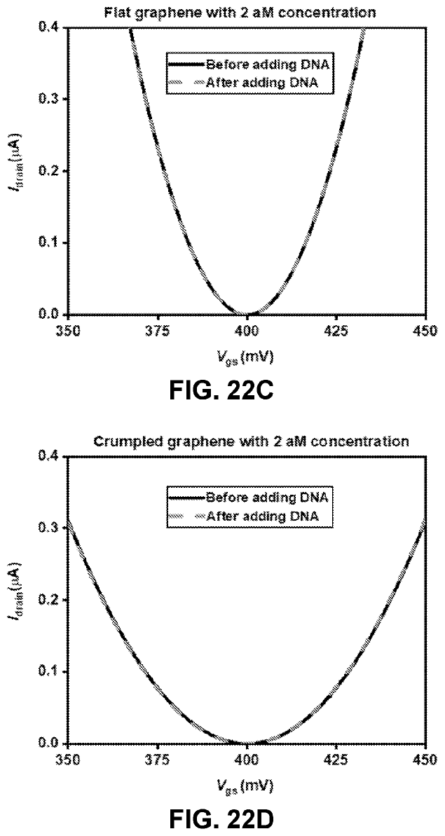

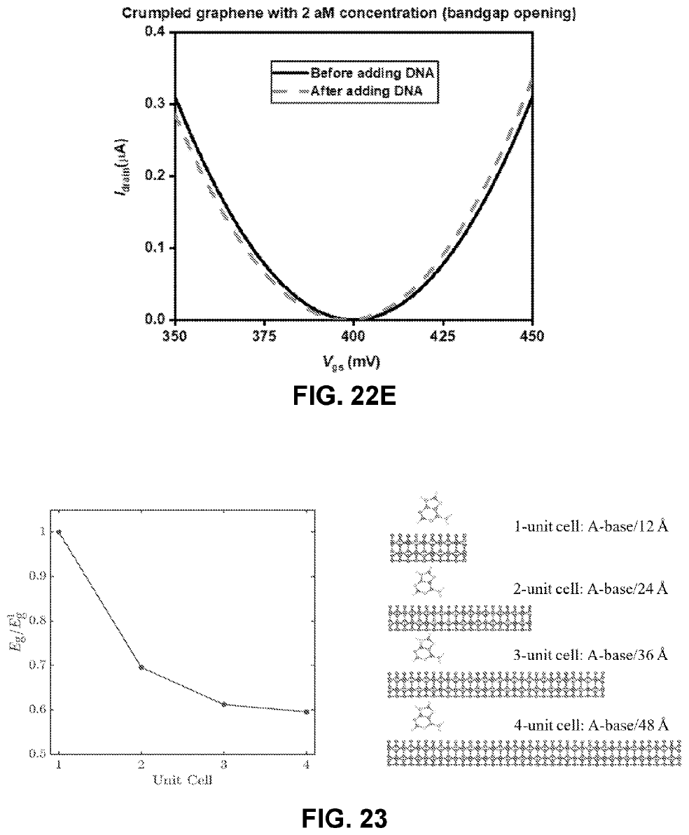

The bending used in FIGS. 3A-3D has a wavelength of 5.41 nm and amplitude of 0.73 nm. However, a variety of crumple sizes might exist in the experiments²⁵. We also modelled a narrow trench (resembling a large amplitude and short wavelength) in MD with a diameter of 2.45 nm and a length of 8nm as shown in FIG. 3I. It should be noted that the few nanometer-sized crumples are not clearly visible in SEM and AFM because of irregular feature of biaxial (uniform) crumpling process. It was reported that uniaxial crumpling is possible using the same materials and method with biaxial crumpling²⁴. As a proof of principle, we created uniaxial crumple and probed the surfaces by AFM. The image in FIG. 20 shows 10 nm or smaller sized crumples. The DNA molecule sticks to the bottom of the trench and excludes the ions from that trench. The accumulation of unscreened DNA charges results in a giant local electrical potential difference between the bottom of the trench and the solution, which modifies the charge carrier density (n) of the graphene. Such a deep and narrow trench, referred to as an ‘electrical hot-spot’, could provide very low ionic screening for an adsorbed DNA molecule by exposing most of DNA to the graphene surface. Therefore, N_{DNA}^{unscreend} is high for such areas of the graphene and hence leads to a much higher Dirac point shift. For fM and higher concentrations of DNA, the higher Dirac point shifts for crumpled graphene observed in experiments (FIGS. 2A-2I) can be explained by the fact that ionic screening is weaker and N_{DNA}^{unscreend} is larger (see FIGS. 21A-21D and 22A-22E for details).

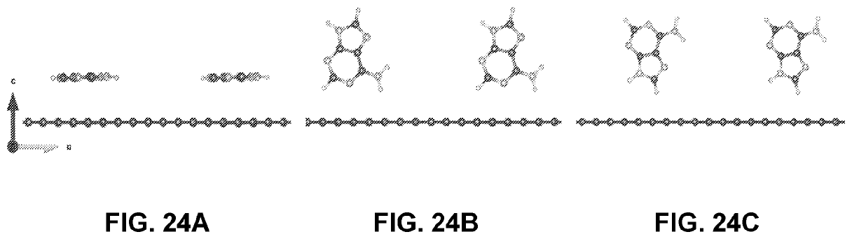

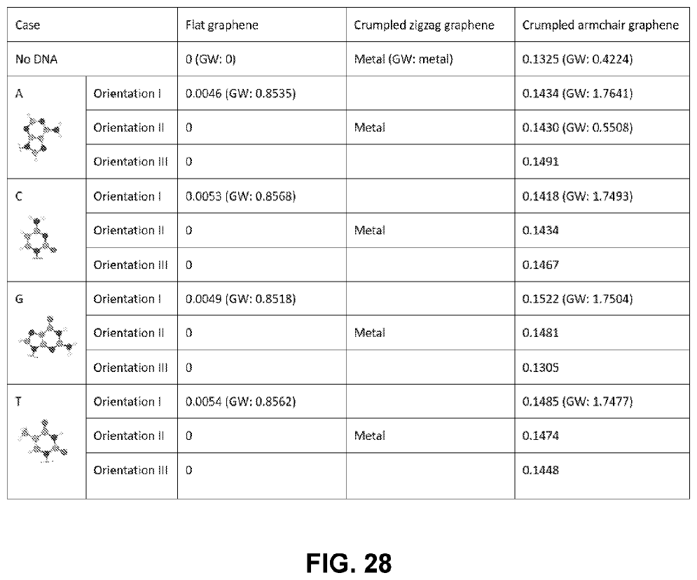

However, the observed Dirac point shift for aM concentrations on crumpled graphene cannot be explained merely by the low ionic screening of adsorbed DNA charges (assuming the maximum limit for N_{DNA}^{unscreend}). Therefore, there must be other factors that change the charge carrier density of graphene (n) to explain our measured detection of aM concentrations of DNA. Sarkar et al. showed that the existence of a bandgap in a single-layer MoS₂ leads to a higher sensitivity of charge detection compared to its graphene counterpart³¹. Here, we hypothesize the creation of a band-gap in bent graphene and next calculated the bandgap (E_{g}) for flat and crumped graphene in the absence and presence of DNA bases using density functional theory (DFT) and GW methods. Typically, GW methods are more accurate for graphene than DFT. FIG. 28 shows the computed data (see the method section and FIGS. 24A-24C, 25A-25C, 26A-26D and 27A-27B for more details). Upon addition of DNA bases, there is almost no significant bandgap opening for the case of flat graphene. However, when the graphene is deformed and crumpled along the armchair direction, the bandgap opens up to 1.7641 eV by adding DNA bases. Wang et al. showed that electronic mobility (μ) in graphene decreases with increasing bandgap

$${{(}^{μ ∝ {E}_{g}^{- \frac{3}{2}}} )}^{47} .$$

As n is inversely related to μ, the change in n (and the corresponding Dirac point shift) due to bandgap widening upon addition of DNA can be obtained⁴⁸. To achieve the observed Dirac point shift for aM concentrations used in our experiments, the change in bandgap from 0.4224 eV (crumpled graphene with no DNA) to 1.7641 eV (crumpled graphene with base A) must occur in at least ˜10⁻⁷% of the area of our graphene sensor (see the FIGS. 21A-21D, 22A-22E and 23). The combined observations and calculations explaining the change in carrier density due to charge exclusion and charge screening, coupled with opening of the band-gap, can collectively explain our experimentally demonstrated sensitivity of detection due to very few molecules in these millimeter scale FET sensors.

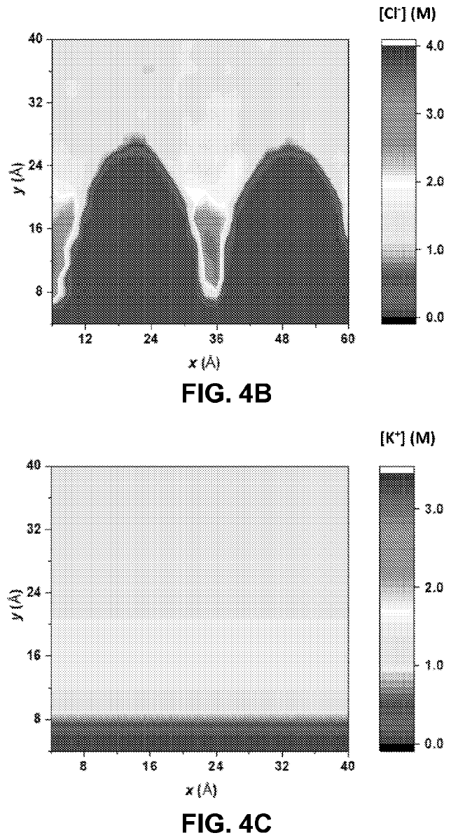

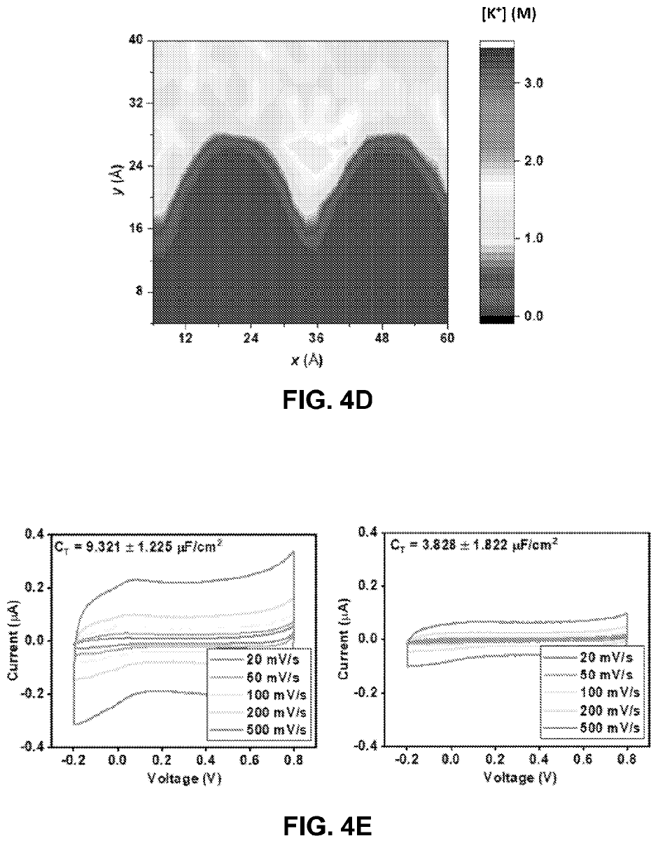

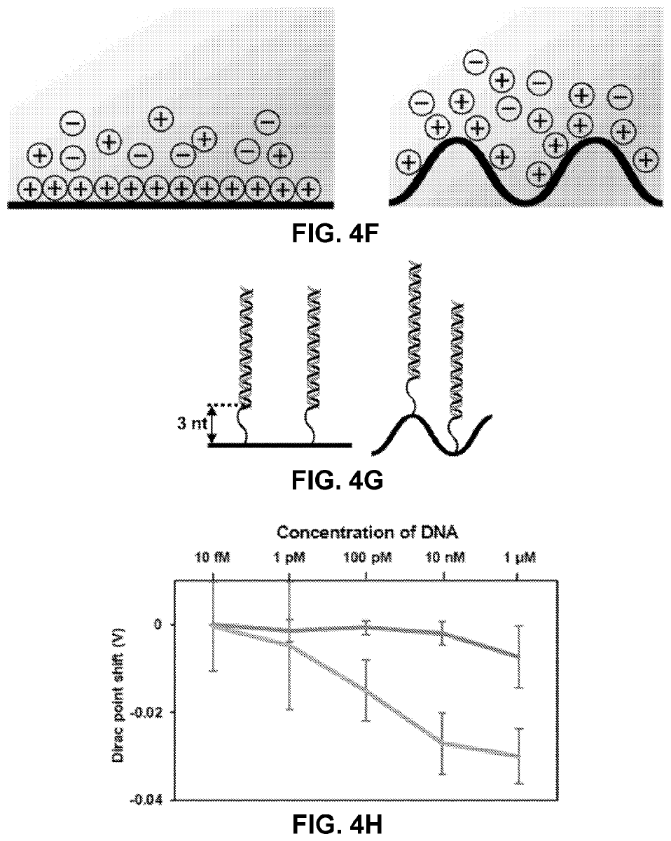

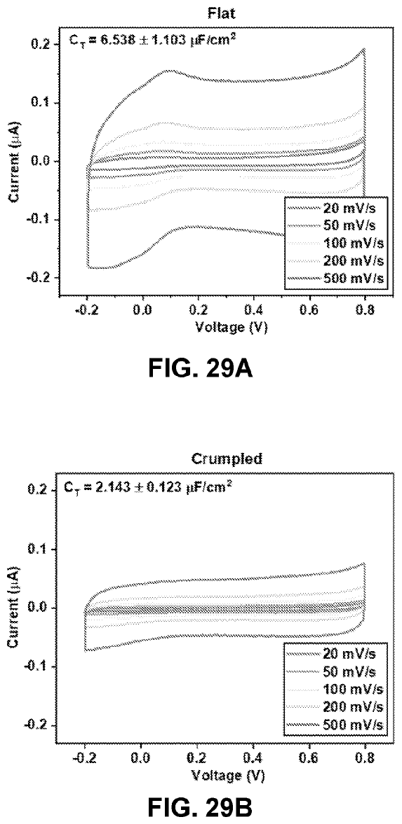

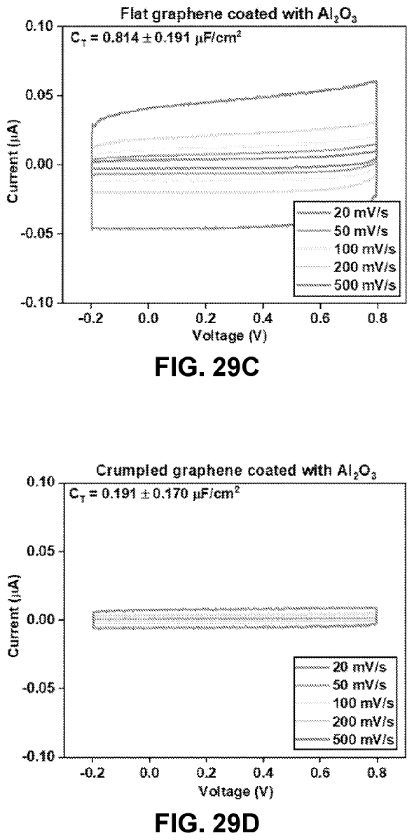

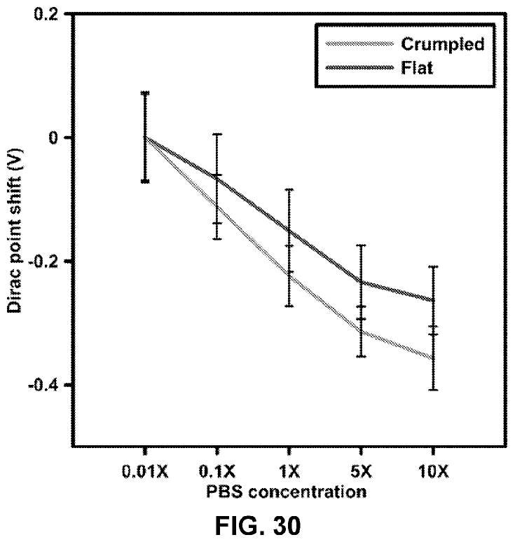

Effect of nanoscale deformation on EDL structure: To further investigate the validity of the Debye length modulation, we measured the capacitance between the graphene and the liquid electrolyte²³. The decreased screening for the crumpled graphene is seen once again (FIGS. 4A-4H), this time in the form of decreased capacitance indicating an increased screening distance, a decreased active area, or a decreased dielectric constant. A decreased active area could be less of a factor as the total currents measured are similar across the flat and crumpled structures even in the dry state. The dielectric constant itself is not expected to change much at the crumpled graphene interface^{49,50}. Hence, most of the capacitance change can be qualitatively attributed to the increased screening length. Additional sets of EDL capacitance measurement are shown in FIGS. 29A-29D. Also, different ion concentrations in the buffer solution would affect the thickness of the EDL thus resulting in a difference in the capacitance. This can also contribute to the Dirac point shift³⁵. Electrical measurements were repeated with four different concentrations of PBS without the DNA molecules and the crumpled graphene showed larger shift than the flat graphene as seen in FIG. 30. We further study the EDL length modulation in crumpled graphene in MD simulations. The molar concentration map of ions (sodium and chloride) is plotted for flat and crumpled positively charged graphene sheets in FIGS. 4A-4H. No DNA molecules are present. Compared to the flat graphene, the counter-ions in the concave region of the crumpled graphene are distributed over a longer distance away from the surface of graphene. This is consistent with the decreased screening for the crumpled graphene in the presence of DNA molecules shown in FIGS. 3A-3K.

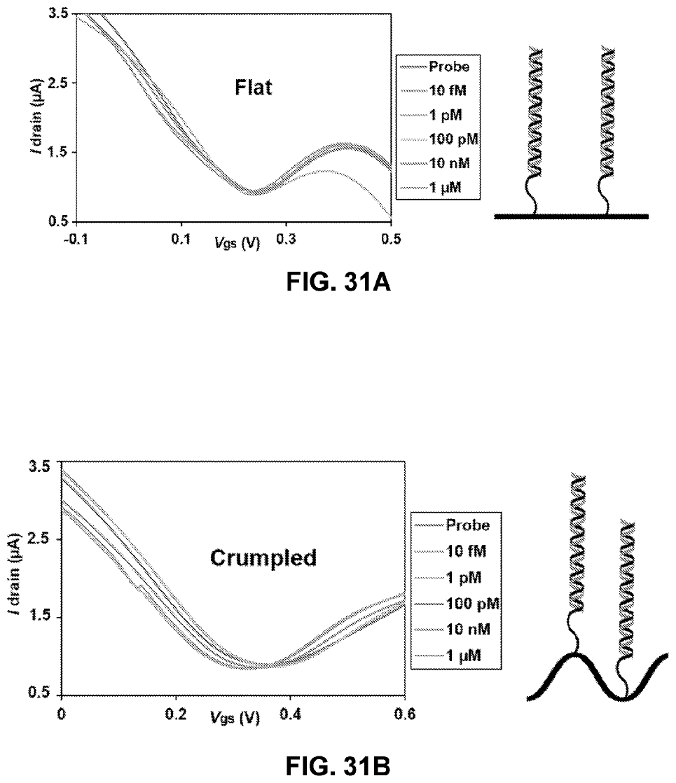

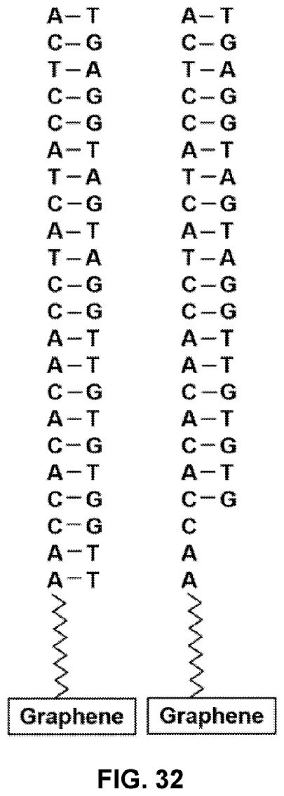

To determine if the crumpled graphene device is capable of detecting biomolecules outside the normal Debye length at a certain buffer ionic concentration, we also varied the distance of the double strand (probe+target) DNA from the surface by a distance of 3 nt⁵¹ (FIGS. 31A-31B and 32). In 1× PBS buffer solution, the Debye length is ˜1 nm, which is also about 3 nt long. As shown in FIG. 411, the flat graphene device is not able to measure the 19 nt (3 nt short) target DNA, however, the crumpled graphene clearly showed left shift of IV curves (FIGS. 29A-29D). The maximum shift was 40 mV and about 40% of the signal that fully complementary strand generated. From these capacitance measurements and the distance variation experiments, it is also reasonable to conclude that the EDL length increased by deforming and crumpling the graphene into the nanoscale morphology.

The devices can be miniaturized to micro- or nano-sized sensor in an array format. There are many fabrication and integration process challenges associated with the miniaturization. Some of these include; (i) maintaining a high quality crumpled surface if lithography is to be performed after shrinking, (ii) performing lithography first to form smaller flat graphene islands and then performing the heating or local shrinkage to cause crumpling while keep those smaller islands attached to the underlying surface, (iii) integration of silicon FETs at each pixel for row and column addressing to form larger arrays in a silicon substrate, etc.

We have demonstrated nucleic acid molecule detection on crumpled graphene FET electrical biosensor with unprecedented sensitivity using DNA and PNA as a probe. DNA adsorption and hybridization tests were demonstrated using cancer-related biomarker miRNA let-7b sequence in buffer and in human serum. The limit of detection was found to be 600 zM for crumpled graphene FET biosensor and 2 pM for flat graphene. We show via experiments and simulations that the nanoscale bending and deformations increases the Debye length in ionic solution to decrease the screening of the DNA/RNA molecules, thus contributing to the dramatic enhancement of sensitivity as compared to flat graphene FET sensors. To explain the results, Molecular Dynamics simulations revealed generation of large electrical potential due to DNA molecules in the nanoscale crevices and deformed regions that exclude ions, compounded by the formation of electrical band gap in the deformed graphene regions. These attributes coupled with increased molecular residence time due to increased roughness can result in ‘electrical hotspots’ allowing for a change in local conductivity, hence allowing atto-molar detection of DNA in millimeter scale sensors. We demonstrate the applicability of the technology by target molecules detection in undiluted human serum for cancer related miRNA. This technology can open opportunities for the development of more reliable and efficient diagnostic tools, and electrical point-of-care and implantable biosensors for early detection of biomolecules for various human diseases.

Methods:

Graphene synthesis: Monolayer graphene was grown on a copper foil via chemical vapor deposition (CVD). Before placing into the CVD furnace, copper foil was degreased with acetone and IPA, followed by nitrogen blow drying. The foil was then annealed at elevated temperature for 3 hours, while 50 sccm of hydrogen (H₂) gas flow continuously. Monolayer graphene growth was initiated when 50 sccm hydrogen and 100 sccm methane (CH₄) were introduced to the CVD chamber at annealing temperature of 1030° C., and the synthesis came to its end when two reaction gases were turned off. CVD furnace was then cooled down with flow of argon (Ar) gas, which completes the monolayer graphene synthesis process. To compare the performance among different source of graphene, graphene was also purchased from Grolltex and Graphenesquare. All the graphene showed the same LOD.

Fabrication of Graphene FET: After graphene was synthesized, the graphene/Cu foil was spin-coated with Poly(methyl methacrylate) (PMMA). Undesired graphene formed at the back side of copper foil was removed by oxygen plasma etching. The sample was cut into 1 mm×15 mm pieces with scissors or a razor blade. PMMA serves as a supporting layer while copper foil was etched by floating on 0.15 M sodium persulfate for about 5 h. The PMMA/graphene was rinsed by moving from the sodium persulfate solution to deionized (DI) water. The PMMA/graphene was then transferred onto a polystyrene substrate. The PMMA layer was removed by soaking in acetic acid for 5 min. The sample was annealed at 110° C. for 2 h to shrink the polystyrene substrate into ¼ of the original size and crumple the graphene. To fabricate transistor, conducting silver paste was used as source and drain electrodes at both ends of the graphene. Silicone rubber (DOWSIL™ 3140 RTV Coating) was used to insulate source and drain electrodes from liquid and construct a solution reservoir.

Immobilization of Probe: Pyrenebutanoic acid succinimidyl ester (PASE) (20 mM) in dimethyl sulfoxide (DMSO) was treated on the graphene overnight and rinsed with pure DMSO, ethanol and DI water; 50 μM of probe DNA solution was added on PASE-modified graphene for 3 hours. The graphene FET with probe DNA functionalization was rinsed with 1× PBS. 200 mM ethanolamine solution was treated for 1 hour to saturate the possibly unreacted amino group on PASE and rinsed with ethanol and 1× PBS solution. The volume of all treated chemicals and samples was 50 μL.

Target DNA or RNA incubation: The target DNA or RNA incubation was conducted by dropping complementary and negative control strands with concentrations that are indicated in the legends in FIGS. 2A-2I, and incubated for 1 h in the reservoir on the graphene FET chip. Then, the chip was rinsed gently with 1× PBS. For the human serum test, the chip was incubated in serum and rinsed with serum. The serum was prepared by micro-filtering human plasma. All the volume of treated samples was 50 μL.

Electrical Measurements: I-V curves and resistance were measured in a semiconductor parameter analyzer equipped with a probe station. Ag/AgCl electrode was used to apply gate voltage (V_{gs}) to the 0.1× and 1× PBS buffer solution. For the human serum test, the tests were performed in in serum. In case of DNA absorption, the graphene chips were incubated in PBS overnight because of wettability issue of hydrophobic graphene. For the serum test, the graphene chips were incubated in serum overnight and the blank measurements were repeated till there was no shift only with serum. Then target and NC RNA in serum was treated on the chip. Vg was swept from −0.5 to 1 V and drain—source voltage (V_{ds}) was picked between 0.03 and 0.1 V. Drain-source current (I_{ds}) was measured at an assigned V_{ds}.

Capacitance measurements: The capacitance measurements of flat and crumpled graphene on PS substrate were carried out using a CS 350 potentiostat (Contest, China) with three electrodes, including reference, counter, and working electrode. Here, silver chloride (Ag/AgCl), platinum (Pt), and the surface of the graphene channel on PS substrate were used for reference, counter, and working electrode, respectively. Cyclic voltammetry (CV) was chosen for the characterization method, while all three electrodes were immersed in 1× PBS solution for the measurement. Normalized total capacitance by measured graphene area (C_{T}) based on CV is shown in FIG. 4E.

MD Simulations Methods: Molecular dynamics simulations were performed using the LAMMPS package⁵². The systems were generated by the visual molecular dynamics (VMD)⁵³. To study the effect of the crumples on the EDL formation near graphene, different simulations with different graphene surface topologies were created (FIGS. 3A-3D). Flat and crumpled graphene sheets were used as two different surface topologies. The crumple has a wavelength of 5.41 nm and amplitude of 0.73 nm. Each simulation box consists of a single-layer graphene sheet, a single-stranded DNA, water and ions. The DNA has 22 bases with the sequence of AACCACACAACCTACTACCTCA (SEQ ID NO:1). The flat and crumpled graphene systems contain ˜60,000 atoms with dimensions of 12.50 nm×12.50 nm×10 nm and 11.10 nm×12.50 nm×10 nm, respectively. The narrow trench system, which is modelled by a CNT with a diameter of 2.45 nm and a length of 8 nm, has ˜75,000 atoms with dimensions of 10 nm×10 nm×17 nm. Periodic boundary conditions are applied in the x and y directions (flat graphene lies in the xy plane) for all the systems. The systems are non-periodic in z direction (normal to flat graphene plane). The Lennard-Jones (LJ) potential with a cutoff distance of 1.2 nm is used. The long-range electrostatic interactions are calculated using the PPPM⁵⁴. SPC/E water model is employed. The SHAKE algorithm, which maintains the rigidity of each water molecule, is applied. Sodium chloride salt solution of various concentrations (1.2M and 0.6M) is considered. The LJ parameters between atoms of graphene and water are modelled by the forcefield developed by Wu et al⁵⁵. The CHARMM forcefield⁵⁶ is used for the DNA strands.

Before starting the equilibrium and production simulations, the energy of each system was minimized for 15,000 steps. The equilibrium simulations were then performed in NPT ensemble for 2 ns at a pressure of 1 atm and a temperature of 300 K. This ensures that the water reaches its equilibrium density. The systems were further equilibrated in NVT ensemble for another 2 ns at 300 K. Temperature was maintained at 300 K by using the Nosè-Hoover thermostat^{57,58} with a time constant of 0.1 ps. The production simulations were carried out in NVT ensemble for 10 ns. Trajectories of particles were dumped every picosecond to study the structure of DNA and ions near the graphene sheet.

DFT and GW Methods: The optimized geometry of the adsorbed DNA nucleobases on flat and crumpled graphene are obtained using density functional theory (DFT) calculations. All DFT calculations are performed using the Vienna Ab initio simulation package (VASP)⁵⁹⁻⁶¹. The local density approximation (LDA)⁶² functional of Ceperley-Alder is employed based on the projector augmented wave (PAW) method⁶⁰. The cutoff energy for the plane wave basis set is 550 eV for all calculations. All ionic positions are optimized by a conjugate gradient method until the forces on each ion are less than 0.01 eV/A. The size of flat graphene and crumpled graphene model is about 12 Å×12 Å and 8.9 Å×12 Å, respectively. A vacuum separation of 40 Å between graphene and its periodic replicas is employed to eliminate the interaction between them. For accurate calculations of the electronic structures, the Brillouin zone is sampled using 18×18×1 k-point grid. The lattice parameter of flat graphene is computed to be 2.445 Å which is consistent with previous theoretical and experimental results^{63,64}.

Single-shot approximation of GW (G0W0)⁶⁵ was performed to obtain the band structures using VASP⁵⁹⁻⁶¹ Each system has been initialized using DFT with the LDA⁶⁶ exchange-correlation functional, energy cutoff of 400 eV, and Gaussian smearing of 0.05 eV. The number of total bands is set to 512 in all structures to ensure a significant number of empty bands is available as required by GW method. The energy cutoff of the response function and the number of frequency grid points are respectively set to 90 eV and 32 to control the high memory demand by the calculation of GW. Gamma-centered k-points are selected to be 6×1×1 for structures without a DNA base and 4×1×2 for structures with a DNA base. Finally, the vacuum space is maintained large enough (>20 Å) in the periodic directions to avoid unphysical interactions between the periodic images.

References corresponding to Example 1:

1. Gilliland, G., Perrin, S., Blanchard, K. & Bunn, H. F. Analysis of cytokine mRNA and DNA: detection and quantitation by competitive polymerase chain reaction. Proc. Natl. Acad. Sci. 87, 2725 LP-2729 (1990).

2. Inouye, M., Ikeda, R., Takase, M., Tsuri, T. & Chiba, J. Single-nucleotide polymorphism detection with “wire-like” DNA probes that display quasi “on-off” digital action. Proc. Natl. Acad. Sci. U.S.A. 102, 11606 LP-11610 (2005).

3. Schweitzer, B. et al. Immunoassays with rolling circle DNA amplification: A versatile platform for ultrasensitive antigen detection. Proc. Natl. Acad. Sci. 97, 10113 LP-10119 (2000).

4. Star, A. et al. Label-free detection of DNA hybridization using carbon nanotube network field-effect transistors. Proc. Natl. Acad. Sci. United States Am. 103, 921-926 (2006).

5. Hwang, M. T. et al. Highly specific SNP detection using 2D graphene electronics and DNA strand displacement. Proc. Natl. Acad. Sci. 113, 7088-7093 (2016).

6. Ping, J., Vishnubhotla, R., Vrudhula, A. & Johnson, A. T. C. Scalable Production of High-Sensitivity, Label-Free DNA Biosensors Based on Back-Gated Graphene Field Effect Transistors. ACS Nano 10, 8700-8704 (2016).

7. Sobrino, B., Brion, M. & Carracedo, A. SNPs in forensic genetics: a review on SNP typing methodologies. Forensic Sci. Int 154, 181-194 (2005).

8. Bohmann, K et al. Environmental DNA for wildlife biology and biodiversity monitoring. Trends Ecol. Evol. 29, 358-367 (2014).

9. Gonzalez-Angulo, A. M., Hennessy, B. T. J. & Mills, G. B. Future of Personalized Medicine in Oncology: A Systems Biology Approach. J. Clin. Oncol. 28, 2777-2783 (2010).

10. Rao, C. N. R., Gopalakrishnan, K. & Maitra, U. Comparative Study of Potential Applications of Graphene, MoS2, and Other Two-Dimensional Materials in Energy Devices, Sensors, and Related Areas. ACS Appl. Mater. Interfaces 7, 7809-7832 (2015).

11. Duarte-Guevara, C. et al. Characterization of a 1024×1024 DG-BioFET platform. Sensors Actuators B Chem. 250, 100-110 (2017).

12. Lin, Z el al. Carbon Nanomaterial-Based Biosensors: A Review of Design and Applications. IEEE Nanotechnology Magazine 13, 4-14 (2019).

13. Andoy, N. M., Filipiak, M. S., Vetter, D., Gutiérrez-Sanz, Ó. & Tarasov, A. Graphene-Based Electronic Immunosensor with Femtomolar Detection Limit in Whole Serum. Adv. Mater. Technol. 0, 1800186 (2018).

14. Hwang, M. T. et al. DNA Nanotweezers and Graphene Transistor Enable Label-Free Genotyping. Adv. Mater. 30, 1802440 (2018).

15. Campos, R. et al. Attomolar Label-Free Detection of DNA Hybridization with Electrolyte-Gated Graphene Field-Effect Transistors. ACS Sens. 4, 286-293 (2019).

16. Tian, M. et al. RNA Detection Based on Graphene Field-Effect Transistor Biosensor. Advances in Condensed Matter Physics, 8146765 (2018).

17. Kelley, S. O. Advancing Ultrasensitive Molecular and Cellular Analysis Methods to Speed and Simplify the Diagnosis of Disease. Acc. Chem. Res. 50, 503-507 (2017).

18. White A. K. et al. High-throughput microfluidic single-cell RT-qPCR. Proc. Natl. Acad. Sci. 108, 13999 LP-14004 (2011).

19. Costa M. C., Leitão A. L., Enguita F. J. MicroRNA Profiling in Plasma or Serum Using Quantitative RT-PCR. In: Alvarez M., Nourbakhsh M. (eds) RNA Mapping. Methods in Molecular Biology (Methods and Protocols). 1181 (Humana Press,2014).

20. Schwarzenbach, H., Hoon, D. S. B. & Pantel, K. Cell-free nucleic acids as biomarkers in cancer patients. Nature Rev. Cancer 11, 426-437 (2011).

21. Kulkarni, G. S. & Zhong, Z. Detection beyond the Debye Screening Length in a High-Frequency Nanoelectronic Biosensor. Nano Lett. 12, 719-723 (2012).

22. Gao, N. et al. Specific detection of biomolecules in physiological solutions using graphene transistor biosensors. Proc. Natl. Acad. Sci. 113, 14633 LP-14638 (2016).

23. Shoorideh, K. & Chui, C. O. On the origin of enhanced sensitivity in nanoscale FET-based biosensors. Proc. Natl. Acad. Sci. 111, 5111 LP-5116 (2014).

24. Wang, M. C. et al. Heterogeneous, Three-Dimensional Texturing of Graphene. Nano Lett. 15, 1829-1835 (2015).

25. Kang, P., Wang, M. C., Knapp, P. M. & Nam, S. Crumpled Graphene Photodetector with Enhanced, Strain-Tunable, and Wavelength-Selective Photoresponsivity. Adv. Mater. 28, 4639-4645 (2016).

26. Leem, J., Wang, M. C., Kang, P. & Nam, S. Mechanically Self-Assembled, Three-Dimensional Graphene—Gold Hybrid Nanostructures for Advanced Nanoplasmonic Sensors. Nano Lett. 15, 7684-7690 (2015).

27. Lee, M.-S. et al. High-Performance, Transparent, and Stretchable Electrodes Using Graphene-Metal Nanowire Hybrid Structures. Nano Lett. 13, 2814-2821 (2013).

28. Ni, Z. H. et al. Uniaxial Strain on Graphene: Raman Spectroscopy Study and Band-Gap Opening. ACS Nano 2, 2301-2305 (2008).

29. Choi, S.-M., Jhi, S.-H. & Son, Y.-W. Effects of strain on electronic properties of graphene. Phys. Rev. B 81, 81407 (2010).

30. Liu, T. el al. Crested two-dimensional transistors. Nature Nanotechnology 14, 223-226 (2019)

31. Sarkar, D. et al. MoS2 Field-Effect Transistor for Next-Generation Label-Free Biosensors. ACS Nano 8, 3992-4003 (2014).

32. Heerema, S., Dekker, C. Graphene nanodevices for DNA sequencing. Nature Nanotech 11, 127-136 (2016) .

33. Ma, R.-S. et al. Direct measurements of conductivity and mobility in millimeter-sized single-crystalline graphene via van der Pauw geometry. Chin. Phys. B 26, 066801 (2017).

34. Shi, Y el al. Effective doping of single-layer graphene from underlying SiO2 substrates. Phys. Rev. B 79, 115402 (2009).

35. Chen, T.-Y. et al. Label-free detection of DNA hybridization using transistors based on CVD grown graphene. Biosens. Bioelectron. 41, 103-109 (2013).

36. Adam, S., Hwang, E. H., Galitski, V. M. & Das Sarma, S. A self-consistent theory for graphene transport. Proc. Natl. Acad. Sci. 104, 18392 LP-18397 (2007).

37. Cai, B. et al. Ultrasensitive Label-Free Detection of PNA—DNA Hybridization by Reduced Graphene Oxide Field-Effect Transistor Biosensor. ACS Nano 8, 2632-2638 (2014)

38. Squirres, T. M. el al. Making it stick: convection, reaction and diffusion in surface-based biosensors. Nature Biotechnology 26, 417-426 (2008)

39. Barmi, M. R. et al. Convective Flows in Evaporating Sessile Droplets. J. Phys. Chem. B 118, 2414-2421 (2014).

40. Ljung, A. et al. Evaporation of a sessile water droplet subjected to forced convection in humid environment. Drying Technology 37, 129-138 (2019).

41. Stellwagen, E. et al. Determining the electrophoretic mobility and translational diffusion coefficients of DNA molecules in free solution. ELECTROPHORESIS 23, 2794-2803 (2002).

42. Ruggeri, F et al. Entropic Trapping of a Singly Charged Molecule in Solution. Nano Lett 18, 3773-3779 (2018).

43. Rechendorff, K. et al. Enhancement of Protein Adsorption Induced by Surface Roughness. Langmuir 22, 10885-10888 (2006)

44. Wang, Y. et al. Quantification of distinct let-7 microRNA family members by a modified stem-loop RT-qPCR. Mol. Med. Rep. 17, 3690-3696 (2018).

45. Dontschuk, N. et al. A graphene field-effect transistor as a molecule-specific probe of DNA nucleobases. Nat. Commun. 6, 6563 (2015). https://doi.org/10.1038/ncomms7563

46. Wu, G. et al. Graphene Field-Effect Transistors for the Sensitive and Selective Detection of Escherichia coli Using Pyrene-Tagged DNA Aptamer. Adv. Healthc. Mater. 6, 1700736 (2017).

47. Chen, J.-H. et al. Charged-impurity scattering in graphene. Nat. Phys. 4, 377 (2008).

48. Wang, J., Zhao, R., Yang, M., Liu, Z. & Liu, Z. Inverse relationship between carrier mobility and bandgap in graphene. J. Chem. Phys. 138, 84701 (2013).

49. Fang, J., Vandenberghe, W. G. & Fischetti, M. V. Microscopic dielectric permittivities of graphene nanoribbons and graphene. Phys. Rev. B 94, 45318 (2016).

50. Kim, J.-Y. et al. Flexible and Transparent Dielectric Film with a High Dielectric Constant Using Chemical Vapor Deposition-Grown Graphene Interlayer. ACS Nano 8, 269-274 (2014).

51. Zhang, G.-J. et al. DNA Sensing by Silicon Nanowire: Charge Layer Distance Dependence. Nano Lett. 8, 1066-1070 (2008).

52. Plimpton, S. Fast Parallel Algorithms for Short-Range Molecular Dynamics. J. Comput Phys. 117, 1-19 (1995).

53. Humphrey, W., Dalke, A. & Schulten, K. VMD: Visual molecular dynamics. J. Mol. Graph. 14, 33-38 (1996)

54. Hockney, R. W. & Eastwood, J. W. Computer Simulation Using Particles. Computer Simulation Using Particles (McGraw-Hill, 1981).

55. Wu, Y & Alum, N. R. Graphitic Carbon-Water Nonbonded Interaction Parameters. J. Phys. Chem. B 117, 8802-8813 (2013).

56. MacKerell, A. D. & Banavali, N. K. All-atom empirical force field for nucleic acids: II. Application to molecular dynamics simulations of DNA and RNA in solution. J. Comput. Chem. 21, 105-120 (2000).

57. Nosé, S. A unified formulation of the constant temperature molecular dynamics methods. J. Chem. Phys. 81, 511-519 (1984).

58. Hoover, W. G. Canonical dynamics: Equilibrium phase-space distributions. Phys. Rev. A 31, 1695-1697 (1985).

59. Kresse, G. & Furthmüller, J. Efficiency of ab-initio total energy calculations for metals and semiconductors using a plane-wave basis set. Comput. Mater. Sci. 6, 15-50 (1996).

60. Kresse, G. & Furthmüller, J. Efficient iterative schemes for ab initio total-energy calculations using a plane-wave basis set. Phys. Rev. B 54, 11169-11186 (1996).

61. Kresse, G. & Joubert, D. From ultrasoft pseudopotentials to the projector augmented-wave method. Phys. Rev. B 59, 1758-1775 (1999).

62. Ceperley, D. M. & Alder, B. J. Ground State of the Electron Gas by a Stochastic Method. Phys. Rev. Lett. 45, 566-569 (1980).

63. Lee, J.-H., Choi, Y.-K., Kim, H.-J., Scheicher, R. H. & Cho, J.-H. Physisorption of DNA Nucleobases on h-BN and Graphene: vdW-Corrected DFT Calculations. J. Phys. Chem. C 117, 13435-13441 (2013).

64. Karpan, V. M. et al. Graphite and Graphene as Perfect Spin Filters. Phys. Rev. Lett. 99, 176602 (2007).

65. Shishkin, M. & Kresse, G. Self-consistent GW calculations for semiconductors and insulators. Phys. Rev. B 75, 235102 (2007).

66. Kohn, W. & Sham, L. J. Self-Consistent Equations Including Exchange and Correlation Effects. Phys. Rev. 140, A1133-A1138 (1965).

Supplementary Information (for Example 1)

Note for Dirac voltage point determination: The measurements were repeated 6 times every two minutes for each data point (each concentration of target nucleic acids) and the Dirac point was confirmed to be stabilized when at least the last two measurements of Dirac points were same. The device was rinsed with fresh PBS every 3 measurements. Once the device and the Dirac point became stable, the Dirac points were same over many repeats of measurements. FIG. 11 shows stable Dirac points for 10 repeats of measurements over 96 min. Also, note that negative control tests showed that the signals were much smaller or negligible compared to the signal generated by the target nucleic acids (FIGS. 2F, 2H and 2I).

The Sips model (J Chem Phys 16, 490 (1948), ACS Nano 10, 8700 (2016)) is adapted to fit the DNA hybridization specifically bound on the flat and crumpled graphene surface. Single stranded DNA target molecules, Let-7b, are specifically bound on the other complimentary single stranded DNA, 22-mer probe, molecules. They form duplex DNA molecules. The Sips model is the best fit to describe the relation of the shifted Dirac point voltage responding to the absorbed DNA concentration on the crumpled graphene surface to the target concentration (C) dissolved in a buffer,

$$\vert Δ {V}_{D} ( V ) \vert = A \frac{{( C / {K}_{a} )}^{a}}{1 + {( C / {K}_{a} )}^{a}} ,$$

where A is the maximum value of Dirac point shift with all probe sites occupied, Kₐ is the equilibrium dissociation constant, and a is the characteristic parameter of the Gaussian distribution of DNA-binding energies on the graphene surface.

The Sips model is commonly applied to describe a statistical distribution of the molecular (or gas) adsorption energies on a solid surface especially when the adsorption energies of the binding sites are heterogeneous other than homogeneous.

The A value corresponds to the saturation value of Dirac point voltage when all probe molecules are fully occupied. The saturation voltage for the crumpled graphene device is about 0.122 V which is about 1.7 times larger than 0.072 V for flat. The saturation voltage becomes larger when the graphene is crumpled, in a good agreement with the capacitance simulation results. i.e., the voltage is inversely proportional to the capacitance and the capacitance of the crumpled is reduced in a half of the value of the flat.

The association constant a characterizes the energy distribution of the DNA adsorption isotherm on the surface. The a value is in a range from 0 to 1. When a=1, the Equation turns into the Langmuir adsorption isotherm, in which all the DNA-binding sites have the same binding energy. If a decreases, the distribution curve shows a transition from a steep slope to a low slope as the target concentration increases. For the limit of a=0, it results in a constant value. From the crumpled graphene results, a=0.2 reflects the broader

DNA-binding energy distribution on the crumpled as compared with 0.436 for the flat case. It assumes that the broad energy distribution must be correlated with heterogeneous DNA-binding sites, such as the deep valleys, the slopes, or the peaks of the crumpled surface.

The dissociation constant Ka is a strong relationship with the binding DNA length. The Kₐ value decreases exponentially as the adsorbed molecular size increases (ref. ACS Nano 2016 10 8700). From the data set, two Ka values are very similar between the crumpled and flat devices.

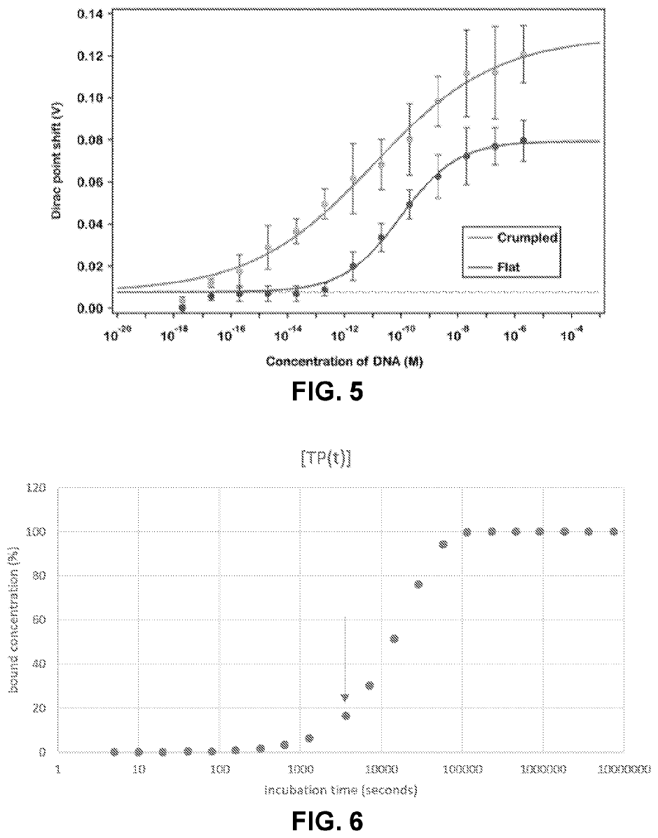

As shown in FIG. 5, the detection limit is actually shifted from a femto-Molar for the flat down to an atto-Molar for the crumpled. The Sips fitting parameters A and a describe, with a statistical method, that the crumpling effects enhance the capacitance and also lower the detection responding slope so that the crumpled-graphene system can detect the adsorbed DNA molecules in an atto-molar scale concentration.

Consideration of convection-diffusion-reaction model: Taking into account convection-diffusion-reaction considerations, evaporation induced convection and surface roughness effect on molecular absorption may facilitate the transport of nucleic acids to the graphene surface, reducing the diffusion-reaction time significantly and contributing to the high-sensitivity detection. Also, the surface roughness of crumpled graphene may influence the molecular reaction process, compared to a flat graphene

In general, while a small volume of water droplet is placed on a solid substrate at a room temperature, the water droplet evaporates and drives convention flow. The convection flows down from the top surface of the droplet to its bottom solid-water surface and then flows up from the edge to the top. The convection flow rates depend on temperature and humidity. Due to convective flow, the molecules at the central region move along downstream to the bottom surface and spread over the surface in a radial direction. The flow speed varies inside the droplet. The speed is highest at the top central region and becomes much slower on the bottom region (where diffusion wins). The typical value ranges from 0.1 to 100 um/s at room temperature (J. Phys. Chem. B 118, 2414-2421 (2014)). In other words, the molecules approach to the surface fast at the central region and slow down on the surface where diffusive transport is dominant (Drying Technology 37, 129-138 (2019)).

Considering that target molecules are across r=3 mm, radius of water droplet, the diffusion time is proportional to r/2D, D is diffusion constant, and convective time is proportional to the evaporation rate, Q, is about 0.33 μl/min inside the probe station. Peclet number, Pe, can characterize the mass transport as diffusion-limited or reaction-limited (Nature Biotechnology 26, 417-426 (2008)). For convection and diffusion (D=100 μm²/s) (ELECTROPHORESIS 23, 2794-2803 (2002)), If Pe=diffusive time/convective time is >>1, reaction limit dominates the system while if Pe<<1, diffusion limit dominates In our case, Pe is ˜10, which means that chemical reaction occurs slowly while target molecules are supplied relatively fast to the surface. In the reaction-limited, the binding reaction is the major time obstacle to collect all target molecules on the sensor area.

By applying Langmuir kinetics to assume target bindings on the reactive surface, the target concentration can be estimated by:

$$\frac{\partial [ TP ( t ) ]}{\partial t} = {k}_{o n} [ T ] ( {P}_{m} - [ T P ( t ) ] ) - {k}_{off} [ T P ( t ) ]$$

where Pₘ is the probe concentration, [T] target concentration, [TP] target-probe complex concentration, kₒₙ is an associate rate constant, k_{off} is a dissociation rate constant. For chemical reactions when all molecules can diffuse to the surface, the equation above can be solved:

$$[ T P ( t ) ] = {P}_{m} \frac{[ T ] / {K}_{D}}{1 + [ T ] / {K}_{D}} ( 1 - {e}^{- ( {k}_{o n} [ T ] + {k}_{off} ) t} )$$

where K_{D}=k_{off}/kₒₙ is the equilibrium dissociation constant. Given parameters, K_{D}=1 pM (from Sip's fitting results), Pₘ=1×10³/um² (ACS Nano 10, 8700 (2016)), kₒₙ is 10⁶ M⁻¹ s⁻¹, and K_{off}=10⁻⁵ s⁻¹, as shown in the plot of [TP(t)] vs time, the equilibrium time reaches to ˜10⁵ seconds (16 hours) for sub pico-molar target concentration. Since the equilibrium time decreases as the concentration increases, the graphene sensor is limited to pico-molar sensitivity within one-hour equilibrium state (Nano Lett. 5, 803 (2005)). Before reaching the equilibrium state, the one-hour practical incubation time indicated by arrow, about 16.5% of target DNA concentration molecules can be bound on the surface. Graphene sensors are experimentally able to detect them (Nano let. 18, 3509 (2018)).

The surface roughness of crumpled graphene may also influence the molecular adsorption process, compared to a flat graphene. Crumpled graphene forms randomly oriented valleys-and-peaks surface. The RMS roughness is about 500nm between valleys and 300 nm for their depth (Nano Lett. 15, 7684-7690 (2015)). When the molecular size is larger than 500 nm, conformational entropic trap holds the molecules inside the valley. On the other hand, when the size is much smaller, the molecules move “relatively” freely inside and outside of the rough surface. The molecules face increased interactions, such as Van der Waals, electrostatic force, and hydrophobicity. Thus, they stay in the valleys for longer than typical diffusion time scale (Nano Lett. 18, 3773-3779 (2018)). Ruggeri et al, claimed the well depth of 330 nm gives up to 5 k_{B}T configurational free energy barrier, W, and the molecular residence time is proportional to exp(W/k_{B}T). The molecules stay longer by 10²× of diffusion time scale. Practically, molecular adsorption rate is increased with increasing surface roughness (Langmuir 22, 10885-10888 (2006)).

Therefore, collectively evaporation induced convection and surface roughness effect on molecular absorption possibly facilitate the transportation of nucleic acids to the graphene surface and may reduce the diffusion-reaction time significantly and contributed to the high-sensitivity detection.

DNA Adsorption to the Graphene Surface

The interaction energies between the DNA and crumpled graphene are calculated for different configurations of DNA. The equilibrium energies for the DNA in the concave, convex, across and flat regions are −532.187 kcal mol⁻¹, −467.484 kcal mol⁻¹, −416.308 kcal mol⁻¹ and −500.383 kcal mol⁻¹, respectively. FIG. 18 shows the evolution of the interaction energies as the DNA strand binds onto the graphene surface.

We investigated the DNA adsorption onto the graphene by plotting the area per nucleotide (packing) for the concentrations used in this study in FIG. 19 assuming all DNA molecules formed a monolayer at the graphene interface. As shown, for the concentrations higher than 1 nM, the area per nucleotide is lower than the area occupied by a DNA nucleotide (based on the size of one nucleotide) indicating an extreme packing if the molecules were hypothetically adsorbed as a monolayer next to graphene. Therefore, DNA adsorption saturation must take place for high concentrations (˜1 nM and above) and the additional molecules are simply accumulated in layers away from graphene.

Dirac point shift mechanisms for different concentrations: We studied the Dirac point shift for a range of different concentrations to identify the dominant mechanisms by which the shift takes place. First, we excluded the effect of the band gap and calculated the shift solely based on the charge transfer from the unscreened DNA molecules using

$$Δ {V}_{D} = \frac{e {N}_{D N A}^{u n s c r e e n e d}}{{C}_{T}} .$$

By matching the experimental ΔV_{D}, charge transfer (N_{DNA}^{unscreened}) can be extracted. The ratio of the transferred charge to the total available DNA charge in the solution is plotted for different concentrations in FIG. 21A. As shown, for the crumpled graphene, the charge transfer is higher than that of a flat graphene (indicating less screening in crumpled graphene) for the intermediate range of concentrations. In addition, the required charge transfer exceeds the maximum available charge (>100%) for low concentrations indicating that charge transfer by its own is not the only mechanism responsible for the change in the carrier charge density. Next, we assume a constant change in the charge carrier density corresponding to a Dirac point shift of −0.042 V due to the band gap change (we justify the band gap opening in the next section of this supplementary information). In FIG. 21B, with the band gap included, the % of charge transfer is estimated by matching the experimental shifts. As shown, for the crumpled graphene, for the two highest concentrations, the charge transfer is almost zero as the band gap is the dominant contributor to the shift. In FIG. 21C, we plotted the individual contribution of charge transfer and bandgap (note that due to the complexity of band gap opening, we assume a constant Dirac point shift due to band gap opening). In FIG. 21D, the experimental data is replotted where we divided the concentrations into three regions. <200 aM region where band gap opening is dominant, >200 aM and <1 nM region where the charge transfer becomes significant in addition to the band gap and >1 nM region where the charge transfer is dominant while DNA adsorption saturation on graphene takes place.

Modeling of Dirac point shift for band gap opening:

The shift is directly obtained from

$$Δ {V}_{D} = \frac{e {N}_{D N A}^{u n s c r e e n e d}}{{C}_{T}}$$

without including any effect from bandgap. However, for the ultralow concentrations (e.g., 2 aM), the shifts are too small to be noticed without including the effects from bandgap opening (FIGS. 22C-22D and FIGS. 21A-21D). The carrier charge density change of graphene due to a bandgap change after adding DNA molecules can be obtained from

$$Δ {n}_{band gap} = \frac{1}{e ρ} ( \frac{1}{{μ}^{D N A}} - \frac{1}{{μ}^{no DNA}} ) ,$$

where e is the elementary charge, and ρ is the resistivity. Wang et al.¹ showed that electronic mobility (μ) in graphene decreases with increasing bandgap where mobility is obtained empirically from

$$μ = 0 \text{.114} \times {10}^{4} {E}_{g}^{- \frac{3}{{2}^{}}}$$

(E_{g} in eV and μ in cm² V⁻¹ s⁻¹) Since the bandgap changes are local, the global (macroscopic) change in the charge carrier density is estimated by Δn_{global}=b Δn_{bangap}, where b is the area fraction of the affected regions. For the 2 aM concentration, assuming a bandgap change from 0.4224 eV to 1.7641 eV in 10⁻⁷% of the crumpled graphene where each DNA nucleotide affects at least an area of 39.9 nm² (see the calculation below in the next section), the shift is noticeable and is estimated to be ˜5 mV

$$( using {Δ V}_{D} = \frac{{e Δ n}_{global}}{{C}_{T}} )$$

as shown in FIG. 22E. 39.9 nm²/nucleotide (an area with a diameter of 7.1 nm) is relatively large compared to the size of a single DNA nucleotide. To ensure the bandgap change is indeed long-range, we investigated the effect of graphene size on the bandgap opening due to a single DNA base. As shown in FIG. 23, we first considered one unit cell (width of ˜12 and length of ˜12 Å) and computed the bandgap of crumpled graphene in the presence of A-base (with the orientation 1 as indicated in FIG. 28). Then, keeping the single A-base, we increased the number of the crumpled graphene unit cells to 2, 3 and 4. For example, a 4-unit cell is a supercell with a width of ˜12 Å and a length of ˜48 Å. Note that the width of all the cells is ˜12 Å and only the length is varied. Based on this study, we observe that the bandgap for the case of 4-unit cell is about 60% of that of a single unit cell which is still a significant bandgap value. This supports our hypothesis that the local change of bandgap is long-range and influences the electronic properties of graphene globally.

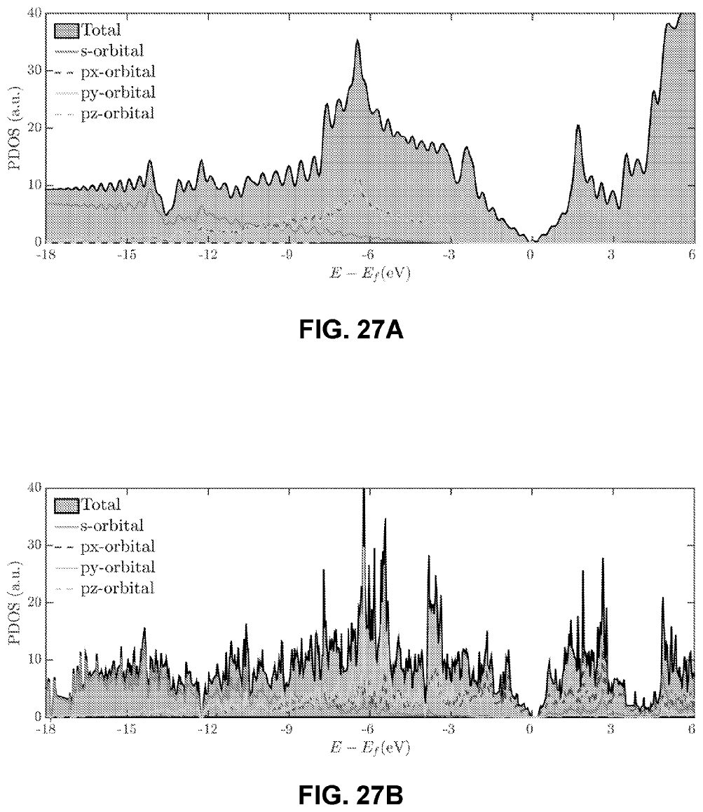

Crumpling graphene by introducing 1D periodic ripples is found to produce a bandgap opening²⁻⁴. The opening of bandgap is attributed to the change in graphene curvature introducing quantum confinement with distinct electronic structures compared to the pristine/flat graphene³. In pristine graphene, the C—C bond length is ˜1.41 Å for all carbon atoms with an angle of 120° which results in sp² hybridization. When graphene is crumpled, the bond length and the angles vary across the graphene. The optimized structure that we obtained using DFT shows that the C—C bond length has a value of 1.41-1.55 Å depending on the local curvature. In addition, the angles between the carbon atoms is found to be either 120° or 110°. The bond length of 1.55 Å and angle of 110° resemble the structure for sp³ hybridized C atoms^{5,6}. Thus, crumpled graphene contains sp³ and sp² hybridization between C atoms. This is expected since the C atoms are not in the same plane due to crumpling. Further, the partial density of states of the pristine graphene (see FIGS. 27A-27B) shows the px and pz orbitals having sp² hybridization. By analyzing the partial density of states of the crumpled graphene (see FIGS. 27A-27B), we observe overlapping between all three p orbitals (i.e., px, py and pz) and between only two of them at a given energy level supporting the presence of both sp³ and sp² hybridizations. Introducing sp³ bonds produces strongly localized hybridization⁷⁻⁸ resulting in a bandgap opening. It should be noted that changing the degree of hybridization (i.e., electronic orbitals overlap) via changing the bonding interactions (e.g. bond length and angle) is found to play a significant role in controlling the conduction and valence energy band levels which leads to altered bandgaps in different materials⁹⁻¹³.

Calculation of area needed for low number of molecules

$${Δn}_{b a n d g a p} ( local ) = \frac{1}{e ρ} ( \frac{1}{{μ}^{D N A}} - \frac{1}{{μ}^{no DNA}} ) , μ = 0.114 \times 1 {0}^{4} {E}_{g}^{- \frac{3}{{2}^{}}}$$

$$e Δ {n}_{b a n d g a p} ( local ) = \frac{1}{∼ 100 Ω} ( \frac{1.764 {1}^{\frac{3}{2}}}{0 \text{.114} \times {10}^{4}} - \frac{0.422 {4}^{\frac{3}{2}}}{0 \text{.114} \times {10}^{4}} ) Vs {cm}^{- 2} = 1.815 \times 1 {0}^{- 5} C {cm}^{- 2}$$

$$Δ V ( local ) = \frac{e Δ n ( local )}{C ( local in concave )} = \frac{1.815 \times 1 {0}^{- 5} C {cm}^{- 2}}{0.16 \times 1 {0}^{- 5} f {cm}^{- 2}} = 1.1343 \times 1 {0}^{4} V or 1.1343 \times 1 {0}^{7} mV$$

$$To get 0.1 mV shift , fraction = \frac{0.1 mV}{1.1343 \times 1 {0}^{7} mV} = 8.8 \times 1 {0}^{- 9} or ∼ {10}^{- 7}$$

DNA Orientations in Ab Inito Calculations:

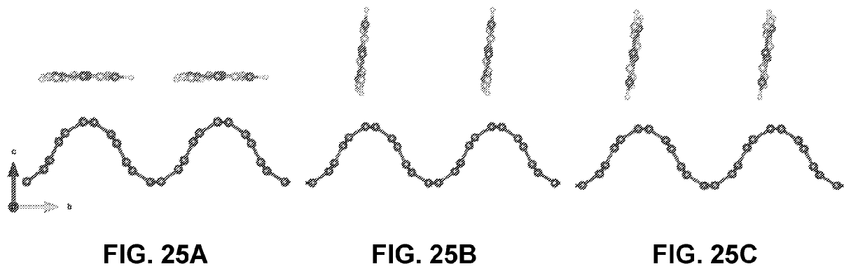

In orientation 1 and orientation 2 (see FIGS. 24A-24C and FIGS. 25A-25C), the plane of the base ring is parallel and perpendicular to the graphene surface, respectively. The plane of the base ring in orientation 3 is the same as in orientation 2. However, the nucleobase is rotated 180° as shown in FIGS. 24A-24C and FIGS. 25A-25C.

Since GW is computationally expensive, we only performed GW for orientation 1 on all the graphene surfaces and orientation 2 on the crumpled armchair graphene. Comparing the DFT and GW bandgaps of crumpled graphene, we note that the DFT bandgaps for orientations 1 and 2 are almost identical. However, the GW bandgaps are different for different orientations. To understand the interactions of the different orientations, we computed the interfacial charge density. The charge density difference of the system (i.e., graphene and nucleobase system), Δρₛ, is defined as

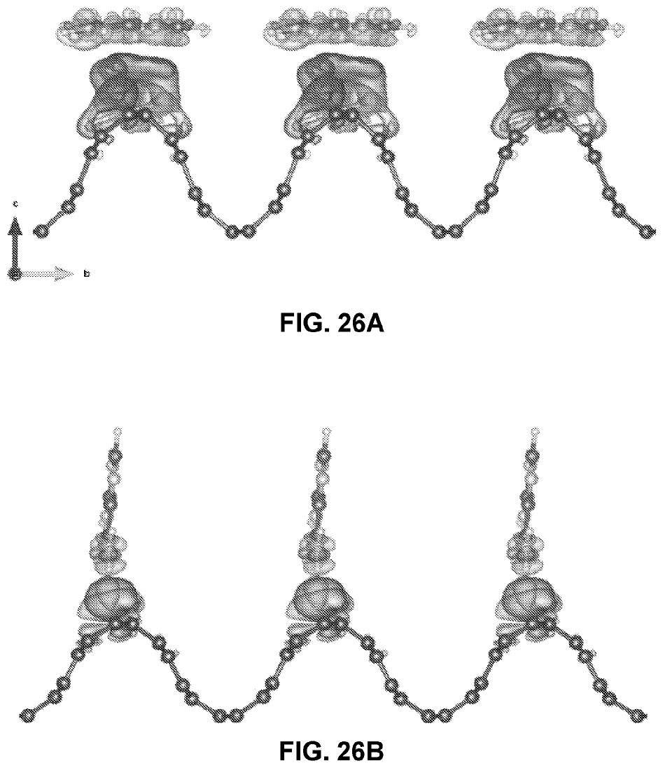

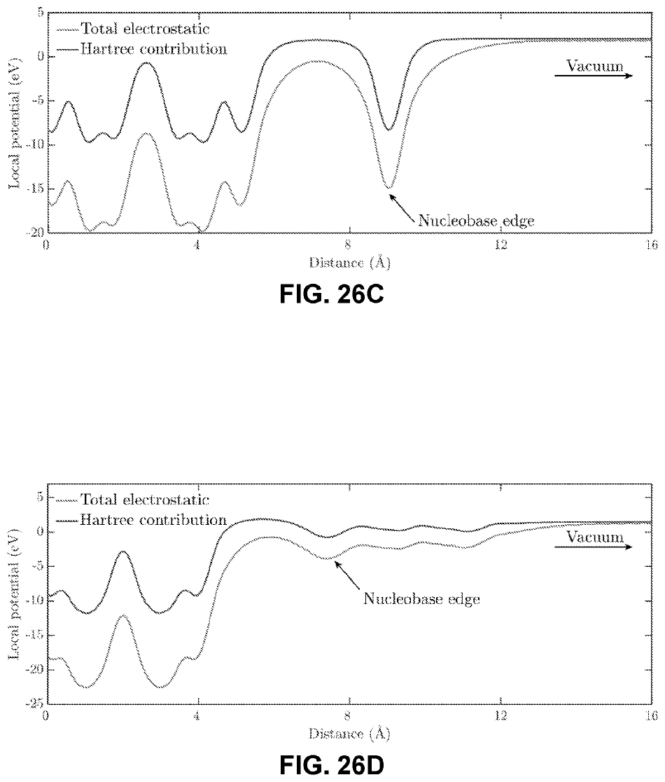

Δρₛ=ρₛ−ρ_{g}−ρₙ

where ρₛ is the charge density of the full system including both the crumpled graphene and the nucleobase in the unit cell, ρ_{g} is the charge density obtained by simulating the crumpled graphene without the nucleobase, and ρₙ is the charge density obtained by simulating the nucleobase without the crumpled graphene. Δρ_s represents the interfacial charge density due to the adsorption of the nucleobase onto the crumpled graphene. As shown in FIGS. 26A-26D, when the nucleobase is placed parallel to the crumpled graphene, the interfacial charge density is distributed along the nucleobase atoms. However, when the nucleobase is perpendicular to the crumpled graphene, the charge density is predominantly on the lower edge of the nucleobase experiences. Therefore, parallel orientation has a larger interfacial area with more Coulombic interactions (all atoms in the nucleobase directly interact with the crumpled graphene) compared to the perpendicular orientation (only the atoms towards the lower edge of the nucleobase have a direct interaction with the graphene surface). The different charge distributions and stacking lead to different electronic interactions for each orientation^{14,15}. Further, FIGS. 26A-26D show the local electrostatic and local Hartree potential of each orientation in the non-periodic direction (c-direction shown in FIG. 26A). At the interface, for orientation 1, there is a high energy barrier compared to orientation 2, which results in a higher bandgap for orientation 1. Different interactions in these orientations lead to different band gaps as captured by the GW method which accounts for Coulombic interactions using a many-body approach^{16,17}.

Raman Spectroscopy Analysis on VHS Substrate:

As shown in the FIG. 1D and FIGS. 10A-10B, we had Raman spectroscopy. However, as polystyrene (PS) substrate has Raman peak close to graphene G-peak, so the Raman peak marked as “G” in the plot is actually ‘graphene G peak+one of PS’. To observe the G peak without the peak from PS, we prepared flat and crumpled samples on VHB substrate (because VHB does not have Raman peak close to graphene G peak). The following is the sample preparation protocol;

1. Transfer graphene on a PDMS stamp

2. Transfer graphene onto VHB substrate.

a. For Flat sample: (VHB tape was put on a slide glass because the soft VHB nature made hard the contact printing process.)

b. For crumpled sample: 100% prestrain was applied in x- and y-axes to apply same amount of prestrain as crumpled graphene on PS substrate.

The results are shown in FIGS. 7A-7E.

Based on 2D/G intensity ratio (FIG. 7D), both crumpled and flat samples showed larger than 2. FWHM of 2D peak was analyzed, and in both samples, FWHM of 2D peak indicates monolayer graphene. (For monolayer graphene, FWHM<30 cm⁻¹, Nano Lett. 2007, 7, 2, 238-242). VHB has a Raman peak at ˜2670 cm−1 and in crumpled sample, and it was overlapped with graphene's 2D peak. That would have made crumpled graphene sample's FWHM value larger than flat sample. 2D peak center was also analyzed to be larger from crumpled sample (2636.4 cm−1 vs. 2628.2 cm−1) with similar reason. . Based on our Raman spectroscopy analysis, both crumpled and flat graphene samples were monolayer (potentially with small number of bilayer islands due to the graphene growth process, CVD). In addition, since crumpled graphene is three-dimensional structure, out-of-plane structures may interact or even touch each other, but spectral analysis does not indicate apparent evidence of local folding/touching/interacting of graphene structures. We have added this result in FIGS. 10A-10F. Also there is a previous report that the graphene remained monolayer after the same crumpling process (Adv. Mater., 28: 4639-4645). (n=5)

References for the Supplementary Information

1. Wang, J. Y., Zhao, R. Q., Yang, M. M., Liu, Z. F. & Liu, Z. R. Inverse relationship between carrier mobility and bandgap in graphene. J. Chem. Phys. 138, doi:10.1063/1.4792142 (2013).

2. Bai K K, Zhou Y, Zheng H, Meng L, Peng H, Liu Z, Nie J C, He L. Creating one-dimensional nanoscale periodic ripples in a continuous mosaic graphene monolayer. Physical review letters. 2014 Aug. 18; 113(8):086102.

3. Lee J K, Yamazaki S, Yun H, Park J, Kennedy G P, Kim G T, Pietzsch O, Wiesendanger R, Lee S, Hong S, Dettlaff-Weglikowska U. Modification of electrical properties of graphene by substrate-induced nanomodulation. Nano letters. 2013 Jul. 19; 13(8):3494-500.

4. Lim H, Jung J, Ruoff RS, Kim Y. Structurally driven one-dimensional electron confinement in sub-5-nm graphene nanowrinkles. Nature communications. 2015 Oct. 23; 6:8601.

5. Sofo J O, Chaudhari A S, Barber G D. Graphane: A two-dimensional hydrocarbon. Physical Review B. 2007 Apr. 10; 75(15):153401.

6. Boukhvalov D W, Katsnelson M I, Lichtenstein A I. Hydrogen on graphene: Electronic structure, total energy, structural distortions and magnetism from first-principles calculations. Physical Review B. 2008 Jan. 22; 77(3):035427.

7. Abrasonis G, Gago R, Vinnichenko M, Kreissig U, Kolitsch A, Möller W. Sixfold ring clustering in s p 2-dominated carbon and carbon nitride thin films: A Raman spectroscopy study. Physical Review B. 2006 Mar. 24; 73(12):125427.

8. Zhang Y, Shang J, Fu W, Zeng L, Tang T, Cai Y. A sp 2+ sp 3 hybridized carbon allotrope transformed from AB stacking graphyne and THD-graphene. AIP Advances. 2018 Jan. 30; 8(1):015028.

9. Miglio A, Heinrich C P, Tremel W, Hautier G, Zeier W G. Local bonding influence on the band edge and band gap formation in quaternary chalcopyrites. Advanced Science. 2017 September; 4(9):1700080.

10. Jaffe J E, Zunger A. Theory of the band-gap anomaly in AB C 2 chalcopyrite semiconductors. Physical Review B. 1984 Feb. 15; 29(4):1882.

11. Khoa D Q, Nguyen C V, Bui L M, Phuc H V, Hoi B D, Hieu NV, Nha V Q, Huynh N, Nhan L C, Hieu N N. Opening a band gap in graphene by C—C bond alternation: a tight binding approach. Materials Research Express. 2019 Jan. 9; 6(4):045605.

12. Prasanna R, Gold-Parker A, Leijtens T, Conings B, Babayigit A, Boyen H G, Toney M F, McGehee M D. Band gap tuning via lattice contraction and octahedral tilting in perovskite materials for photovoltaics. Journal of the American Chemical Society. 2017 Aug. 4; 139(32):11117-24.

13. Peng X, Velasquez S. Strain modulated band gap of edge passivated armchair graphene nanoribbons. Applied Physics Letters. 2011 Jan. 10; 98(2):023112.

14. Park C, Atalla V, Smith S, Yoon M. Understanding the Charge Transfer at the

Interface of Electron Donors and Acceptors: TTF-TCNQ as an Example. ACS applied materials & interfaces. 2017 Aug. 2; 9(32):27266-72.

15. Fernandez A C, Castellani N J. Dipole moment effects in dopamine/N-doped-graphene systems. Surface Science. 2020 Mar. 1; 693:121546.

16. Hybertsen MS, Louie SG. Electron correlation in semiconductors and insulators: Band gaps and quasiparticle energies. Physical Review B. 1986 Oct. 15; 34(8):5390.

17. Johnson K A, Ashcroft N W. Corrections to density-functional theory band gaps. Physical Review B. 1998 Dec. 15; 58(23):15548.

### EXAMPLE 2

### Detection of DNA Amplification

The biosensors, FETs, systems and methods described herein can be used for the sensitive detection of an amplification product, such as polynucleotide activation. For example, this example relates to a high sensitivity graphene field effect transistor (gFET) for detection of DNA amplification.

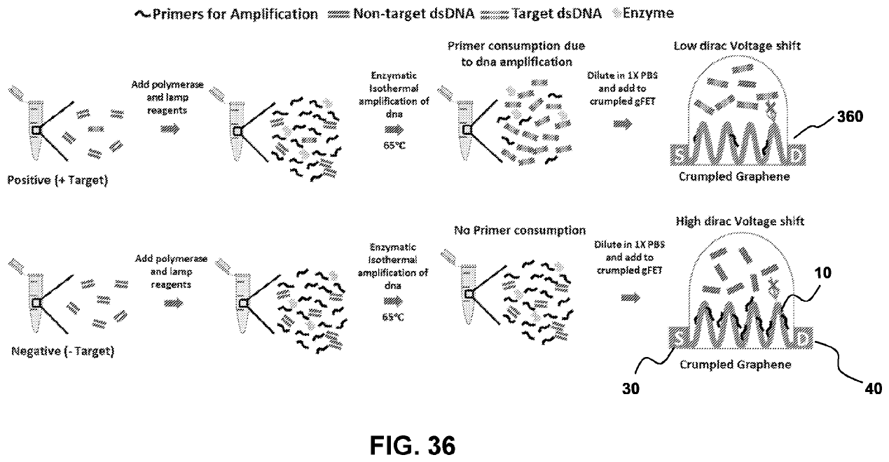

Any of the biosensors described previously, including as illustrated in FIGS. 1A, 1B, 33A and 33B correspond to the systems of this example, including a FET 360 with source electrode 30, drain electrode 40, and channel layer 10 there between that can have a crumpled configuration (FIG. 36)). The channel layer 10 can have a receiving surface 11 (see, e.g., FIG. 1A) that forms part of sample reservoir 80 that holds an amplifiable sample solution 70. An electrical detector 91 in electrical contact with the FET (or the sample solution in detectable contact with the FET), can be part of power source 90 (FIG. 33B) or a separate component (FIG. 1A).

In this Example, we use Bst polymerase in a Loop Mediated Isothermal Amplification (LAMP) reaction combined with target specific primers and crumpled graphene field effect transistors (gFET) to electrically detect the amplification by sensing the reduction in primers. Graphene is known to adsorb single stranded DNA due to noncovalent π-π bonds, but not double stranded DNA. Other materials that mediate a similar reaction can be used in place of graphene, as described herein. Our approach does not require any surface functionalization and allows the detection of primer concentrations at the endpoint of reactions. We chose crumpled gFET over the conventional flat gFET sensors due to their superior sensitivity as recently demonstrated (see, e.g., U.S. Pat App. No. 62/982,801 filed Feb. 28, 2020 titled “ULTRASENSITIVE BIOSENSOR USING BENT AND CURVED FIELD EFFECT TRANSISTOR BY DEBYE LENGTH MODULATION” (Atty Ref 338264: 7-20P US)). We were able to detect the endpoint of amplification reaction with starting concentrations down to 8 zeptomolar in 90 minutes including the time of amplification and detection. With its high sensitivity and small footprint, our platform will help bring complex lab based diagnostic and genotyping amplification assays to the point-of-care.

Rapid and accurate detection of infection causing pathogens such as E. coli and others remain a challenge in healthcare¹. State of the art for sensitive and specific detection of pathogens usually relies on their genomic DNA amplification using techniques such as Polymerase Chain Reaction (PCR) or Loop Mediated Isothermal Amplification (LAMP) which require expensive optical readout instrumentation for measuring the fluorescence signal from intercalating DNA binding dyes². Towards optics-free platforms, approaches using carbon-nanotube and silicon nanowire field effect transistors for electrical and label-free DNA ‘hybridization-based’ detection have been previously reported^{3,4,5,6}. However, these DNA hybridization based techniques often have a lower a limit-of-detection (LOD) (picomolar to femtomolar range)⁶, as compared to the LOD of optics based DNA amplification techniques (low attomolar range)^{7,8}. CMOS compatible ion sensitive field effect transistors (ISFET) arrays have also been used for hybridization-based DNA detection but with limit of detection usually in nanomolar range⁹. ISFETs have also been used for label-free electrical detection of DNA amplification by detecting pH changes during an amplification reaction^{10,11}. However, these platforms often require special low buffer capacity reactions for detecting signals and have inferior performance (LOD>10 aM¹⁰) as compared to optical readout systems.

Towards electrical detection of biomolecules, graphene Field-effect transistor-based biosensors (gFET) offer many potential advantages such as large surface-to-volume ratio, high carrier mobility and low cost¹². Several gFET based platforms offering high sensitivity, low cost, and high throughput detection using diverse sensing methods such as electrochemical¹³, back-gated G-FETs¹⁴, and liquid-gated G-FETs¹⁵⁻¹⁸ have been reported in the literatures. The semiconductor-dielectric interface inside the conventional ISFET is not accessible for biomolecule functionalization and usually the analyte in the sample will be adsorbed/attached to the gate oxide and will modulate the capacitance and electric field across it¹⁶. In such devices, the gate oxide should be thin to better modulate the electric signal and increase the sensitivity of the device while also being thick enough to reduce the gate leakage current and increase signal to noise ratio¹⁹. In contrast, graphene electrolyte-gated FET biosensors can overcome these drawbacks as the transistor channel is formed by a single two-dimensional (2-D), one-atom-thick carbon layer, which can be left accessible for direct functionalization or adsorption with biomolecules. Hence, the local gating effect is much more effective than conventional devices²⁰⁻²⁶. Many studies have demonstrated the use of gFET sensor for biomolecules detection, including protein, DNA, and bacteria²⁷. To enable specific binding, GFETs can be functionalized with single-stranded probe DNA for detection of specific target DNA through DNA-DNA hybridization with complementary sequences. Using this DNA hybridization based approach, gFETs typically offer a limit of detection (LOD) ranging from 100 pM to 1 fM^{15,17,28-30}. A further improvement of LOD, up to 25 aM, was recently reported by optimizing the bio-FET channel and using a large-area in-plane gate surrounding the graphene channel that allows a uniform distribution of potential inside the water droplet and a uniform gating field¹⁹.

The net electrostatic effect of a charged molecule in the solution containing different ions is measured in terms of Debye length with characteristic thickness of less than 1 nm in physiological solutions¹⁹. Outside the Debye length, charge carriers are increasingly electrically screened. By increasing the Debye length, the sensitivity of the G-FETs to detect target DNA can be enhanced because more sequence length of DNA strand is within the Debye length and thus more electric charge is induced near the graphene surface. This will result in a higher change in the electric conductance of the graphene channel. Previous computational studies have shown that curved morphologies, such as the concave regions of nanowire sensors, can affect the Debye length³¹. Studies have also reported that crumpled graphene, which has concave and convex deformations at the micro and nano scale can be fabricated using pre-strained thermoplastics and relieving stress to induce buckle delamination of graphene³². The mechanically tunable crumpled graphene has already been explored in several applications such as stretchable photosensors³³ and strain gauges³⁴. Its application in biosensing has also been recently reported with hybridization based-DNA detection with improved sensitivity³⁶. The sensitivity enhancement in crumpled graphene in this recent study was attributed to the nanoscale deformations, specifically the concave regions, that decrease the charge screening of the nucleic acid molecules by increasing the Debye length in the ionic solution³⁶. In addition, the crumpled graphene could form a band gap in the deformed regions further allowing for an exponential source-drain current change from a small number of charges³⁶. However, despite its improved sensitivity, this hybridization-based method still requires surface functionalization of the graphene channel and thus can lead to complications in fabrication process.

In this example, we show that crumpled graphene FETs can be used to detect physiosorbed single stranded DNA (ssDNA) molecules (as compared to double stranded DNA product) on its surface and use this for detecting enzymatic amplification by monitoring the reduction in primer (ssDNA) concentration in a reaction. Unlike double stranded DNA (dsDNA), ssDNA can be strongly adsorbed on the graphene FET surface through noncovalent stacking interaction between the hexagonal cells of graphene and the aromatic ring structure of unpaired nucleobases³⁷⁻⁴⁰. In our platform, we use this discrimination power of crumpled graphene coupled with primer (ssDNA) consumption in enzymatic loop mediated isothermal amplification(LAMP)⁴¹ to detect E. coli DNA down to zeptomolar (zM) concentrations in end-point LAMP reactions. gFET Signal generated from primers is reduced only if the specific target is present and amplification occurs where primers are consumed during amplification and become a part of formed dsDNA. In contrast, the dsDNA produced in the amplification does not produce any significant shift in the Dirac voltage. LAMP was chosen as our assay reaction as it uses a robust strand-displacement Bst polymerase and six sequence specific primers compared to the 2 used in PCR⁴². Moreover, since it is an isothermal reaction, the instrumentation demands are easier. LAMP is also known to be highly specific and sensitive and hence offers single molecule (attomolar) sensitivity for detecting target DNA⁴³. We expect our platform, with its electrical, label-free and surface modification-free detection of enzymatic amplification, will allow translation of complex and sensitive lab-based amplification assays to truly point-of-care and small footprint detection devices.

Results and discussion: Process overview and device characterization for physisorption of ssDNA and dsDNA: The approach for using crumpled graphene FET sensor for detection of LAMP reaction is illustrated in FIG. 36. Amplification reagents, including target DNA specific primers and Bst polymerase enzyme, are added to the target DNA (or negative control) in a reaction tube and isothermal DNA amplification is performed at 65° C. for 1 hour. After 1 hour, the complete reaction mix is diluted in 1× PBS and added to the crumpled graphene FET for 15 minutes to allow physisorption of molecules. After 15 minutes, the samples are rinsed in 1× PBS again and the Dirac point shifts due to the physiosorbed molecules are measured. Details on dilution and measurements can be found in the methods section. In the LAMP reaction, primer molecules which are ssDNA and present in excess in the reaction, bind to the target DNA and with the help of polymerase enzyme, copies of the target DNA are generated. In this process, the ssDNA primers are consumed and converted to dsDNA target copies⁴⁴. Within 1 hour, this results in consumption of primers and hence reduction of single stranded DNA (ssDNA) concentration only in reactions where the specific target DNA was present. Since the formed double stranded DNA (dsDNA) during amplification does not bind to crumpled graphene as strongly due to paired DNA bases which causes the π-π stacking sites to be inside the helix⁴⁰, we see an overall lower Dirac voltage shift for samples containing the target DNA compared to negative control samples.

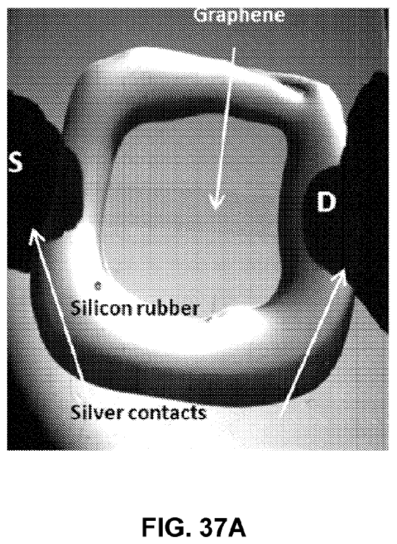

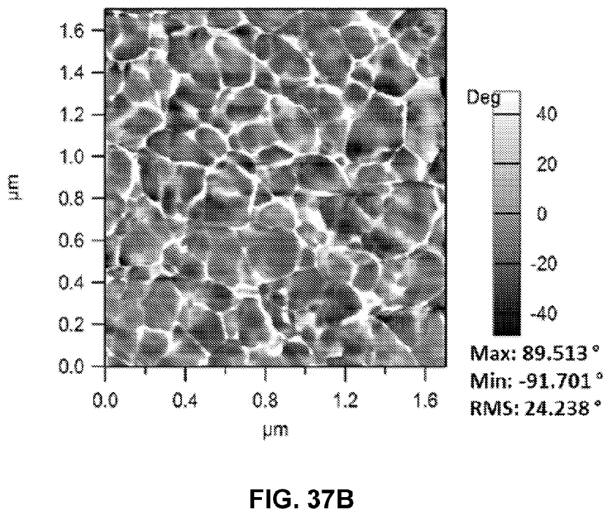

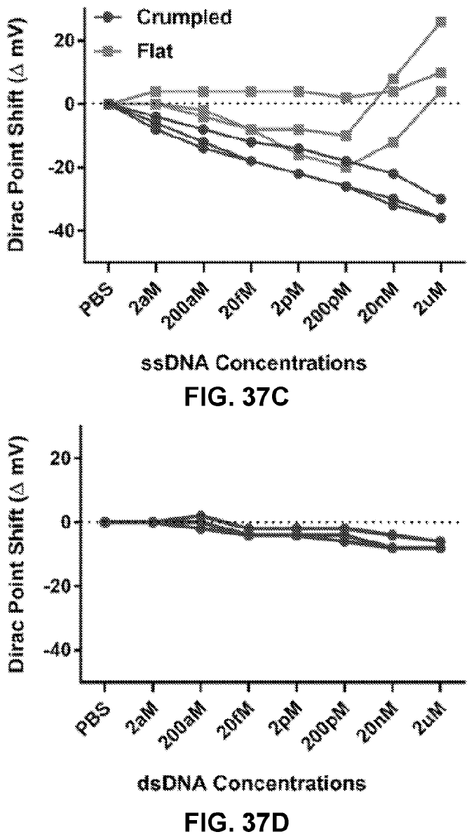

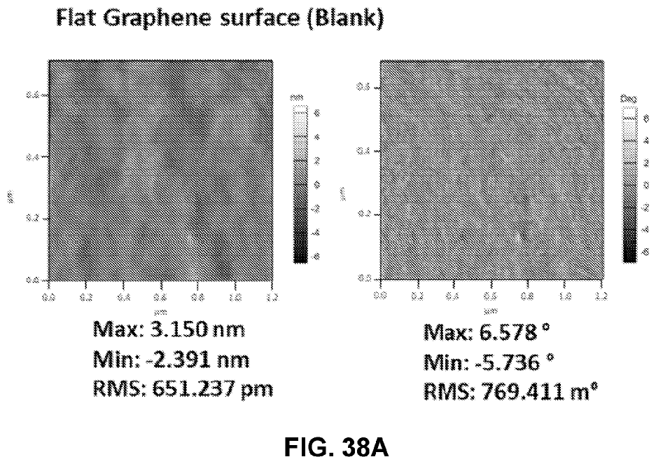

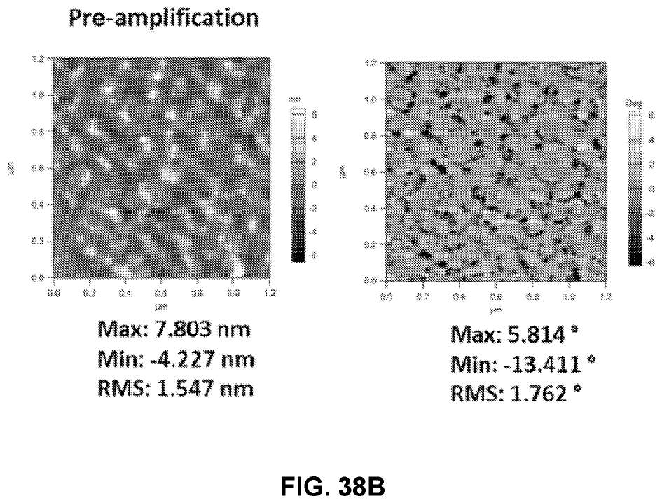

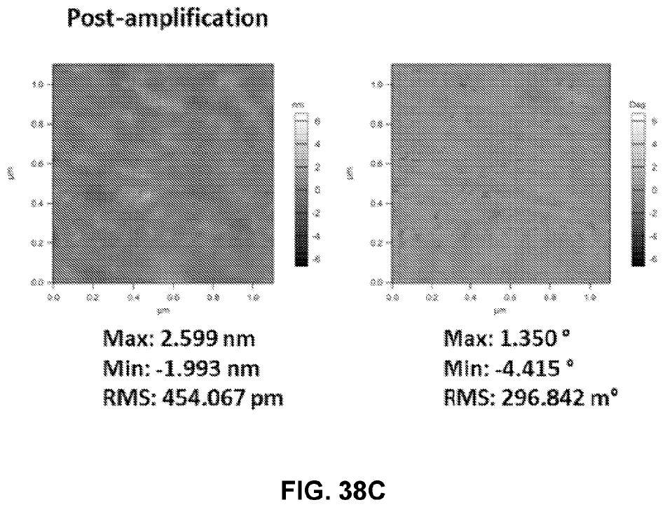

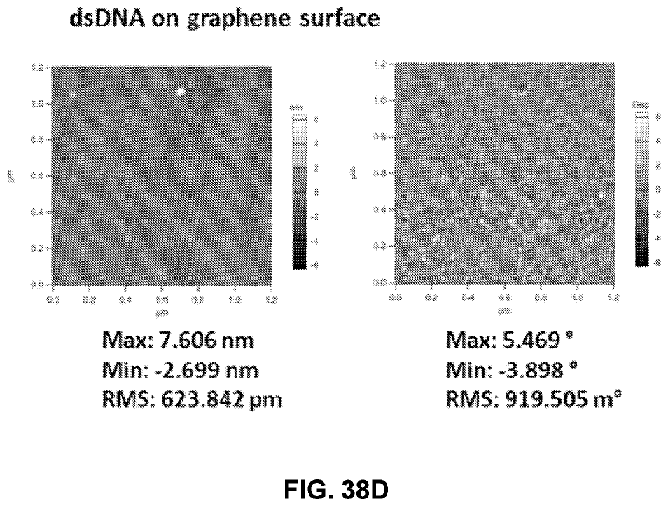

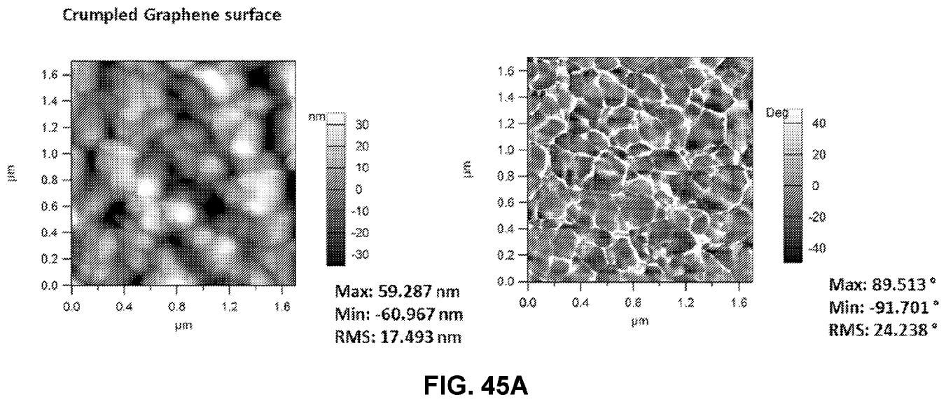

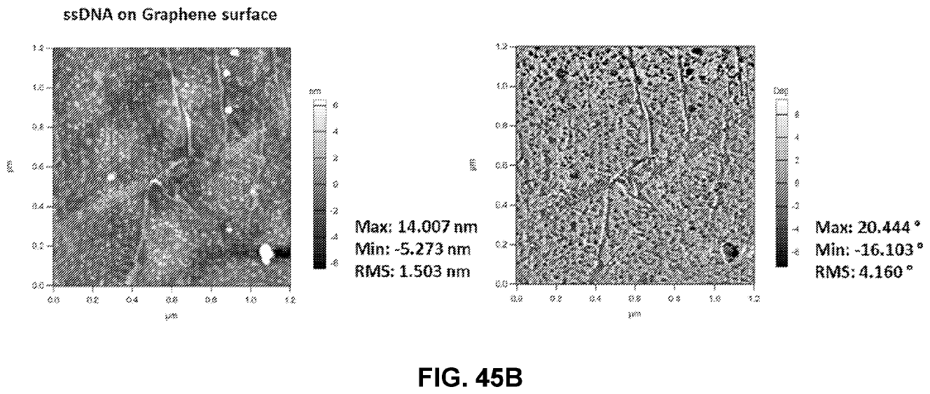

Our crumpled graphene FET biosensor fabrication process was adapted from previously published protocol³². Briefly, a 2 mm×14 mm graphene channel was transferred onto a thermoplastic polystyrene substrate and annealed at 110° C. for 4 hours. During the annealing process, the underlying pre-strained thermoplastic substrate shrinks and results in the buckling and crumpling of graphene channel. For the flat graphene FET used in this study, this annealing step was omitted. Finally, the source and drain metal electrodes are formed and an ionic solution reservoir was created around the graphene channel and gate voltage is applied directly to the top of the ionic solution placed in the reservoir in the device. The black regions on FIG. 37A show the source and drain contacts, and graphene is visible as a shaded rectangular channel between source and drain contacts surrounded by solution reservoir. The morphology of the crumpled graphene surface shown by AFM image in on FIG. 37B depict disorganized structures with fine wrinkles as small as a few hundred nanometers. Theoretical Debye length modulation in our crumpled gFET sensors can be expected to arise from these small concave wrinkles. The AFM topography data for crumpled graphene also shows increased surface roughness (RMS˜17.49 nm) compared to flat graphene surface (RMS˜0.65 nm) (FIG. 45A-45B). It's important to note that these estimated roughness values are often underestimated and are limited in resolution by AFM tip dimensions. To validate the electrical properties of crumpled graphene, source-drain current was measured and found to be between 4-8 kΩ.

FIG. 37C shows the results of electrical sensing of ssDNA in 1× PBS for both flat and crumpled graphene. For the electrical sensing, changes in source-drain current and the Dirac voltage point shifts were measured due to physisorption of ssDNA molecules on our sensor. For ssDNA measurements, we chose FIP primer sequence (Table 1), and tested the sequential increase in concentrations of ssDNA from 2 aM to 2 uM on the same device. More details about the measurement protocol is presented in the methods section. For flat graphene device, there was no significant Dirac point shift below 20 fM, with an overall negative Dirac point shift of 10 mV from 2 aM to 200 pM. From 200 pM to 2 uM, anomalous positive Dirac point shifts of up to 26 mV were observed, possibly related to device stability issues as these tests were sequentially performed on the same device. We observed high device-to-device variability in flat graphene measurements resulting in larger error bars (std. dev for n=3) as compared to that of crumpled graphene measurements. For crumpled graphene devices we measured an overall shift of ˜36 mV from 2 aM to 2 uM samples with shift of ˜6 mV even for the lowest ssDNA concentration of 2 aM compared to the negative control (PBS only). Due to the above differences in device performance, we focused on crumpled graphene FET sensors for the remaining experiments in our study.

Next, we investigated the physical adsorption of dsDNA on crumpled graphene FET biosensors by using a similar protocol as above and FIG. 2d shows the electrical measurements. We added synthetic dsDNA (Table 3) at concentrations from 2 aM to 2 uM and measured the Dirac point shifts for each condition as mentioned in the methods sections. In contrast to the ssDNA experiments, the overall shift from 2 aM to 2 uM of dsDNA tested sequentially on the same device was only ˜6 mV, with shift for 2uM dsDNA itself being negligible. This confirms that dsDNA do not strongly adsorb on the crumpled graphene FET sensors and hence, we do not expect a significant contribution from dsDNA in our amplification experiments.

AFM characterization of physical adsorption of molecules on graphene surface: Structural features of the flat graphene surface with physiosorbed molecules were characterized using an atomic force microscope (AFM) for different test cases and the results are shown in FIGS. 38A-38D. As seen in the AFM images in FIG. 38A, the topography of a bare graphene surface is mostly flat (RMS roughness˜0.6 nm) with some defects. When physisorption of ssDNA molecules was tested by incubation for 15 minutes, followed by PBS and DI water rinsing, drying and imaging in air, an increase in the surface roughness (RMS˜1.5 nm) and morphology of the flat graphene was observed (FIGS. 45A-45B). Since our platform differentiates amplified from non-amplified samples by detecting the reduction in primer concentrations and associated Dirac voltage shifts, we characterized graphene surfaces challenged with pre-amplification (time 0) and post-amplification (time 60) solutions diluted in PBS 1×. The solution dilution was the same as the ones used for electrical measurements in FIGS. 37A-37D. For all AFM physisorption tests, same protocol as above for ssDNA was used and more details are mentioned in the methods section. For pre-amplified test case shown in FIG. 38B, an increase in the surface roughness and morphology of the flat graphene was observed similar to ssDNA tests, with RMS values of ˜1.5 nm, which was 2.4 times higher than the flat surface. The phase image confirms these features with the appearance of distinct black structures on the flat graphene surface. In contrast, when pre-amplified test case was tested for physisorption (FIG. 38C), surface roughness of RMS˜0.45 nm was observed, which is similar to that of flat graphene. Phase image also shows smoother surface without black features. It must be taken into consideration that target dsDNA is in lower concentrations than ssDNA (primers) at time 0 of the amplification reaction. In the LAMP amplification reaction, primer consumption in forming replicates of target dsDNA causes the ssDNA concentration in the reaction to decrease. We can see in FIG. 38C that high concentration of dsDNA (amplified product) as well as residual amplification molecules dried onto the graphene surface does not significantly change the graphene surface roughness. This was further confirmed in FIG. 38D in which 100nM of synthetic dsDNA (sequence given in Table 3) tested for physisorption on the graphene surface showed a similar roughness to that seen in FIGS. 38A, 38C. Note that we did not perform the adsorption studies on crumpled graphene as the AFM tip is not able to resolve differences with and without DNA adsorption on the crumpled graphene surface.

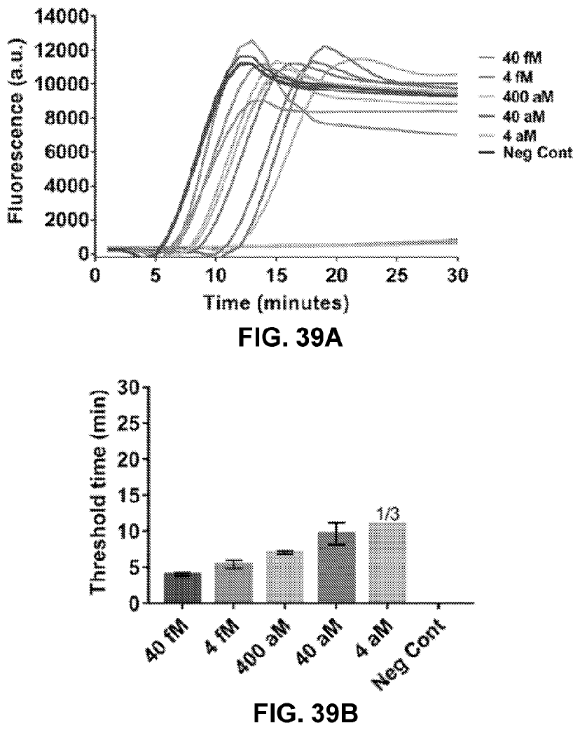

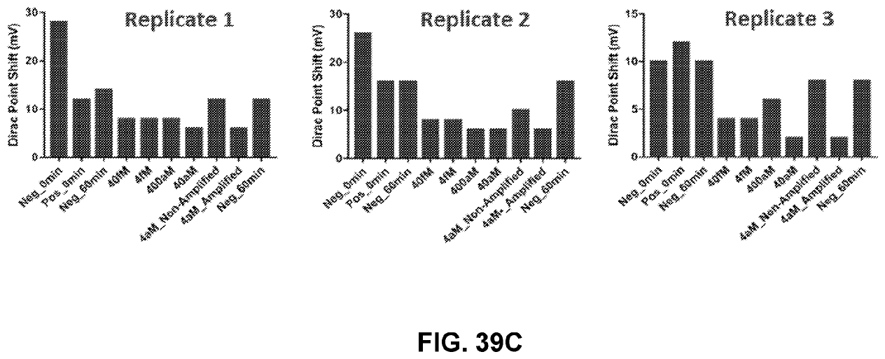

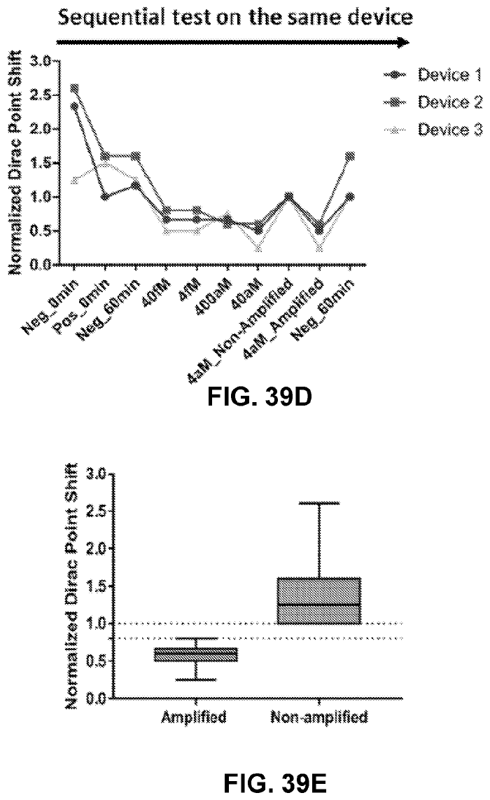

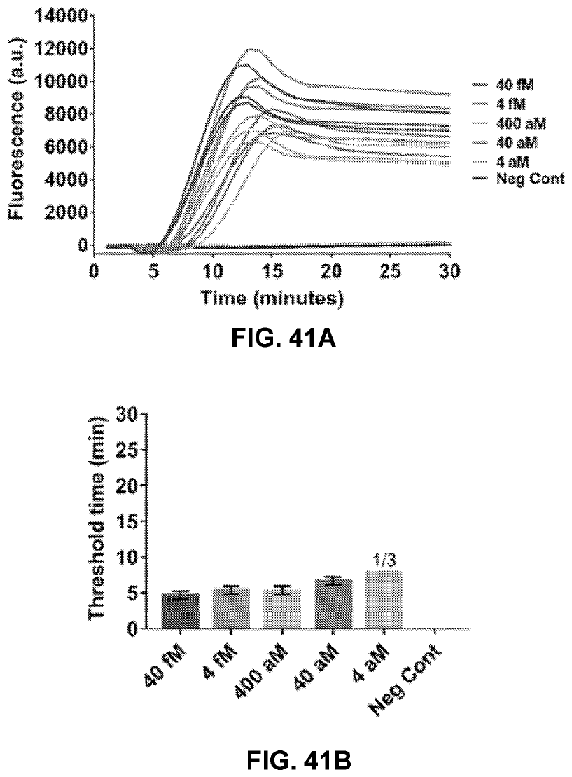

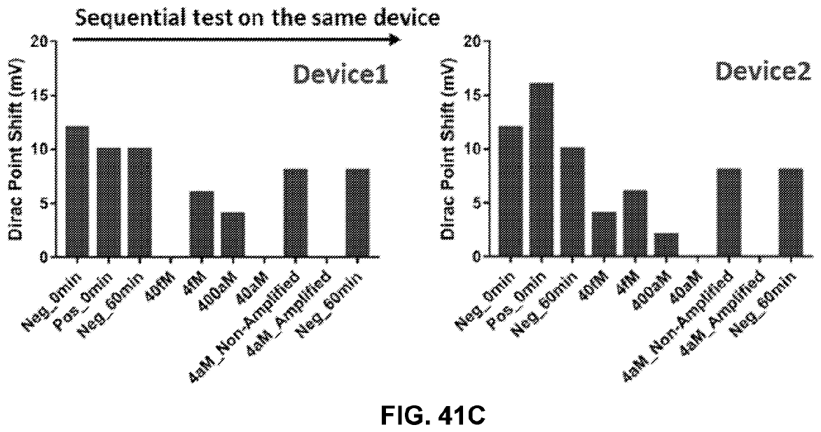

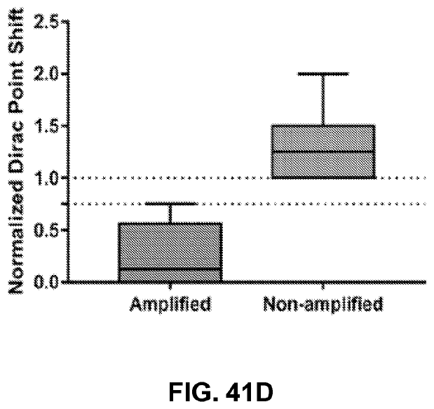

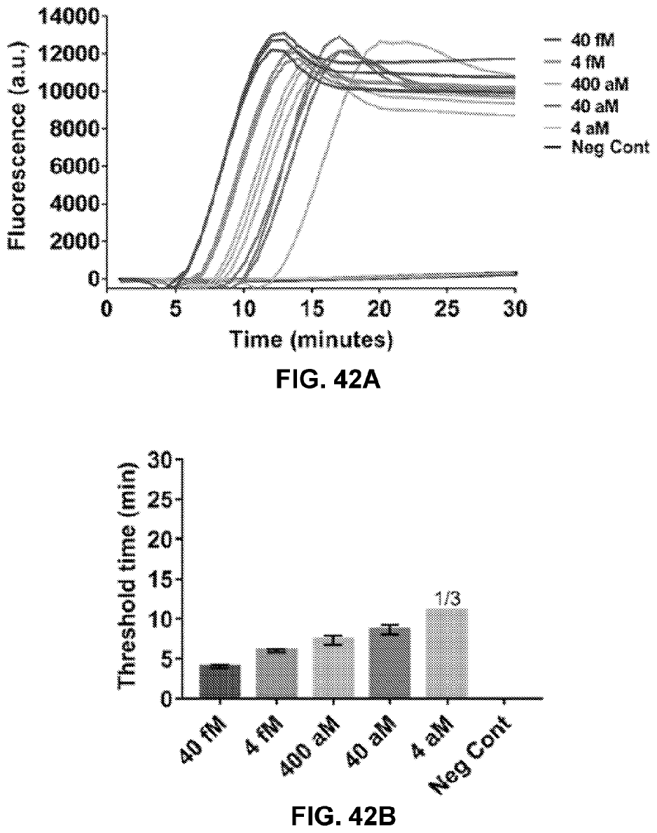

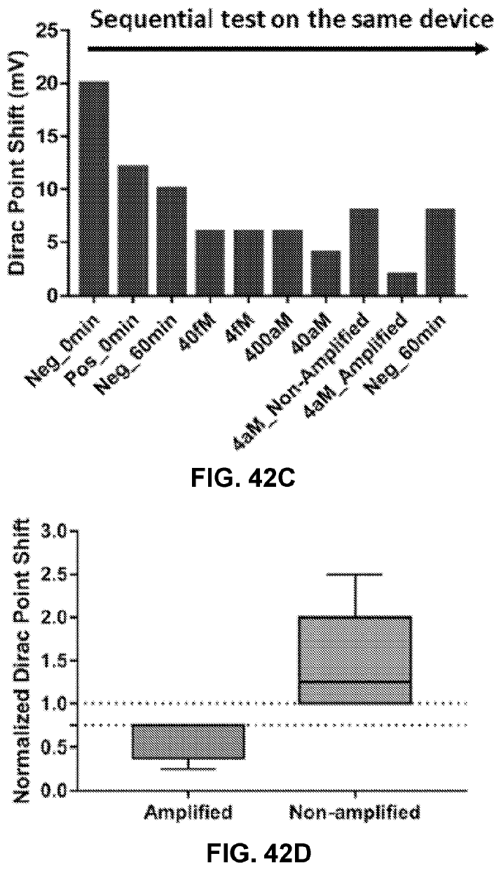

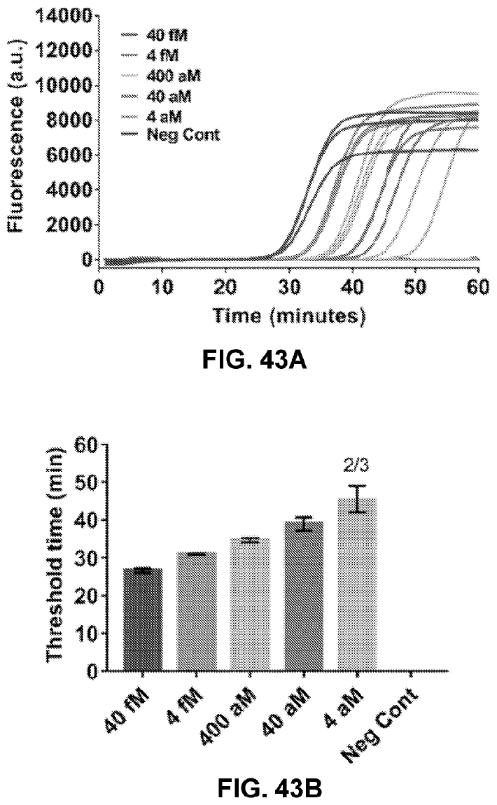

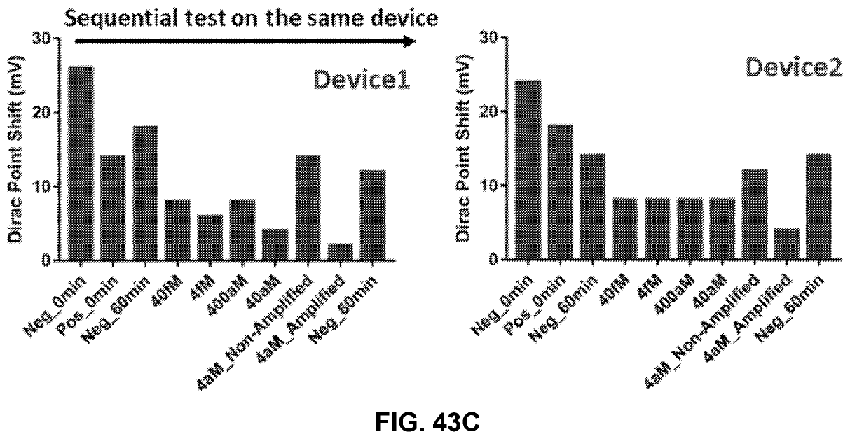

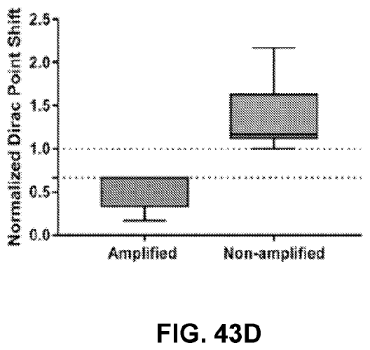

Attomolar E. coli DNA detection using crumpled gFET biosensors: To test the ability of our crumpled gFET biosensor to distinguish positive amplification as compared to LAMP reactions where no target was present, we first performed real-time LAMP reactions for E. coli DNA with Evagreen dye to confirm that DNA amplification has occurred. FIGS. 39A and 39B show the fluorescence measurements and threshold times for amplification of 4 aM to 40 fM E. coli genomic DNA using LAMP amplification reaction. For detection of E. coli DNA, primers (ssDNA) complementary to the eae gene was used to identify and begin the amplification reaction (see Table 3 for sequences). Amplification of DNA concentrations from 40 fM to 40 aM can be seen for each replicate; however, only 1 out of 3 replicates for 4 aM DNA concentration resulted in amplification. 4 aM translates to ˜2.4 copies per microliter of starting sample and at these extremely low concentrations, the sampling of the sample can result in errors. This thermocycler data will serve as a control for the following electrical measurements on these same samples post amplification. These amplified and non-amplified samples (4 aM, negative control) were diluted 1:100 in 1× PBS and sequentially tested on 3 separate crumpled gFET devices to measure the Dirac Point shift for each case. The results are shown in FIG. 39C. On each crumpled gFET sensor, we first measured the negative control sample before the beginning of the LAMP reaction (time =0 min), after which the 40 fM sample at time=0 min was measured for the Dirac Point shift. After 60 min of LAMP reaction, the samples were measured again starting with the negative control and each of the target DNA positive concentrations from 40 fM to 4 aM. Finally, we measured the negative control sample from time=60 min again to confirm that the graphene surface was not saturated with DNA during the measurement. In FIG. 39C, we can see that for each replicate the difference between amplified and non-amplified samples was greater than or equal to 4 mV showing the platform's ability to clearly distinguish the positive samples from the negative samples. The non-amplified 4 aM samples served as blind tests and confirmed that our platform can distinguish these from the amplified 4 aM samples. For amplified samples, we see lower Dirac point shifts compared to non-amplified samples, since the primers (ssDNA) are converted into dsDNA during the amplification and dsDNA do not produce significant shift in Dirac point as shown previously in FIG. 37D. The data from each replicate was then normalized to its lowest negative control Dirac voltage shift measurement and plotted in FIG. 39D. All the normalized data was then clustered into “Amplified” and “Non-amplified” samples and a clear distinction between the two clusters can be seen in FIG. 39E. This shows that negative samples can be clearly distinguished from positive samples with a detection limit of up to 4 aM. FIGS. 41A-42D show similar trends but with slightly lower average Dirac voltage shifts for 1× primer and 1:100,000 and 1:10,000 dilution in 1× PBS, respectively. FIGS. 43A-43D shows the repeat of FIGS. 39A-39E but with a reduced 0.1× primer concentration. Reduced primer concentration did not affect the electrical measurements but delayed the amplification threshold times by ˜20 minutes compared to 1× primer concentration, proving that a lower primer concentration results in delayed amplification and that are reactions are indeed primer concentration limited.

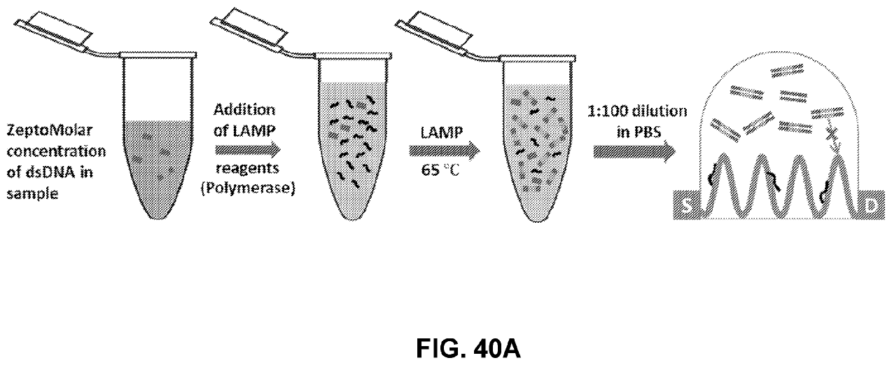

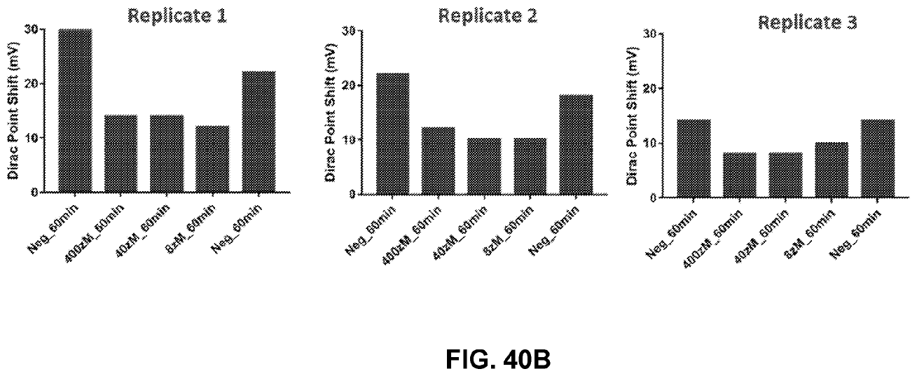

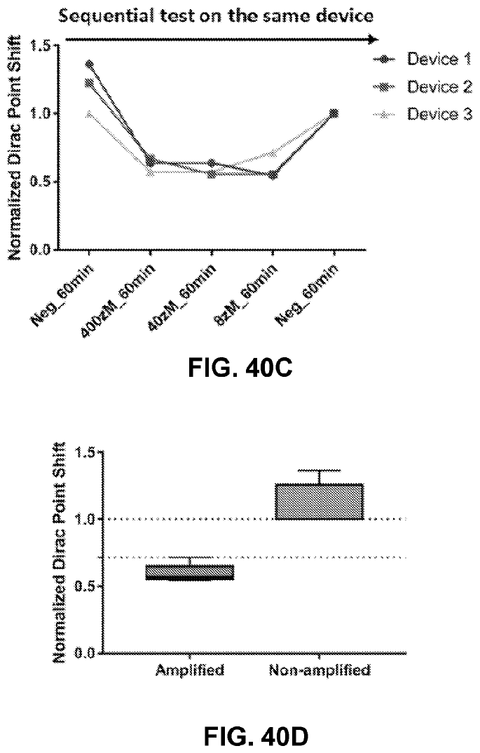

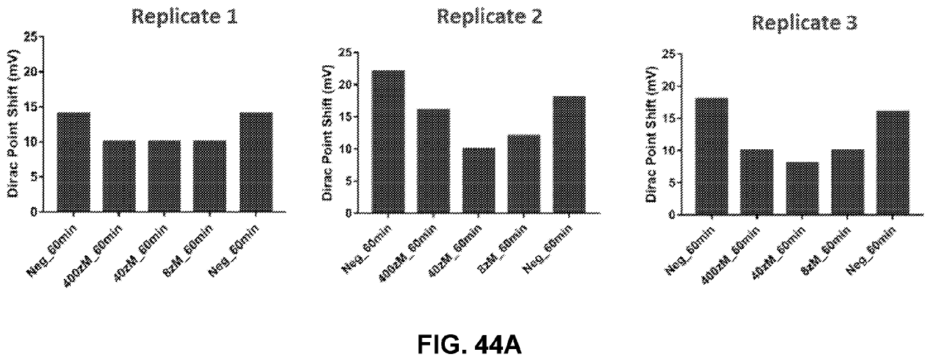

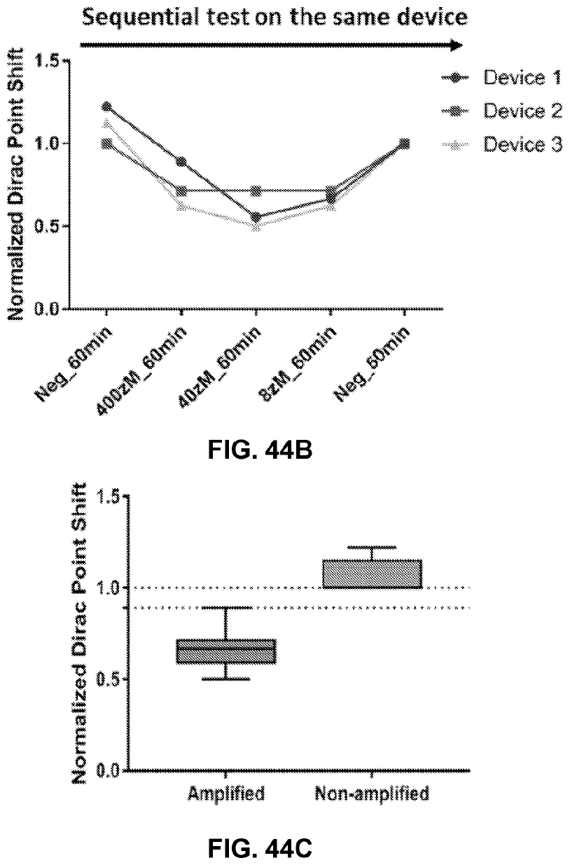

Zeptomolar E. coli DNA detection using crumpled gFET biosensors: In FIG. 40A, we show a schematic of a Zeptomolar LAMP reaction in which ˜3 target E. coli dsDNA copies were spiked in 572 uL of water as the starting sample, resulting in an 8 zM concentration. LAMP reagents including the Bst polymerase were thereafter added for the reaction to occur at 65 C for 60 min. Similarly, higher concentrations of E. coli dsDNA (40 and 400 zM) were spiked and amplified in LAMP reactions. The products of each reaction were mixed and diluted 1:100 in 1× PBS and then electrically measured on the FET device. Sequentially testing on the same device, we first measured the negative control sample post LAMP reaction (time=60 min). Following that, we measured the 400, 40, and 8 zM concentration samples reactions for Dirac Point Shift with PBS rinses in between. Finally, we measured the negative control sample again in order to confirm that the graphene surface was not saturated with DNA during the measurement. In FIG. 40B, 3 replicates of this experiment showed similar results. The negative control measurement showed a Dirac Point Shift of up to 30 mV. Differences in Dirac Point Shifts between replicates can be attributed to the variability in resistance of each device, which could be due to variability in the fabrication process. Nevertheless, the Dirac point shift for detection of amplification in all positive reactions (8 zM to 400 zM) ranged from 8 mV to 14 mV. A difference of 4 mV to 18 mV was measured for Dirac Shift between the 8 zM sample and Negative Control in these three replicates. The data from each replicate was then normalized to its lowest negative control Dirac voltage shift measurement and plotted in FIG. 40C. FIG. 40D shows all the normalized data clustered into “Amplified” and “Non-amplified” samples and a clear distinction between the two clusters can be seen. This shows that negative samples can be clearly distinguished from positive samples with a detection limit of 8 zM. FIGS. 44A-44C shows the repeat of FIGS. 40A-40D but with a reduced 0.1× primer concentration. Reduced primer concentration did not affect the electrical measurements and, amplified versus non-amplified samples could be distinguished clearly with no overlap.

We demonstrate detection of enzymatic DNA amplification using crumpled graphene FET with detection limits down to Zeptomolar target concentrations in the starting sample. Our platform deploys the evolutionary sensitivity and robustness of Bst polymerase combined with target specific primers, while using crumpled graphene FET to electrically detect the amplification by sensing the reduction in the primer molecules. We also characterize the physisorption of ssDNA, dsDNA, and LAMP reaction mix, pre- and post-amplification, to show lower physisorption of molecules (lower roughness in AFM) post-amplification due to reduced or consumed primers. Crumpled graphene FET provided better and higher sensitivity and Dirac voltage shifts in comparison to the flat counterpart and hence were chosen for our study. For our enzymatic reactions, we chose Loop Mediated Isothermal

Amplification (LAMP) as it only needs a constant temperature for performing reactions and will allow easy translation into point-of-care and small footprint devices in the future. Moreover, since Bst polymerase has been applied to direct detection from complex matrices such as blood or saliva and requires specificity of 6 unique target specific primers, our platform will be more suitable for direct processing of complex samples in the future and will be superior to direct hybridization based approaches which rely on a single sequence/primer specificity. Compared to the conventional silicon based ISFET based approaches which sense changes in pH, require low buffer capacity reaction, and have a reported a detection limit of >10 aM, our platform can sense target down to zeptomolar concentrations with electrical sensing possible directly in 1× PBS. Due to these reasons, the platforms provided herein allow translation of complex lab-based diagnostic and genotyping amplification assays to truly point of care and bedside platforms.

References for Example 2:

1. Sinha, M. et al. Emerging technologies for molecular diagnosis of sepsis. Clinical Microbiology Reviews 31, (2018).

2. Peker, N., Couto, N., Sinha, B. & Rossen, J. W. Diagnosis of bloodstream infections from positive blood cultures and directly from blood samples: recent developments in molecular approaches. Clinical Microbiology and Infection 24, 944-955 (2018).

3. Star, A. et al. Label-free detection of DNA hybridization using carbon nanotube network field-effect transistors. Proc. Natl. Acad. Sci. United States Am. 103, 921-926 (2006).

4. Dong, X. et al. Label-Free Electronic Detection of DNA Using Simple Double-Walled Carbon Nanotube Resistors. J. Phys. Chem. C 112, 9891-9895 (2008).

5. Zhang, G. J. et al. DNA sensing by silicon nanowire: Charge layer distance dependence. Nano Lett. 8, 1066-1070 (2008).

6. Dong, X. et al. Electrical Detection of Femtomolar DNA via Gold-Nanoparticle Enhancement in Carbon-Nanotube-Network Field-Effect Transistors. Adv. Mater. 20, 2389-2393 (2008).

7. Liu, Y. et al. Detection of 12 Common Food-Borne Bacterial Pathogens by TaqMan Real-Time PCR Using a Single Set of Reaction Conditions. Front. Microbiol. 10, (2019).

8. Itahashi, M., Higaki, S., Fukuda, M. & Shimomura, Y. Detection and quantification of pathogenic bacteria and fungi using real-time polymerase chain reaction by cycling probe in patients with corneal ulcer. Arch. Ophthalmol. 128, 535-540 (2010).

9. Xu, G., Abbott, J. & Ham, D. Optimization of CMOS-ISFET-based biomolecular sensing: Analysis and demonstration in DNA detection. IEEE Trans. Electron Devices 63, 3249-3256 (2016).

10. Toumazou, C. et al. Simultaneous DNA amplification and detection using a pH-sensing semiconductor system. Nat. Methods 10, 641-646 (2013).

11. Duarte-Guevara, C. et al. On-chip electrical detection of parallel loop-mediated isothermal amplification with DG-BioFETs for the detection of foodborne bacterial pathogens. RSC Adv. 6, 103872-103887 (2016).

12. Graphene Field Effect Transistors for Biomedical Applications: Current Status and Future Prospects. Diagnostics 7, 45 (2017).

13. Rasheed, P. A. & Sandhyarani, N. Graphene-DNA electrochemical sensor for the sensitive detection of BRCA1 gene. Sensors Actuators, B Chem. 204, 777-782 (2014).

14. Ping, J., Vishnubhotla, R., Vrudhula, A. & Johnson, A. T. C. Scalable Production of High-Sensitivity, Label-Free DNA Biosensors Based on Back-Gated Graphene Field Effect Transistors. ACS Nano 10, 8700-8704 (2016).

15. Xu, G. et al. Electrophoretic and field-effect graphene for all-electrical DNA array technology. Nat. Commun. 5, (2014).

16. Hajian, R. et al. Detection of unamplified target genes via CRISPR-Cas9 immobilized on a graphene field-effect transistor. Nat. Biomed. Eng. 3, 427-437 (2019).

17. Hwang, M. T. et al. DNA Nanotweezers and Graphene Transistor Enable Label-Free Genotyping. Adv. Mater. 30, 1802440 (2018).

18. Hwang, M. T. et al. Highly specific SNP detection using 2D graphene electronics and DNA strand displacement. Proc. Natl. Acad. Sci. 113, 7088-7093 (2016).

19. Campos, R. et al. Attomolar Label-Free Detection of DNA Hybridization with Electrolyte-Gated Graphene Field-Effect Transistors. ACS Sensors 4, 286-293 (2019).

20. Justino, C. I. L., Gomes, A. R., Freitas, A. C., Duarte, A. C. & Rocha-Santos, T. A. P. Graphene based sensors and biosensors. TrAC - Trends in Analytical Chemistry 91, 53-66 (2017).

21. Liu, Y., Dong, X. & Chen, P. Biological and chemical sensors based on graphene materials. Chem. Soc. Rev. 41, 2283-2307 (2012).

22. Yang, W. et al. Carbon Nanomaterials in Biosensors: Should You Use Nanotubes or Graphene? Angew. Chemie Int. Ed. 49, 2114-2138 (2010).

23. Geim, A. K. & Novoselov, K. S. The rise of graphene. Nat. Mater. 6, 183-191 (2007)

24. Mao, S., Chang, J., Zhou, G. & Chen, J. Nanomaterial-enabled Rapid Detection of Water Contaminants. Small 11, 5336-5359 (2015).

25. Mao, S., Lu, G. & Chen, J. Nanocarbon-based gas sensors: Progress and challenges. J. Mater. Chem. A 2, 5573-5579 (2014).

26. Mao, S. Graphene Field-Effect Transistor Sensors. in Graphene Bioelectronics 113-132 (Elsevier Inc., 2018). doi:10.1016/B978-0-12-813349-1.00005-6

27. Zhan, B. et al. Graphene Field-Effect Transistor and Its Application for Electronic Sensing. Small n/a-n/a (2014). doi:10.1002/sm11.201400463

28. Chen, T. Y. et al. Label-free detection of DNA hybridization using transistors based on CVD grown graphene. Biosens. Bioelectron. 41, 103-109 (2013).

29. Cai, B. et al. Ultrasensitive label-free detection of PNA-DNA hybridization by reduced graphene oxide field-effect transistor biosensor. ACS Nano 8, 2632-2638 (2014).

30. Zheng, C. et al. Fabrication of Ultrasensitive Field-Effect Transistor DNA Biosensors by a Directional Transfer Technique Based on CVD-Grown Graphene. ACS Appl. Mater. Interfaces 7, 16953-16959 (2015).

31. Shoorideh, K. & Chui, C. 0. On the origin of enhanced sensitivity in nanoscale FET-based biosensors. Proc. Natl. Acad. Sci. U. S. A. 111, 5111-5116 (2014).

32. Wang, M. C. et al. Heterogeneous, Three-Dimensional Texturing of Graphene. Nano Lett. 15, 1829-1835 (2015).

33. Kang, P., Wang, M. C., Knapp, P. M. & Nam, S. Crumpled Graphene Photodetector with Enhanced, Strain-Tunable, and Wavelength-Selective Photoresponsivity. Adv. Mater. 28, 4639-4645 (2016).

34. Leem, J., Wang, M. C., Kang, P. & Nam, S. Mechanically Self-Assembled, Three-Dimensional Graphene-Gold Hybrid Nanostructures for Advanced Nanoplasmonic Sensors. Nano Lett. 15, 7684-7690 (2015).

35. Hwang, M. T. et al. Ultrasensitive detection of nucleic acids using deformed graphene channel field effect biosensors. Nat. Commun (UNDER Rev.

36. Hwang, M. T. et al. Ultrasensitive detection of nucleic acids using deformed graphene channel field effect biosensors. Nat. Commun. 11, 1543 (2020).

37. Li, M. et al. Pt/single-stranded DNA/graphene nanocomposite with improved catalytic activity and CO tolerance. J. Mater. Chem. A 3, 10353-10359 (2015).

38. Wei, G., Li, Q., Steckbeck, S. & Ciacchi, L. C. Direct force measurements on peeling heteropolymer ssDNA from a graphite surface using single-molecule force spectroscopy. Phys. Chem. Chem. Phys. 16, 3995-4001 (2014).

39. Banerjee, S. et al. Slowing DNA transport using graphene-DNA interactions. Adv. Funct. Mater. 25, 936-946 (2015).

40. Huang, P.-J. & Liu, J. Separation of Short Single- and Double-Stranded DNA Based on Their Adsorption Kinetics Difference on Graphene Oxide. Nanomaterials 3, 221-228 (2013).

41. Jansson, L. & Hedman, J. Challenging the proposed causes of the PCR plateau phase. Biomol. Detect. Quantif. 17, (2019).

42. Notomi, T. Loop-mediated isothermal amplification. Nippon rinsho. Japanese journal of clinical medicine 65, 957-961 (2007).

43. Ganguli, A. et al. Hands-free smartphone-based diagnostics for simultaneous detection of Zika , Chikungunya , and Dengue at point-of-care. 1-13 (2017). doi:10.1007/s10544-017-0209-9

44. Notomi, T. et al. Loop-mediated isothermal amplification of DNA. Nucleic Acids Res. 28, E63 (2000).

45. Wang, F., Jiang, L. & Ge, B. Loop-mediated isothermal amplification assays for detecting Shiga toxin-producing Escherichia coli in ground beef and human stools. J. Clin. Microbiol. 50, 91-97 (2012).

Materials and Methods:

Fabrication of Graphene FET: Graphene grown on copper film substrate was obtained from Grolltex and graphenesquare. Poly(methyl methacrylate) (PMMA) was obtained from Microchem. The high-quality graphene film on a grown copper film substrate was transferred and attached to a glass slide with double-sided adhesive. The top side of the graphene was spin-coated at 2000 rpm for 1 min, with a PMMA layer for protection from downstream copper etching as well as support for the graphene layer post etching. After, the spin coated PMMA/graphene sample was removed from the glass slide and the back side of the graphene was removed by oxygen plasma etching (at 100W for 13 min). Post etching, the sample was cut into 2 mm×14 mm pieces. Then, PMMA/graphene layer was delaminated from the copper film substrate using 0.1 M sodium persulfate (copper etchant) (Sigma Aldrich) for about 5 hours. The etched sample was incubated in deionized (DI) water overnight. The PMMA/graphene sample was then transferred onto a polystyrene sheet, after which the PMMA layer was removed by acetic acid for 10 min. The graphene on polystyrene sample was finally rinsed with DI water and dried with compressed air.

To fabricate the transistor, conducting silver paste (Ted Pella, Inc.) was used as source and drain electrodes at the two ends of the graphene on polystyrene. Silicone rubber (Dow Corning) was used to insulate source and drain electrodes from liquid and construct a solution reservoir for the LAMP sample or PBS. Before using for electrical measurement, the FET device is incubated with 1× PBS overnight.

DNA and Bacteria: Genomic DNA of Escherichia coli (O157:H7), NR-4629, was obtained through BEI Resources. These genomic DNA vials were aliquoted and stored at −80 C. Appropriate stock volumes were used either for diluted to the right concentration in buffer or water.

Primer Sequences

All primer sequences for the LAMP reactions were synthesized by Integrated DNA Technology (IDT). Primer sequences for E. coli eae gene were obtained from Wang et al⁴⁵. Single-stranded DNA samples used in control measurements were the FIP primers (37 bases). The double stranded DNA sample contains complementary strands. See for the sequences of primers and DNA strands.

LAMP Reactions: LAMP assays were designed to target the eae gene for E. coli. The LAMP assay consisted of the following components: 1× final concentration of the isothermal amplification buffer (New England Biolabs), 1.025 mmol L⁻¹ each of deoxy-ribonucleoside triphosphates (dNTPs), 4 mmol L⁻¹ of MgSO4 (New England Biolabs), and 0.29 mol L⁻¹ of Betaine (Sigma-Aldrich). These individual components were stored according to manufacturer instructions and a mix including all components was created fresh prior to each reaction. In addition to the buffer components, 0.47 U μL⁻¹ Bst 2.0 WarmStart

DNA Polymerase (New England Biolabs), 1 mg/ml BSA (New England Biolabs), and 0.74× EvaGreen (Biotium), a double-stranded DNA (dsDNA) intercalating dye, was included in the reaction. 1× primer concentration in each reaction consisted of 0.15 μM of F3 and B3, 1.17 μM FIP and BIP, and 0.59 μM of LoopF and LoopB primers. 0.1× primer concentration in each reaction consisted of 0.015 μM of F3 and B3, 0.117 μM FIP and BIP, and 0.059 μM of LoopF and LoopB primers. 1 uL of template in water was added to make the final reaction volume 16 uL. For negative assays, water was substituted for the template. Ten-fold serial dilutions in water of the template were done as necessary for the LAMP assays.

All the LAMP reactions were carried out in 0.2 mL PCR reaction tubes in an Eppendorf Mastercycler® realplex Real-Time PCR System. The tubes were incubated at 65° C. for 60 min in the thermocycler, and fluorescent data were recorded every 1 min. Fluorescence data were recorded after each cycle of the reaction. Three repeats were done for each reaction.

Amplified products for each concentration were pooled together for electrical measurements. Unamplified products for negative control were also pooled together for electrical measurements.

The Zeptomolar reactions were designed for starting sample DNA concentrations of 8 zM, 40 zM, and 400 ZM. For 8 zM sample concentration of template DNA, 1.14 uL of DNA (2.91 copies) was added to 571.98 uL of water. For 40 zM sample concentration of DNA, 1.43 uL of DNA (3.63 copies) was added to 143 uL of water. For 400 zM sample concentration of DNA, 14.3 uL of DNA (36.3 copies) was added to 143 uL of water. After these starting samples were prepared, the LAMP assay at concentrations mentioned above were added to make the total reaction volume 1000 uL (for 8 zM reaction) or 250 uL (for 40 and 400 zM reactions). The reactions were carried out in 1.5 mL Eppendorf tubes in a digital heater at 65° C. for 60 min (Thermo Fisher Scientific).

Amplification Data Analysis: The off-chip raw fluorescence curves and amplification threshold bar graphs were analyzed using a MATLAB script and plotted using GraphPad Prism. The threshold time for each curve was taken as the time required for each curve to reach 10% of the total intensity. The amplification threshold bar graphs are show a mean of 3 samples.

Electrical Measurements and Analysis: I-V curves of gFET were measured in a semiconductor parameter analyzer equipped with a probe station (Company). Post overnight incubation with PBS (Thermo Fisher Scientific), the device was rinsed 3 times with 1× PBS, and finally, 50 uL of 1× PBS was added for measurement. Silver wire was used as an electrode, which applied gate voltage (Vg) to the PBS buffer solution. Vg was swept from 0 to 1 V in steps of 2 mV and drain—source voltage (Vds) was adjusted between 0.03 and 0.1 V depending on the resistance of the device. Drain-source current (Ids) for all sample measurements on the specific device was obtained at a constant Vds. Sweeping the Vg, Ids measurements were collected, and the Dirac voltage was found. The Dirac voltage is the minimum point in the resulting I-V curve of the gFET device.

Before measuring the LAMP samples, measurements of PBS solution were consistently done every 1 hr to find a stable Dirac voltage.

Measurement of DNA Samples: Before measuring the amplified and unamplified DNA samples from the LAMP reactions, each sample was diluted with 1× PBS (ranging from 1:100 to 1:1E5 dilution). In the cases for ssDNA and dsDNA adsorption tests, these DNA sample concentrations were luM in PBS.

Before measuring the amplified DNA samples, negative and positive control (pre-LAMP reaction) samples were electrically measured. Negative control sample was added on the device and incubated for 15 min, after which the device was rinsed three times with 1× PBS. After, 50 uL of 1× PBS was added on the device and sequential electrical measurements were conducted. The final measurement at which no changes in Dirac voltage was seen was taken as the final Dirac point, using which the Dirac Point Shift was calculated. The difference in Dirac voltage (or Dirac Point Shift) between the average and the Dirac voltage before addition of the sample was calculated. Thereafter, positive controls and amplified DNA samples were measured in the same protocol, and Dirac point shift due to physical adsorption of each sample was calculated from PBS to PBS measurements. After amplified DNA samples were measured, negative control samples were again measured to confirm that there is no change in the Dirac voltage.

Atomic Force Microscopy (AFM) for DNA and Graphene surface visualization: AFM images were acquired for all sample types to visualize the graphene surface morphology due to adsorbed biomolecules. All AFM images were acquired using a tapping mode Asylum Research MFP-3D AFM. Silicon cantilevers with a spring constant of 42 N/m (PPP-NCHR; Nanosensor) were used for imaging in air. The samples were incubated 15 min on the flat graphene, then rinsed with 1× PBS three times and DI water. The samples were dried using low pressure N2 gas. The MFP-3D AFM system software was used for analyzing image data. Max and min in the figures indicate the maximum and minimum height measured on the graphene surface, respectively. RMS, is the root mean square roughness of the graphene surface.

TABLE 3
(from Example 2):

| SEQ | | |
|---|---|---|
| ID | Primer/ | |
| No: | Probe name | Sequence (5′-3′) |
| 10 | F3 | TGACTAAAATGTCCCCGG |
| 11 | B3 | CGTTCCATAATGTTGTAACCAG |
| 12 | FIP | GAAGCTGGCTACCGAGACTCCCAAAAGCAACATGACCGA |
| 13 | BIP | GCGATCTCTGAACGGCGATTCCTGCAACTGTGACGAAG |
| 14 | Loop F (LF) | GCCGCATAATTTAATGCCTTGTCA |
| 15 | Loop B (LB) | ACGCGAAAGATACCGCTCT |
| 16 | ssDNA | GAAGCTGGCTACCGAGACTCCCAAAAGCAACATGACCGA |
| 17 | dsDNA | CGC CAA GCT TTT TAA CAG TGG CCT TAT TAA ATG ACT |
| | | TCT CTA AAG GAT CCC GGG TAC CGA |

### EXAMPLE 3

### Ultrasensitive Detection of Dopamine, IL-6 and SARS=CoV-2 Proteins with Crumpled Graphene FET Biosensors

This example illustrates use of the devices and methods described herein to detect a variety of biomarkers.

Universal platforms for biomolecular analysis using label free sensing modalities can address a range of important diagnostic challenges in infectious disease, cancer, and other important areas. Electrical field effect sensors are an important class of devices that can enable point of care sensing by probing the intrinsic charge in biological entities. Use of graphene and especially curved or crumpled graphene for this application is especially promising. We have previously reported the lowest limit of detection (LoD) on electrical label-free field effect-based sensors using single or double stranded DNA molecules on the crumpled graphene FET platform. Here, we report field effect transistor-based biosensing of other important biomarkers including small molecules and proteins. We systematically evaluated and optimized the performance of these devices by studying the effect of the crumpling ratio on electrical double layer (EDL) formation and bandgap opening on the graphene. We show that a small and electroneutral molecule dopamine can be captured by an aptamer and the conformation change of the probe molecule induced electrical signal changes in the sensor. Three different kinds of proteins were captured with specific antibodies including interleukin-6 (IL-6) and viral proteins. All tested biomarkers were detectable with the highest sensitivity reported on a label-free electrical platform. Significantly, two COVID-19 related proteins, nucleocapsid (N-) and spike (S-) proteins antigens were successfully detected in PBS with extremely low LoDs. This label-free electrical antigen tests addresses the challenge of rapid, point of care diagnostics for infectious disease and other important clinical conditions.

An all electrical biosensor is of great interest, especially in the pandemic situation as these devices can be used for clinical diagnosis, point-of-care testing, and on-site detection^{[1]}. The COVID-19 pandemic, amongst other healthcare challenges, has pointed attention to the importance of rapid and effective diagnosis of diseases^{[2]}.

FET-based biosensors allow label-free and highly sensitive biomolecular detection on integrated lab-on-a-chip systems. Detection of pH, nucleic acids, proteins and other biomarkers have been reported using FET biosensor with many different channel materials such as conventional silicon, nanowire and 2D materials. Among those, graphene is an attractive material due to its unique mechanical, chemical and electrical properties. CVD-grown graphene exhibits superior sensitivity in charge-based detection of biomolecules owing to its single-atom thickness and ultimate aspect ratio.

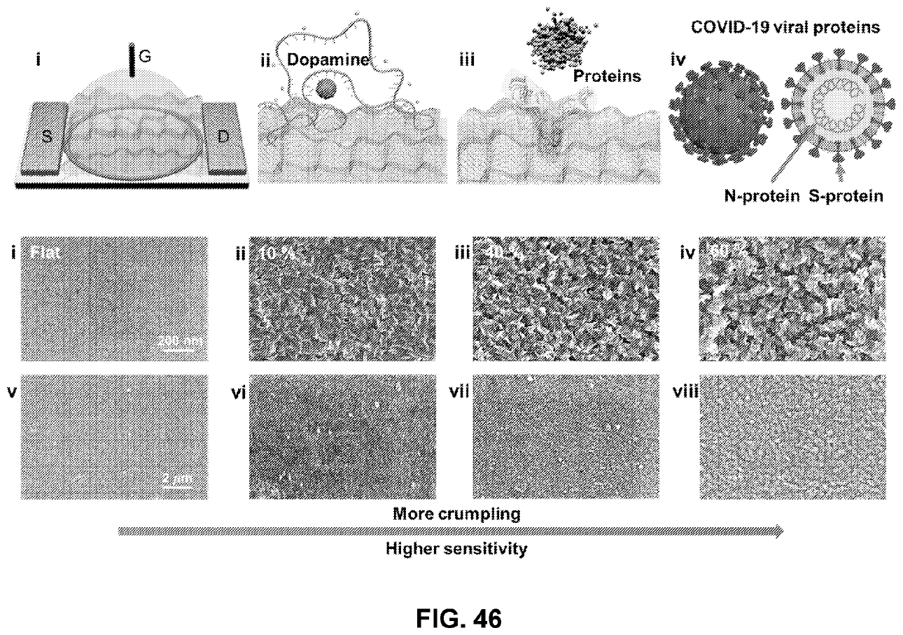

As illustrated in previous examples, a crumpled graphene FET-based biosensor is capable of detecting nucleic acids with ultra-high sensitivity, up to 18 molecules in 50 μL^{[3]}. In this example, we demonstrate that the crumpled graphene can be universally used for different analytes and biomarkers across a range of sizes at an unprecedented concentration level (FIG. 46). Dopamine, Interleukin 6 (IL-6) protein and SARS-CoV-2 Viral spike protein were detected on the crumpled graphene biosensor (FIG. 46). A few biomolecules in 50 μL were clearly detectable and the high sensitivity was possible by optimizing the crumpling ratio of the graphene sensing film. The previous examples show that 18 molecules of DNA can be detectable with 45˜50% of crumpling ratio^{[3]}. As shown in FIG. 46, we also investigate the effect of crumpling ratio (10%˜60%) on biosensing performance, as further explained below.

Importantly, we successful detected COVID-19 related proteins, spike (S-) and nucleocapsid (n-) proteins with extremely low LODs.

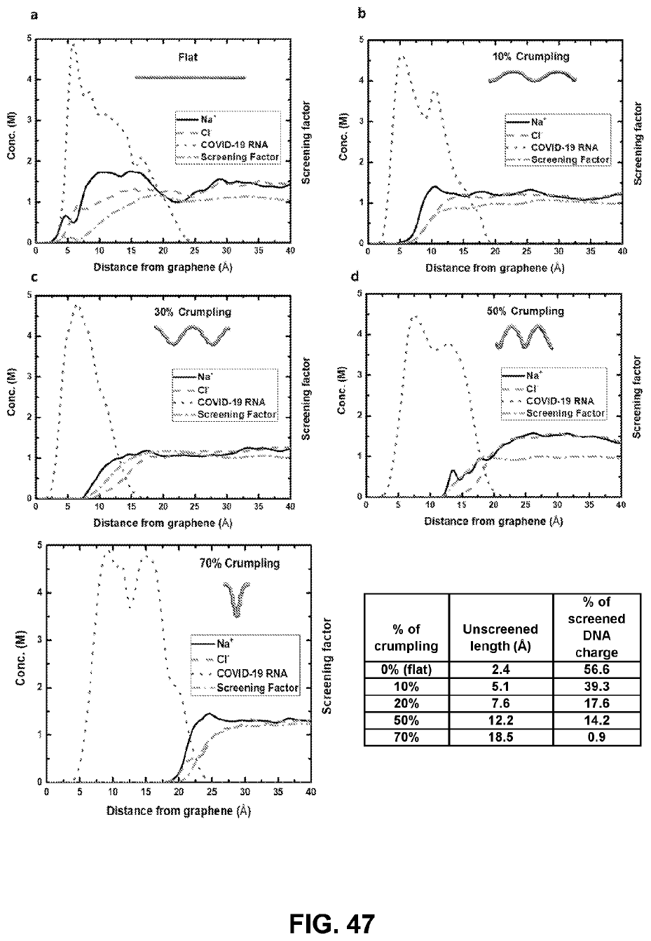

Molecular dynamics (MD) Simulations: We investigate the effect of RNA and ionic species near flat and crumpled graphene surfaces to predict and explain the enhanced sensitivity of molecular detection from our experimental observations. As shown in FIG. 47, five different configurations are considered (see the computational methods for the simulations details). In the first configuration, which is labeled as flat, a COVID-19 RNA with 20 bases is equilibrated on a flat graphene sheet. In the other four systems, the RNA is self-adsorbed onto the surface of the crumpled graphene along the concave regions of the graphene for different degrees of crumpling of 10%, 30%, 50% and 70%. It has been shown that DNA molecules are adsorbed onto the concave region of crumpled graphene, as it is the most energetically favorable configuration^{[3]}. The detection sensitivity of any charge molecule is influenced by the degree by which the charged molecule is screened by the EDL. Detection of the RNA molecule is enhanced when more of the RNA charge is exposed to the graphene surface without being electrostatically screened by ions. The concentrations of ions (sodium and chloride) and the backbone of the RNA strand as well as the screening factor of ions are plotted in FIG. 47 for all the configurations considered. The screening factor is obtained from:

$$S F ( z ) = {\int }_{0}^{z} F ( [ {Na}^{+} ] - [ {Cl}^{-} ] ) dz / \vert σ \vert ,$$

where F is the Faraday constant, z is the normal distance from graphene surface (z=0 on graphene) and σ is the surface charge density. As shown, because of the confined nature of the concave crumpled region, the ionic layer forms farther away from the concave graphene surface compared to the case of flat graphene. As the degree of crumpling increases, more ions are excluded from the graphene interface and the ionic screening takes place at a longer distance away from the graphene. With less of the RNA charge screened in highly crumpled graphene sheets, the RNA detection is enhanced. Hence, it should be noted that performance of crumpled graphene can be correlated with the crumpling ratio and systemically optimized for improved performance. The crumpling ratio was controlled by varying the annealing temperature (110˜115° C.) and time (10˜120 minutes).

To investigate the electronic structure of the deformed graphene, we perform density functional theory (DFT) calculations using the local density approximation (LDA)^{[34]} and Perdew-Burke-Ernzerh (PBE)^{[35]} functionals. Both LDA and PBE functionals produce bandgap values that are close to each other and show a local bandgap opening in the deformed graphene over all the crumpling range (10-60%) studied here. While the bandgap exhibits an oscillatory behavior as a function of the crumpling percentage, the overall bandgap opening upon crumpling has been shown to play an important role in increasing the graphene detection sensitivity^{[3]}.

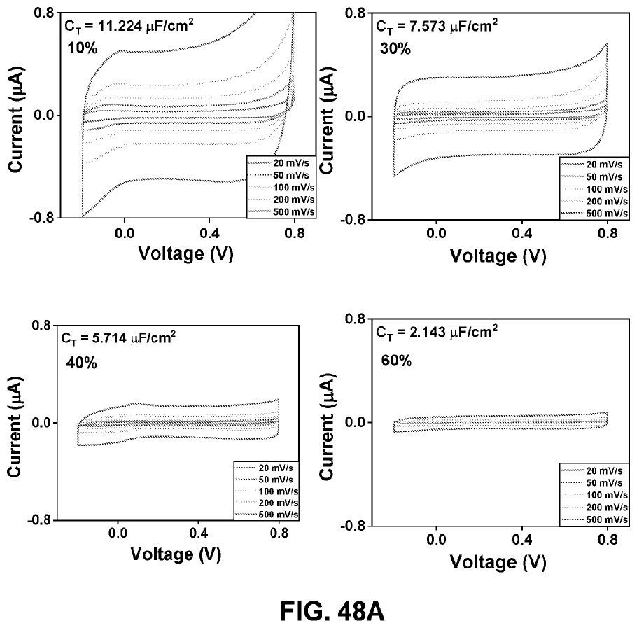

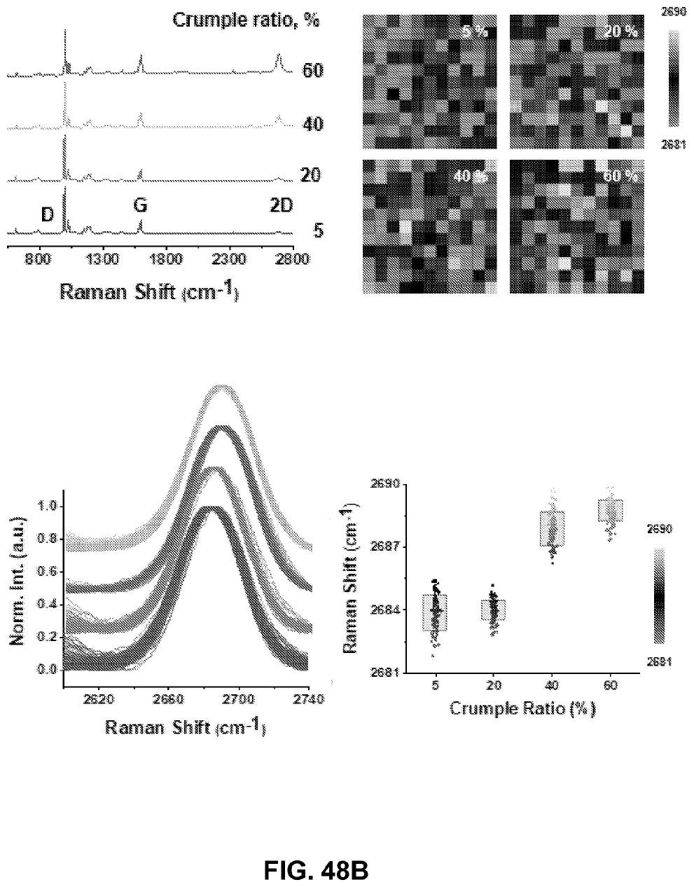

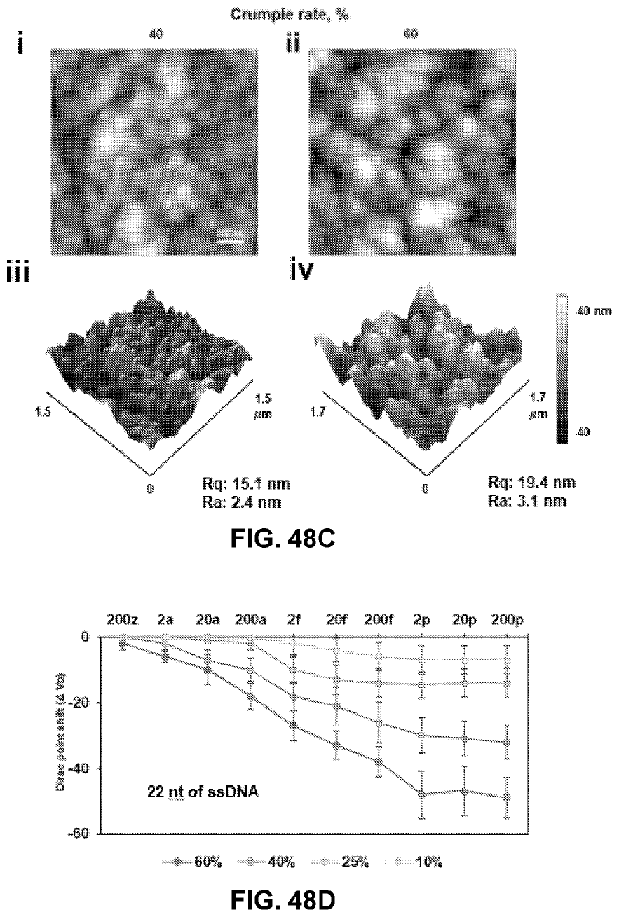

Surface Characterization: Surface characterization of the crumpled graphene with different ratios was performed with AFM and SEM imaging, contact angle measurements, EDL capacitance measurements and Raman spectroscopy analysis (FIG. 48A-48D). There are studies which report structural analysis, roughness changes and hydrophobicity modulation with stepwise crumpling of graphene by stretching and releasing on elastic substrates[^{19],[20],[21]}. However, there are few reports on graphene crumpling with slow buckling on a thermoplastic with a stepwise manner^{[22],[23],[24],[25]}. Surface analysis of the different ratios of graphene crumpling is explored by AFM imaging. As shown in FIG. 48C, increasing the crumpling ratio creates more complicated ripples of graphene. The roughness values increase with higher crumpling ratios. More complicated and rougher surface should perturb ions distribution at the interface of the graphene and ionic solutions more effectively. Also, higher ratio of crumpling increases the surface area of graphene per area; thus the surface becomes more hydrophobic^{[26]}. Those two effects result in modulation of Debye screening effect.

Hydrophobicity heavily influences the formation of the EDL and it is directly related to Debye screening effect thus affect the sensitivity of the charge-based sensors^{[27]}. The hydrophobicity of crumpled graphene samples with four different crumpling ratios is compared by contact angle measurements. It has already been shown that the hydrophobicity of MoS₂ sheets on the pre-strained polystyrene substrate can be tuned by different crumpling ratio with the similar experimental schematics with this work^{[26]}. In the previous study on MoS2, higher crumpling ratio provided higher hydrophobicity. Similar to this result, 60% crumpled graphene showed the highest hydrophobicity with the largest contact angle while 15% crumpling showed the smallest contact angle. EDL capacitance measurements results were also consistent with the contact angle data (FIG. 48A). 55% crumpling showed the smallest capacitance, which indicated the highest hydrophobicity. Therefore, it would be a rational conclusion that 60% of crumpling should provide the highest sensitivity. However, it tends that wettability of the graphene decreases as the crumpling ratio increases. We have found that it might be hard to settle on Wenzel state at certain highly crumpling ratio, resulting in unstable Dirac points measurement over time.

Because of increased hydrophobicity, crumpled graphene devices need ‘wetting process’ by repeating the I-V measurements over hours without adding target molecules. When the Dirac points are identical for at least two hours, the actual biomarker detection experiments can be initiated. We found that devices with higher crumpling required a longer time for the wetting process. Most of devices with 50% crumpled ratios required up to 4 hours of wetting process, while flat devices did not need any prior incubation time for wetting. When the crumpling ratio was 60%, some devices needed more than one day of wetting and even after the Dirac point being stable, 25% of the devices yielded reliable and consistent sensing results. These issues could be attributed to irreversible bonding of some ions with graphene with prolonged exposure to water^{[28]}. Hence, increase in sensitivity of devices is also correlated with decreasing yield and we concluded that 50%-55% is an optimal crumpling ratio when considering the trade-offs between sensitivity, reproducibility and yield.

It is known that strain effect can open the bandgap of graphene. We have shown that the strain induced by crumpling process may locally open the bandgap of the graphene. The strain can be quantified by analyzing Raman G and 2D mode^{[23],[29]}. It has been reported that the strain modulation of graphene by nanoscale substrate curvatures can be characterized by measuring shifts of G and 2D peaks^{[30]}. Also, the rippled graphene can behave as a semiconductor due to the strain^{[31]}. Moreover, larger strain may open larger bandgap^{[32]}. Therefore, if increasing the crumpling ratio generates larger strain, the bandgap opening effect can be larger with higher crumpling ratio and this would contribute to larger exponential changes in the current from a small numbers of charges^{[33]}.

Typical raw spectra Raman peaks of crumpled graphene with different crumpling ratios are shown in FIG. 48B. The size of the laser spot in the Raman measurements is ˜0.2×0.2 μm², covering many peaks and valleys of various sizes of crumples. Thus, the spatially averaged strain across crumples was measured in each spectrum spot. It is reasonable to consider those values as estimates of the overall magnitude of strain even though these averaged measurements is not the complete information of the microscopic strain distribution. It is well-known that two prominent peaks are closely related to strain effect on graphene: G mode (1580-1590 cm⁻¹) and 2D mode (2660-2680 cm⁻¹). However, G peaks of the polystyrene substrate and graphene overlap, making only the 2D mode peaks useful^{[3]}. Compared to 5% crumpled graphene, 20, 40 and 60% show blue shifts in the 2D peaks, with more significant shifts occurring in larger crumpling ratios. In FIG. 48B, the measured spectrum in each optical pixel is fitted with Lorentzian line shapes for 2D modes, and the fitted peak values (ω_{2D}) are plotted in the map. The strong modulation on the Raman scattering of crumpled graphene was confirmed by the sharp transition across the domain boundary and the large spectral shifts. Raman peak shifts in graphene are known to be sensitive to strain modulations^{[23], [29],[30]} The strain is tensile if the shift is blue and compressive when it is red. The average strain effect of different crumpling ratios shows a clear increasing trend. All the larger crumpling ratios samples have larger average tensile strain than the smallest crumpling ratio, 5%, revealing that the graphene lattice is stretched with larger crumpling ratios. It was reported that the stretched graphene showed bandgap opening and the gap can be larger when the tensile strain was increased^{[32]}. It is reasonable to conclude that the larger crumpling ratio might open larger bandgap of graphene because of the larger strain and this can lead to the higher sensitivity in charge-based biomolecular sensing^{[3],[33]}.

To clarify the physical distinction of highly crumpled graphene, 40% and 60% of crumpled graphene samples were imaged by atomic force microscopy (AFM) (FIG. 48C). Two-dimensional images of AFM images were not so distinguishable at glance. However, three-dimensional images show clearly higher fluctuation in z-direction for the 60% sample as compared to the 40%. Rq and Rs values are also higher as marked in FIG. 48C.

DNA absorption through 7E-7E stacking on the graphene FET was also investigated with varied crumpling ratios and electrical measurements (FIG. 48D). Four different crumpling ratios ranging from 10% to 55% has been tested in 1× PBS buffer solution. As predicted, the 55% showed the largest Dirac shift of 50 mV while the 30% showed 15 mV. LODs were also different; there was 8 mV of shift with the 55% crumpling at 200 zM, which corresponds to only —6 molecules. However, similar size of shift was observed at 20 aM with 40% of crumpling and negligible shifts were observed below this concentration. Noticeable shifts were observed from 2 fM for 30% of crumpling. In the previous study, flat device does not generate meaningful signals with the similar experimental condition. From the above results, we optimized the crumpling ratio to 55% and used this ratio for the sensing experiments performed next. We note that the rest of the device fabrication, sample preparation including the probe molecule immobilization, and electrical measurement methods are as described above^{[3]}. The sensitivity enhancement effect induced by the crumpling process can be applied to charge-based molecular detection.

Detection of Small Molecule Dopamine: We first examine the detection of an uncharged small molecule, dopamine, which is a neurotransmitter associated with motivational salience, can serve as a biomarker for the onset and progression of diverse diseases such as schizophrenia, Parkinson's disease, and several cancers such as neuroblastoma and pheochromocytoma^{[4]}. Conventional monitoring methods of upregulation, downregulation or imbalance of dopamine are challenged by rigorous sample preparation to achieve the desired specificity and sensitivity. Precise detection of dopamine can be closely related to early diagnosis of neurological diseases, function tests of dopaminergic neurons derived from various stem cell sources and toxicity assessments of drugs^{[36]}. FET-based dopamine detection can solve those limitations. Dopamine is electrically neutral however, a previous study showed that the neutral molecules can be detectable with conformational change of aptamer probes^{[9]}. Graphene- and its nanocomposite-based electrical or electrochemical sensing of dopamine have been demonstrated previously however, as dopamine is electroneutral, the detections relied on redox or oxidation process of analytes, which lack specificity and sensitivity^{[5],[6],[7]}. A few studies utilized aptamer as a probe molecule for aptamer detection and one report demonstrated Indium oxide FET-based dopamine detection at fM range of concentration with conformation change of aptamer probe when capturing the target molecules, which does not need complicated sample preparation process^{[8],[9]}. Precise detection of dopamine can be closely related to early diagnosis of neurological diseases, function tests of dopaminergic neurons derived from various stem cell sources and toxicity assessments of drugs^{[36]}.

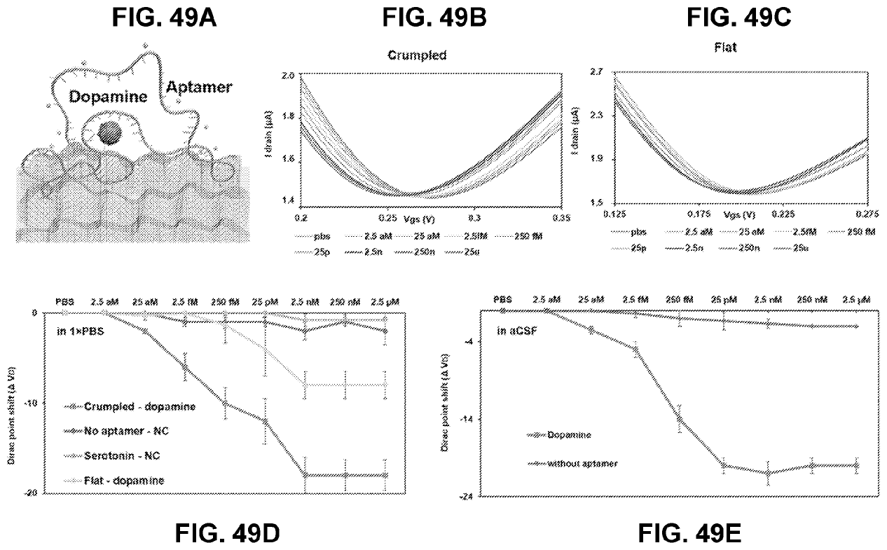

An aptamer is a single stranded DNA or RNA, when its conformation is changed, the distance of the overall charge from the negative backbone to the active channel surface can be changed and its electrical properties can be modulated. In this previous report where the same aptamer was used on an organic FET biosensor, the aptamer, which is negatively charged, became closer to the active channel surface when capturing dopamine^{[9]}. This situation can be analogous to DNA capturing with probe molecules and it has been reported that the IV curve of the proposed graphene FET biosensor shifts to left when DNA capturing happens^{[3]}. As seen in FIG. 49A-49C, capturing of dopamine by the aptamer induced left-shift of the IV curve which is consistent with the previous reports. The dopamine aptamer-FET exhibited concentration-dependent responses to dopamine, ranging from 2.5 aM to 2.5 μM). The LoD was 25 aM (FIG. 49D), which is 3 orders of magnitude lower than the previous report with the same aptamer. Negative control tests were performed with non-specific target molecule, serotonin as well as with dopamine without the aptamer probe. Negative control signals were negligible compared to the positive test signals. Dopamine detection was also reproduced on a regular flat graphene FET and the sensitivity was far inferior (FIG. 49E).

Detection of IL-6 Protein: Larger size molecules, proteins such as interleukin-6 (IL-6) and viral protein were also detected on the crumpled graphene FET biosensor with the highest sensitivity ever reported herein^{[1],[10],[11]}. IL-6 protein is a multipotent cytokine that plays an important role in immune responses, inflammation, bone metabolism, reproduction, arthritis, aging and neoplasia^{[12]}. Monitoring the level of IL-6 protein can help diagnose many inflammatory diseases and cancers including sepsis. Concentration level of IL-6 in blood can grow up to ˜nM range^{[13]}. However, several reports proposed advanced diagnostics in non-invasive manners from saliva, sweat or urine, and those solutions contain much lower concentration of IL-6, thus require highly sensitive sensing platform^{[14],[15]}.

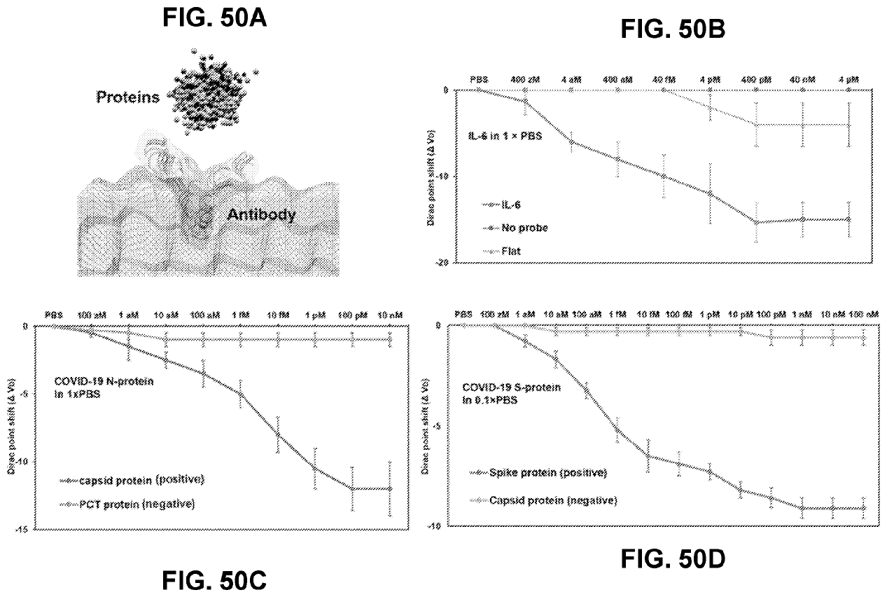

We examine the detection of Interleukin 6 (IL-6) as shown in FIG. 50A and 50B. To bind the inherently charged IL-6 protein, IL-6 antibody was used as a probe molecule. It was reported that the detection of IL-6 protein with the antibody on a graphene FET showed negative shift of the IV curves^{[10]}. We also consistently found negative shifts of Dirac points with the detection of IL-6 protein with the antibody on the crumpled graphene FET in 1× PBS (FIG. 50B). The total shift is smaller than previously reported DNA detection because there can be certain distances from the graphene surface to the charged target protein (˜25 kD) due to the bulkiness of the antibody as the general IgG molecular weight is ˜150 kDa. The isoelectric point of IL-6 protein is pH 6.96 therefore it can be assumed that the overall charge of the protein is negative in PBS solution which could explain the left-shifts of the IV curves. The flat device was not able to detect IL-6 protein as the protein is further away and screened as Debye length of 1× PBS is ˜1 nm. In summary, we demonstrate detection of IL-6 protein detection by using its antibody as a probe molecule on the crumpled graphene FET in 1× PBS and showed aM level sensitivity, which corresponds to only tens of molecules in 50 μL, given sample volume.

Detection of SARS CoV-2 Antigens: There are three kinds of commercially available tests for COVID-19. These are; (i) detection of viral RNA genes based on nucleic acid amplification techniques are the gold standard for confirmation of COVID-19, (ii) Antigen tests which target the proteins on or inside the virus. The antigen tests do not need amplification and hence can significantly reduce the turnaround time but are inherently less sensitive as compared to the RNA tests, and (iii) antibody tests from blood samples which detect presence of antibodies and confirm immunity from the infection. There is an opportunity to improve the sensitivity of the antigen tests and use of label free electric sensors could play a role in this area. For SARS-CoV-2, the spike protein or the nucleocapsid protein can be captured with specific antibodies anchored on surfaces. Several antigen tests are available or in development by companies including Quidel, OraSure, Iceni Diagnostics, and E25Bio. These antigen tests are typically based on enzyme-linked immunoassay (ELISA) and have an optical readout. New tests typically require fluorescence labelling or nanoparticle anchoring to enhance the output signal. There are reports which have raised concerns about the performance of available antigen tests^{[16],[17]}. For example, an analysis suggest that each 10-fold increase in LoD is expected to increase the false negative rate by 13%, missing an additional one in eight infected patients^{[17]}.

The crumpled graphene FET-based antigen test can be important as its sensitivity can be superior to ELISA and it does not require any labelling^{[18]}. Such sensors can attempt to capture whole virus or the proteins after the virus lysis, the later can be safer from a user perspective. Recent reports have demonstrated label-free COVID-19 related protein detection in human nasopharyngeal swab specimens on graphene FET sensors^{[1]}.

We immobilized N- and S-protein antibodies on the graphene channels as reported previously^{[1]}. Different kinds of coronavirus-related N-proteins detections were reported using nanowire FET in the pM concentrations^{[37],[38]}, however an improvement in sensitivity desired if antgen tests were to replace the RNA molecular detection tests^{[17]}. Moreover, COVID-19 N-protein has not been tested on any kinds of FET based biosensor before this report. FIG. 50C shows results of the IV-curve shifts with N-protein detection in 1× PBS on the crumpled graphene channels. It is noted that many COVID-19 related protein detections were performed in viral transport media (VTM), not only PBS. This is because VTM, which preserves the virus for some period, is the current standard transport solution for collection of COVID-19 clinical samples for molecular tests such as PCR. However, antigen tests do not necessarily need VTM as a media as the tests are designed to be performed soon after collecting the samples^{[16]}. In fact, the virus should be lysed to open and free specific viral proteins for antigen tests. PBS is used in antigen tests in this context^{[39]}.

The isoelectric point of the N-protein is pH˜10 and thus the overall charge would be positive in 1× PBS. However, previous studies showed that some parts of the SARS N-protein, of which the isoelectric point is also ˜10, are negatively charged. It might be possible that the positive charges on active channel surface attract the negatively charged regions of N-protein captured on the surface, thus leading to conductance change as a result of the local negative charges of the antigens at the surface^{[37],[38]}. This result was supported by another literature with computational simulations, which shows that N-protein has an asymmetric charge distribution. Depends on its ‘up’ or ‘down’ orientation, the protein would generate different charge signals at pH=7.4. It was concluded that, with ‘up orientation’, the N-protein is expected as negative charges for Debye screening lengths up to 3 nm^{[40]}. Even though these previous results were based on SARS N-protein and not the SARS-CoV-2 N-protein, their isoelectric points and molecular weights are very similar, i.e. pH˜10 and 46 kD, respectively. Furthermore, it was recently confirmed that many aspects of SARS N-protein and COVID-19 N-protein are similar and they show more than 90% sequence similarity^{[41]}. Thus it is reasonable that electrical charge effect of COVID-19 n-protein on graphene surface could be either positive or negative depends on the orientation^{[}42],[43].

The S-protein was also tested on the crumpled graphene FET (FIG. 50D) and showed a clear shift at 1 aM in 0.1× PBS. In 1× PBS, the signal from S-protein detection was not statistically significant and could not be delineated. This can also be explained by large size of the S-protein antibody at ˜150 kD and the decrease in Debye Length from 0.1× to 1× PBS. Many prior studies have reported on the differences in signal with size of antibodies as function of experimental conditions, including buffer solution concentrations, etc.^{[1],[37],[38],[44]}.

In summary, detection of several important disease-related biomarkers including dopamine, IL-6 protein and viral proteins were demonstrated on the crumpled graphene FET biosensor. The effect of various crumpling ratio on the detection sensitivity was studied guided and confirmed by computational simulations, Raman spectroscopy analysis, and EDL capacitance measurements. The optimized crumpled graphene FET biosensors were able to detect various size of biomarkers with unprecedented sensitivities. Importantly, we also demonstrated SARS CoV-2 antigen test by detecting S- and N-proteins with the crumpled graphene FET biosensor in a label-free format that could result in a diagnostic tool with small footprint. This platform can overcome the limitation of currently available antigen tests owing to its superior sensitivity due to modulation of the Debye layer on the crumpled graphene biosensors. In the future, this platform can be highly multiplexed and can target many multiple biomarkers on a chip and have a significant impact on the diagnostics market.

Methods

MD simulations: Molecular dynamics simulations were performed using the LAMMPS package³. The systems were generated by the Visual Molecular Dynamics (VMD)⁴. As mentioned in the manuscript, different graphene topologies with crumpling degrees of 0% (flat), 10%, 30%, 50% and 70% are used. Each simulation box consists of a single-layer graphene sheet, a single-stranded COVID-19 RNA, water and ions. The COVID-19 RNA segment has 20 bases with a sequence of GAC CCC AAA ATC AGC GAA AT (SEQ ID NO:18). A concentration of 1.2M Sodium chloride is considered. Depending on the degrees of crumpling, the simulation systems contain 20,000 to 80,000 atoms. Periodic boundary conditions are applied in the x and y directions (projected plane of graphene lies in the xy plane). The systems are non-periodic in z direction. The CHARMM forcefield⁵ is used. The Lennard-Jones (LJ) potential with a cutoff distance of 1.2 nm is employed. The long—range electrostatic interactions are calculated using the PPPM⁶.

First, the energy of each system was minimized for 15,000 steps. Equilibrium simulations were then performed in NPT ensemble for 2 ns at a pressure of 1 atm and a temperature of 300 K. The NPT simulation ensures that the water reaches its equilibrium density. The systems were further equilibrated in NVT ensemble for another 2ns at 300 K. Temperature was maintained at 300 K by using the Nose-Hoover thermostat^{7,8} with a time constant of 0.1 ps. The carbon atoms of graphene are kept frozen. The production simulations were carried out in NVT ensemble for 10 ns. Trajectories of atoms were dumped every picosecond to obtain the structure of DNA and ions near graphene sheets.

Atomic Force Microscopy imaging: AFM images were recorded using an ASYLUM RESEARCH MFP-3D AFM SYSTEM (Asylum Research, Santa Barbara, Calif.).

Raman Spectroscopy imaging: The Raman spectra and imaging of the crumpled graphene were recorded using a Nanophoton RAMAN-11 laser confocal microscope (Nanophoton, Osaka, Japan). A 532 nm diode laser was used for excitation.The excitation power was 0.1 mW with 3 s exposure time and 3 times averaging for point and mapping, respectively. In Raman mapping mode, region of interest was 2.2 by 2.2 um in x, y axis with 200 nm/pixel resolution with NA 0.9 100× Plan Fluor objective lens. The grating was 600 gr/mm. The wave number range covered was 700-2900 cm−1. The wavenumber shift compensation was −8.4 cm−1 after initial Ne-sample calibration. The Raman signals were detected by a Peltier cooled CCD camera at −70° C.

Example 3 References:
[1] G. Seo, G. Lee, M. J. Kim, S.-H. Baek, M. Choi, K. B. Ku, C.-S. Lee, S. Jun, D. Park, H. G. Kim, S.-J. Kim, J.-O. Lee, B. T. Kim, E. C. Park, S. Il Kim, ACS Nano 2020, 14, 5135.
[2] T. Ji, Z. Liu, G. Wang, X. Guo, S. Akbar khan, C. Lai, H. Chen, S. Huang, S. Xia, B. Chen, H. Jia, Y. Chen, Q. Zhou, Biosens. Bioelectron. 2020, 166, 112455.
[3] M. T. Hwang, M. Heiranian, Y. Kim, S. You, J. Leem, A. Taqieddin, V. Faramarzi, Y. Jing, I. Park, A. M. van der Zande, S. Nam, N. R. Alum, R. Bashir, Nat. Commun. 2020, 11, 1543.
[4] A. Vazquez-Guardado, S. Barkam, M. Peppler, A. Biswas, W. Dennis, S. Das, S. Seal, D. Chanda, Nano Lett. 2019, 19, 449.
[5] A. Pandikumar, G. T. Soon How, T. P. See, F. S. Omar, S. Jayabal, K. Z. Kamali, N. Yusoff, A. Jamil, R. Ramaraj, S. A. John, H. N. Lim, N. M. Huang, RSC Adv. 2014, 4, 63296.
[6] J. Oh, J. S. Lee, J. Jun, S. G. Kim, J. Jang, ACS Appl. Mater. Interfaces 2017, 9, 39526.
[7] M. Zhang, C. Liao, Y. Yao, Z. Liu, F. Gong, F. Yan, Adv. Funct. Mater. 2014, 24, 978.
[8] B.-R. Li, Y.-J. Hsieh, Y.-X. Chen, Y.-T. Chung, C.-Y. Pan, Y.-T. Chen, J. Am. Chem. Soc. 2013, 135, 16034.
[9] N. Nakatsuka, K.-A. Yang, J. M. Abendroth, K. M. Cheung, X. Xu, H. Yang, C. Zhao, B. Zhu, Y. S. Rim, Y. Yang, P. S. Weiss, M. N. Stojanovié, A. M. Andrews, Science (80-.). 2018, 362, 319 LP.
[10] J. Huang, J. Harvey, W. H. D. Fam, M. A. Nimmo, I. Y. A. Tok, Procedia Eng. 2013, 60, 195.
[11] J. Huang, H. Chen, W. Niu, D. W. H. Fam, A. Palaniappan, M. Larisika, S. H. Faulkner, C. Nowak, M. A. Nimmo, B. Liedberg, A. I. Y. Tok, RSC Adv. 2015, 5, 39245.
[12] X.-P. JIANG, D. C. YANG, R. L. ELLIOTT, J. F. HEAD, Anticancer Res. 2011, 31, 2899.
[13] J. Angulo, C. Martinez-Valdebenito, C. Marco, H. Galeno, E. Villagra, L. Vera, N. Lagos, N. Becerra, J. Mora, A. Bermudez, J. Diaz, M. Ferrés, M. López-Lastra, PLoS Negl. Trop. Dis. 2017, 11, e0005757.
[14] S. Kumar, J. G. Sharma, S. Maji, B. D. Malhotra, Biosens. Bioelectron. 2016, 78, 497.
[15] M. D. Hladek, S. L. Szanton, Y.-E. Cho, C. Lai, C. Sacko, L. Roberts, J. Gill, J. Immunol. Methods 2018, 454,1.
[16] A. Scohy, A. Anantharajah, M. Bodéus, B. Kabamba-Mukadi, A. Verroken, H. Rodriguez-Villalobos, J. Chn. Virol. 2020, 129, 104455.
[17] R. Arnaout, R. A. Lee, G. R. Lee, C. Callahan, C. F. Yen, K. P. Smith, R. Arora, J. E. Kirby, bioRxiv 2020, 2020.06.02.131144.
[18] C. P. Quinn, V. A. Semenova, C. M. Elie, S. Romero-Steiner, C. Greene, H. Li, K. Stamey, E. Steward-Clark, D. S. Schmidt, E. Mothershed, J. Pruckler, S. Schwartz, R. F. Benson, L. O. Helsel, P. F. Holder, S. E. Johnson, M. Kellum, T. Messmer, W. L. Thacker, L. Besser, B. D. Plikaytis, T. H. Taylor, A. E. Freeman, K. J. Wallace, P. Dull, J. Sejvar, E. Bruce, R. Moreno, A. Schuchat, J. R. Lingappa, S. K. Martin, J. Walls, M. Bronsdon, G. M. Carlone, M. Bajani-Ari, D. A. Ashford, D. S. Stephens, B. A. Perkins, Emerg. Infect. Dis. J. 2002, 8, 1103.
[19] J. Zang, S. Ryu, N. Pugno, Q. Wang, Q. Tu, M. J. Buehler, X. Zhao, Nat. Mater. 2013, 12, 321.
[20] S. Deng, V. Berry, Mater. Today 2016, 19, 197.
[21] D. Rhee, J. T. Paci, S. Deng, W.-K. Lee, G. C. Schatz, T. W. Odom, ACS Nano 2020, 14, 166.
[22] P. Snapp, M. Heiranian, M. T. Hwang, R. Bashir, N. R. Alum, S. Nam, Curr. Opin. Solid State Mater. Sci. 2020, 24, 100836.
[23] W.-K. Lee, J. Kang, K.-S. Chen, C. J. Engel, W.-B. Jung, D. Rhee, M. C. Hersam, T. W. Odom, Nano Lett. 2016, 16, 7121.
[24] P.-Y. Chen, J. Sodhi, Y. Qiu, T. M. Valentin, R. S. Steinberg, Z. Wang, R. H. Hurt, I. Y. Wong, Adv. Mater. 2016, 28, 3564.
[25] W. Chen, X. Gui, L. Yang, H. Zhu, Z. Tang, Nanoscale Horizons 2019, 4, 291.
[26] J. Choi, J. Mun, M. C. Wang, A. Ashraf, S.-W. Kang, S. Nam, Nano Lett. 2017, 17, 1756.
[27] S. S. Kwon, J. Choi, M. Heiranian, Y. Kim, W. J. Chang, P. M. Knapp, M. C. Wang, J. M. Kim, N. R. Alum, W. Il Park, S. Nam, Nano Lett. 2019, 19, 4588.
[28] S. S. Kwon, J. Yi, W. W. Lee, J. H. Shin, S. H. Kim, S. H. Cho, S. Nam, W. Il Park, ACS Appl. Mater. Interfaces 2016, 8, 834.
[29] J. E. Lee, G. Ahn, J. Shim, Y. S. Lee, S. Ryu, Nat. Commun. 2012, 3, 1024.
[30] Y. Zhang, M. Heiranian, B. Janicek, Z. Budrikis, S. Zapperi, P. Y. Huang, H. T. Johnson, N. R. Alum, J. W. Lyding, N. Mason, Nano Lett. 2018, 18, 2098.
[31] U. Monteverde, J. Pal, M. A. Migliorato, M. Missous, U. Bangert, R. Zan, R. Kashtiban, D. Powell, Carbon N. Y. 2015, 91, 266.
[32] Z. H. Ni, T. Yu, Y. H. Lu, Y. Y. Wang, Y. P. Feng, Z. X. Shen, ACS Nano 2008, 2, 2301.
[32] D. Sarkar, W. Liu, X. Xie, A. C. Anselmo, S. Mitragotri, K. Banerjee, ACS Nano 2014, 8, 3992.
[34] Ceperley, D. M.; Alder, B. J., Ground State of the Electron Gas by a Stochastic Method. Physical Review Letters 1980, 45, 566.
[35] Perdew, J. P.; Burke, K.; Ernzerhof, M., Generalized Gradient Approximation Made Simple. Phys Rev Lett 1996, 77, 3865.
[36] D.-S. Kim, E.-S. Kang, S. Baek, S.-S. Choo, Y.-H. Chung, D. Lee, J. Min, T.-H. Kim, Sci. Rep. 2018, 8, 14049.
[37] F. N. Ishikawa, H.-K. Chang, M. Curreli, H.-I. Liao, C. A. Olson, P.-C. Chen, R. Zhang, R. W. Roberts, R. Sun, R. J. Cote, M. E. Thompson, C. Zhou, ACS Nano 2009, 3, 1219.
[38] F. N. Ishikawa, M. Curreli, C. A. Olson, H.-I. Liao, R. Sun, R. W. Roberts, R. J. Cote, M. E. Thompson, C. Zhou, ACS Nano 2010, 4, 6914.
[39] G. C. K. Mak, P K. C. Cheng, S. S. Y. Lau, K. K. Y. Wong, C. S. Lau, E. T. K. Lam, R. C. W. Chan, D. N. C. Tsang, J. Clin. Viral. 2020, 129, 104500.
[40] M. R. Hediger, K. L. Martinez, J. Nygård, M. Brandbyge, J. H. Jensen, L. De Vico, in (Eds.: S. Li, J. Wu, Z.M. Wang, Y. Jiang), Springer International Publishing, Cham, 2013, pp. 55-86.
[41] B. Tilocca, A. Soggiu, M. Sanguinetti, V. Musella, D. Britti, L. Bonizzi, A. Urbani, P. Roncada, Microbes Infect. 2020, 22,188.
[42] R. J. Chen, H. C. Choi, S. Bangsaruntip, E. Yenilmez, X. Tang, Q. Wang, Y.-L. Chang, H. Dai, J. Am. Chem. Soc. 2004, 126, 1563.
[43] C.-T. Lin, P. T. K. Loan, T.-Y. Chen, K.-K. Liu, C.-H. Chen, K.-H. Wei, L.-J. Li, Adv. Funct. Mater. 2013, 23, 2301.
[44] X. Zhang, Q. Qi, Q. Jing, S. Ao, Z. Zhang, M. Ding, M. Wu, K. Liu, W. Wang, Y. Ling, arXiv Prepr. arXiv2003.12529 2020.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every device, system, formulation, combination of components, or method described or exemplified herein can be used to practice the invention, unless otherwise stated.

Whenever a range is given in the specification, for example, a size range, a volume range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A biosensor comprising:
a field effect transistor (FET) comprising:
a source electrode;
a drain electrode, wherein the source and drain electrodes are separated from each other by an electrode separation distance;
a channel layer between the source electrode and the drain electrode, wherein the channel layer has a crumpled geometry;
a sample reservoir in fluidic contact with the channel layer, wherein the sample reservoir is configured to hold a sample solution;
a gate electrode configured to electrically contact the sample solution in the sample reservoir; and
wherein the crumpled geometry of the channel increases a detection limit of the biosensor to molecules in an ionic solution.

2. The biosensor of claim 1, wherein the channel layer is formed of a two-dimensional layer of material selected from the group consisting of: graphene, doped silicon, silicene, ultra-thin metal, germanane, MoS₂, and dichalcogenides.

3. The biosensor of claim 1, wherein the channel layer is formed of graphene.

4. The biosensor of claim 1, further comprising a support substrate layer that supports the electrodes and channel layer, wherein the support substrate is formed of a material capable of undergoing a shrinkage transformation to thereby crumple the channel layer that is supported by the support substrate.

5. The biosensor of claim 1, further comprising a probe anchored to the channel by a linker molecule, wherein the probe is selected from the group consisting of: a polynucleotide, a peptide nucleic acid (PNA) probe, an aptamer, a protein, an antibody, and a capture agent, wherein the probe has a sequence selected to specifically bind a target molecule.

6. (canceled)

7. (canceled)

8. The biosensor of claim 1, wherein during use with the sample solution, a Debye length at the surface of the crumpled geometry is greater than a Debye length of an equivalent channel having a flat geometry.

9. The biosensor of claim 1, wherein the sample comprises a biomolecule selected from the group consisting of a protein, a DNA sequence, an RNA sequence, and any fragments thereof; and the biological solution is unprocessed whole blood, plasma, saliva or sputum.

10. The biosensor of claim 1, wherein the crumpled geometry corresponds to a multi-axial deformation or a uniaxial deformation of the channel layer.

11. The biosensor of claim 1, wherein the crumpled geometry corresponds to an average periodicity ranging from between 1 nm and 100 nm and an average amplitude ranging from between 1 nm and 100 nm.

12. The biosensor of claim 1, wherein the channel layer is in continuous contact or discontinuous contact with a support substrate layer.

13. (canceled)

14. (canceled)

15. A method of detecting a biomolecule, the method comprising the steps of:
providing the biosensor of claim 1;
introducing the sample solution to the channel layer;
applying a gate voltage to the sample solution; and
monitoring a FET electrical parameter, wherein a change in the FET electrical parameter corresponds to presence of the biomolecule in the sample solution.

16. (canceled)

17. (canceled)

18. (canceled)

19. (canceled)

20. A method of detecting amplification of a target polynucleotide, the method comprising the steps of:
providing a field effect transistor (FET) having a crumpled semiconductor material channel that is configured to form a π-π interaction with single stranded DNA;
conducting an amplification reaction in an amplification solution comprising single stranded DNA (ssDNA) primers to obtain an amplified solution;
contacting the FET with the amplified solution; and
electrically detecting with the FET an amount of ssDNA primer in the amplified solution, thereby detecting amplification.

21. The method of claim 21, wherein the semiconductor material is a two-dimensional layer selected from the group consisting of graphene; MoS₂; dichalcogenides and silicene.

22. The method of claim 21, wherein the FET is a crumpled graphene FET (gFET), wherein the ssDNA primer binds to a surface of the crumpled graphene by noncovalent π-π interaction between hexagonal cells of a crumpled graphene and an aromatic ring of the ssDNA and amplified dsDNA does not bind to the crumpled graphene as strongly as ssDNA due to π-π stacking of aromatic rings in the dsDNA.

23. The method of claim 20, wherein the step of conducting the amplification reaction occurs prior to the contacting step.

24. The method of claim 20, wherein the step of conducting the amplification reaction occurs simultaneously with the contacting step.

25. The method of claim 20, further comprising the steps of:
detecting a FET electrical parameter prior to the step of conducting the amplification reaction to obtain a baseline FET electrical parameter value;
detecting the FET electrical parameter after the step of conducting the amplification reaction to obtain a post-amplification FET electrical parameter value;
comparing the baseline and the post-amplification FET electrical parameter values; and
identifying presence of the target polynucleotide for a statistically significant difference between the baseline and the post-amplification FET electrical parameter values.

26. The method of claim 25, wherein the FET electrical parameter is a change in current at a fixed voltage or a Dirac point shift voltage and the statistically significant difference corresponds to an at least 10% difference of the baseline and post-amplification FET electrical parameter.

27. (canceled)

28. The method of claim 20, wherein the electrically detecting step occurs periodically or continuously during the step of conducting the amplification reaction.

29. The method of claim 20, wherein an initial starting concentration of the target polynucleotide is as low as 8×10⁻²¹ M in the amplification solution.

30. The method of claim 20, wherein negative and positive target polynucleotide amplification solutions are distinguished from each other at a target polynucleotide detection limit of between 4×10⁻²¹ and 1×10⁻¹⁸ M.

31. (canceled)

32. The method of claim 20, wherein the ssDNA primers are provided at a concentration so that after 60-90 minutes of amplification, at least 90% of all available ssDNA primers have been incorporated into amplified dsDNA.

33. The method of claim 20 that is without labels and/or without surface-functionalization.

34. The method of claim 20, wherein the amplification reaction is an isothermal amplification such as loop mediated isothermal amplification (LAMP) reaction; or a polymerase chain reaction (PCR).

35. The method of claim 20, wherein the target polynucleotide is present in the amplification reaction so that ssDNA primers are incorporated into amplified double stranded DNA (dsDNA) amplification product and identification of target polynucleotide comprises identifying a change in a FET electrical parameter measured during the electrically detecting step, including a decrease in a Dirac point shift.

36. The method of claim 20, wherein the target polynucleotide is absent from the amplification reaction so that ssDNA primers are not incorporated into an amplified double stranded DNA (dsDNA) amplification product and identification of no target polynucleotide comprises identifying a no change condition in a FET electrical parameter measured during the electrically detecting step, including a not statistically significant change in a FET electrical parameter.

37. (canceled)

38. (canceled)

39. A system for detecting a target polynucleotide in a sample solution comprising:
a FET having:
a source electrode;
a drain electrode, wherein the source and drain electrodes are separated from each other by an electrode separation distance;
a channel layer between the source electrode and the drain electrode, wherein the channel layer comprises a two-dimensional crumpled semiconductor material;
a channel layer receiving surface that forms part of a sample reservoir, wherein the sample reservoir is configured to hold an amplifiable sample solution;
an electrical detector electrically connected to the FET;
the amplifiable sample solution comprising ssDNA primers and amplification reagents to amplify the target polynucleotide, wherein during use the amplifiable sample solution contacts the channel layer receiving surface of the sample reservoir;
wherein ssDNA primers during use bind to the channel layer receiving surface by a noncovalent π-π interaction between the crumpled semiconductor material and an aromatic ring of the ssDNA at a higher affinity than dsDNA, and the electrical detector is configured to detect a level of ssDNA primer by detection of a change in a FET electrical parameter.

40. (canceled)

41. (canceled)

42. (canceled)
